SEQUENCE LISTING (3 sequences)

SEQ ID NO: 1 - protein, 9 aa, Artificial Sequence
  Description of Artificial Sequence Synthetic peptide
IYSTVASSL

SEQ ID NO: 2 - protein, 9 aa, Artificial Sequence
  Description of Artificial Sequence Synthetic peptide
TYQRTRALV

SEQ ID NO: 3 - protein, 16 aa, Artificial Sequence
  Description of Artificial Sequence Synthetic peptide
SYVPSAEQILEFVKQI

(19) United States
(12) Patent Application Publication
(10) Pub. No.: US 2004/0241842 A1
(43) Pub. Date: Dec. 2, 2004

(54) Stimulation of thymus for vaccination development

(75) Inventor: Richard L. Boyd, Hampton (AU)

Correspondence Address: Shann Kerner, Ph.D., HALE AND DORR LLP, 60 State Street, Boston, MA, 02109, US

(73) Assignee: Monash University

(21) Appl. No.: 10/748,450

(22) Filed: Dec. 30, 2003

Related U.S. Application Data
(63) Continuation-in-part of application No. 10/418,747, filed on Apr. 18, 2003.
Application No. 10/418,747 is a continuation-in-part of application No. 09/977,479, filed on Oct. 12, 2001.
Application No. 09/977,479 is a continuation-in-part of application No. 09/965,394, filed on Sep. 26, 2001, now abandoned.
Application No. 09/965,394 is a continuation-in-part of application No. 09/755,965, filed on Jan. 5, 2001, now abandoned.
Application No. 09/965,394 is a continuation-in-part of application No. 09/755,983, filed on Jan. 5, 2001, now abandoned.
Application No. 09/965,394 is a continuation-in-part of application No. 09/755,646, filed on Jan. 5, 2001, now abandoned.
Application No. 09/965,394 is a continuation-in-part of application No. 09/758,910, filed on Jan. 10, 2001, now abandoned.
Application No. 09/755,983 is a continuation-in-part of application No. 09/795,286, filed on Oct. 13, 2000, now abandoned.
Application No. 09/755,983 is a continuation-in-part of application No. 09/795,302, filed on Oct. 13, 2000, now abandoned.
Application No. 09/755,965 is a continuation-in-part of application No. 09/795,286, filed on Oct. 13, 2000, now abandoned.
Application No. 09/755,965 is a continuation-in-part of application No. 09/795,302, filed on Oct. 13, 2000, now abandoned.
Application No. 09/755,646 is a continuation-in-part of application No. 09/795,286, filed on Oct. 13, 2000, now abandoned.
Application No. 09/755,646 is a continuation-in-part of application No. 09/795,302, filed on Oct. 13, 2000, now abandoned.
Application No. 09/758,910 is a continuation-in-part of application No. 09/795,286, filed on Oct. 13, 2000, now abandoned.
Application No. 09/758,910 is a continuation-in-part of application No. 09/795,302, filed on Oct. 13, 2000, now abandoned.
Application No. 09/795,302 is a continuation-in-part of application No. PCT/AU00/00329, filed on Apr. 17, 2000.
Application No. 09/795,286 is a continuation-in-part of application No. PCT/AU00/00329, filed on Apr. 17, 2000.
(60) Provisional application No. 60/527,001, filed on Dec. 5, 2003.

(30) Foreign Application Priority Data
Oct. 15, 2001 (WO) PCT/AU01/01291
Apr. 15, 1999 (AU) PR9778
Oct. 13, 2000 (AU) PR0745

(51) Int. Cl. A61K 39/38; C12N 5/08

(52) U.S. Cl. 435/372; 424/93.7

(57) ABSTRACT

The present disclosure provides methods for enhancing the response of a patient's immune system to vaccination. This is accomplished by reactivating the thymus. Optionally, hematopoietic stem cells, autologous, syngeneic, allogeneic or xenogeneic, are delivered to increase the speed of regeneration of the patient's immune system. In one embodiment the hematopoietic stem cells are CD34⁺. The patient's thymus is reactivated by disruption of sex steroid mediated signaling to the thymus. In one embodiment, this disruption is created by administration of LHRH agonists, LHRH antagonists, anti-LHRH receptor antibodies, anti-LHRH vaccines or combinations thereof.

### CROSS-REFERENCE TO RELATED APPLICATIONS

This application is a continuation-in-part of U.S. Ser. No. 10/399,213, filed Apr. 14, 2003, which is a national phase filing of PCT AU01/01291, filed Oct. 15, 2001, which is a PCT filing of AU provisional application PR0745, filed Oct. 13, 2000. This application is also a continuation-in-part of U.S. Serial No. 60/527001, filed Dec. 5, 2003. This application is also a continuation-in-part of U.S. Ser. No. 10/418,747, filed Apr. 18, 2003, which is a continuation-in-part of U.S. Ser. No. 09/977,479, filed Oct. 12, 2001, which is a continuation-in-part of U.S. Ser. No. 09/965,394 filed Sep. 26, 2001 (abandoned), which is a continuation-in-part of U.S. Ser. No. 09/755,965, filed Jan. 5, 2001 (abandoned), which is a continuation-in-part of U.S. Ser. No. 09/795,286, filed Oct. 13, 2000, which is a continuation-in-part of AU provisional application PR0745, filed Oct. 13, 2000, and of U.S. Ser. No. 09/795,302, filed Oct. 13, 2000 (abandoned), which is a continuation-in-part of PCT AU00/00329, filed Apr. 17, 2000, which is a PCT filing of AU provisional application PP9778 filed Apr. 15, 1999. Each of these applications is hereby incorporated by reference.

### FIELD OF THE INVENTION

The present disclosure is in the field of response to vaccines in a patient. More particularly, the present disclosure is in the field of improving vaccine response through stimulation and reactivation of the thymus.

### BACKGROUND

The Immune System

The major function of the immune system is to distinguish “foreign” (that is derived from any source outside the body) antigens from “self” (that is derived from within the body) and respond accordingly to protect the body against infection. In more practical terms, the immune response has also been described as responding to “danger” signals. These “danger” signals may be any change in the property of a cell or tissue which alerts cells of the immune system that this cell/tissue in question is no longer “normal.” Such alterations may be very important in causing, for example, rejection of tumors. However, this “danger” signal may also be the reason why some autoimmune diseases start, due to either inappropriate cell changes in the “self” cells targeted by the immune system (e.g., the β-islet cells targeted in Diabetes mellitus), or inappropriate cell changes in the immune cells themselves, leading these cells to target normal “self” cells. In normal immune responses, the sequence of events involves dedicated antigen presenting cells (APC) capturing foreign antigen and processing it into small peptide fragments which are then presented in clefts of major histocompatibility complex (MHC) molecules on the APC surface. The MHC molecules can either be of class I expressed on all nucleated cells (recognized by cytotoxic T cells (Tc)) or of class II expressed primarily by cells of the immune system (recognized by helper T cells (Th)). Th cells recognize the MHC II/peptide complexes on APC and respond; factors released by these cells then promote the activation of either of both Tc cells or the antibody producing B cells which are specific for the particular antigen. The importance of Th cells in virtually all immune responses is best illustrated in HIV/AIDS where their absence through destruction by the virus causes severe immune deficiency eventually leading to death. Inappropriate development of Th (and to a lesser extent Tc) can lead to a variety of other diseases such as allergies, cancer and autoimmunity.

The development of such cells may be due to an abnormal thymus in which the structural organization is markedly altered e.g. the medullary epithelial cells which normally effect more mature thymocytes are ectopically expressed in the cortex where immature T cells normally reside. This could mean that the developing immature T cells prematurely receive late stage maturation signals and in doing so become insensitive to the negative selection signals that would normally delete potentially autoreactive cells. Indeed this type of thymic abnormality was found in NZB mice which develop Lupus-like symptoms (Takeoka et al., 1999) and more recently NOD mice which develop type I diabetes (Thomas-Vaslin et al., 1997; Atlan-Gepner et al., 1999). It is not known how these forms of thymic abnormality develop but it could be through the natural aging process or from destructive agents such as viral infections (changes in the thymus have been described in AIDS patients), stress, chemotherapy and radiation therapy (Mackall et al., 1995; Heitger et al., 1997; Mackall and Gress, 1997)

The ability to recognize antigen is encompassed in a plasma membrane receptor in T and B lymphocytes. These receptors are generated randomly by a complex series of rearrangements of many possible genes, such that each individual T or B cell has a unique antigen receptor. This enormous potential diversity means that for any single antigen the body might encounter, multiple lymphocytes will be able to recognize it with varying degrees of binding strength (affinity) and respond to varying degrees. Since the antigen receptor specificity arises by chance, the problem thus arises as to why the body does not “self destruct” through lymphocytes reacting against self antigens. Fortunately there are several mechanisms which prevent the T and B cells from doing so, and collectively they create a situation where the immune system is tolerant to self.

The most efficient form of self tolerance is to physically remove (kill) any potentially reactive lymphocytes at the sites where they are produced (thymus for T cells, bone marrow for B cells). This is called central tolerance. An important, additional method of tolerance is through regulatory Th cells which inhibit autoreactive cells either directly or more likely through cytokines. Given that virtually all immune responses require initiation and regulation by T helper cells, a major aim of any tolerance induction regime would be to target these cells. Similarly, since Tc's are very important effector cells, their production is a major aim of strategies for, e.g., anti-cancer and anti-viral therapy.

The Thymus

The thymus is arguably the major organ in the immune system because it is the primary site of production of T lymphocytes. Its role is to attract appropriate bone marrow-derived precursor cells from the blood, and induce their commitment to the T cell lineage including the gene rearrangements necessary for the production of the T cell receptor for antigen (TCR). Associated with this is a remarkable degree of cell division to expand the number of T cells and hence increase the likelihood that every foreign antigen will be recognized and eliminated. A unique feature of T cell recognition of antigen, however, is that unlike B cells, the TCR only recognizes peptide fragments physically associated with MHC molecules; normally this is self MHC and this ability is selected for in the thymus. This process is called positive selection and is an exclusive feature of cortical epithelial cells. If the TCR fails to bind to the self MHC/peptide complexes, the T cell dies by “neglect”—it needs some degree of signalling through the TCR for its continued maturation.

While the thymus is fundamental for a functional immune system, releasing ˜1% of its T cell content into the bloodstream per day, one of the apparent anomalies of mammals is that this organ undergoes severe atrophy as a result of sex steroid production. This atrophy occurs gradually over ˜5-7 years; the nadir level of T cell output being reached around 20 years of age (Douek et al., 1998). Structurally the thymic atrophy involves a progressive loss of lymphocyte content, a collapse of the cortical epithelial network, an increase in extracellular matrix material and an infiltration of the gland with fat cells—adipocytes—and lipid deposits (Haynes et al., 1999). This can begin even in young (around the age of 5 years—Mackall et al., 1998) children but is profound from the time of puberty when sex steroid levels reach a maximum. For normal healthy individuals this loss of production and release of new T cells does not always have clinical consequences, although immune-based disorders such as general immunodeficiency and poor responsiveness to vaccines and an increase in the frequency of autoimmune diseases such as multiple sclerosis, rheumatoid arthritis and lupus (Doria et al., 1997; Weyand et al., 1998; Castle, 2000; Murasko et al., 2002) increase in incidence and severity with age. When there is a major loss of T cells, e.g., in AIDS and following chemotherapy or radiotherapy, the patients are highly susceptible to disease because all these conditions involve a loss of T cells (especially Th in HIV infections) or all blood cells including T cells in the case of chemotherapy and radiotherapy. As a consequence these patients lack the cells needed to respond to infections and they become severely immune suppressed (Mackall et al., 1995; Heitger et al., 2002).

Many T cells will develop, however, which can recognize by chance, with high affinity, self MHC/peptide complexes. Such T cells are thus potentially self-reactive and could cause severe autoimmune diseases such as multiple sclerosis, arthritis, diabetes, thyroiditis and systemic lupus erythematosis (SLE). Fortunately, if the affinity of the TCR to self MHC/peptide complexes is too high in the thymus, the developing thymocyte is induced to undergo a suicidal activation and dies by apoptosis, a process called negative selection. This is called central tolerance. Such T cells die rather than respond because in the thymus they are still immature. The most potent inducers of this negative selection in the thymus are APC called dendritic cells (DC). Being APC they deliver the strongest signal to the T cells; in the thymus this causes deletion, in the peripheral lymphoid organs where the T cells are more mature, the DC cause activation.

Thymus Atrophy

The thymus is influenced to a great extent by its bidirectional communication with the neuroendocrine system (Kendall, 1988). Of particular importance is the interplay between the pituitary, adrenals and gonads on thymic function including both trophic (thyroid stimulating hormone or TSH, and growth hormone or GH) and atrophic effects (leutinizing hormone or LH, follicle stimulating hormone or FSH, and adrenocorticotropic hormone or ACTH) (Kendall, 1988; Homo-Delarche, 1991). Indeed one of the characteristic features of thymic physiology is the progressive decline in structure and function which is commensurate with the increase in circulating sex steroid production around puberty which, in humans generally occurs from the age of 12-14 onwards (Hirokawa and Makinodan, 1975; Tosi et al., 1982 and Hirokawa, et al., 1994). The precise target of the hormones and the mechanism by which they induce thymus atrophy and improved immune responses has yet to be determined. Since the thymus is the primary site for the production and maintenance of the peripheral T cell pool, this atrophy has been widely postulated as the primary cause of an increased incidence of immune-based disorders in the elderly. In particular, deficiencies of the immune system illustrated by a decrease in T cell dependent immune functions such as cytolytic T cell activity and mitogenic responses, are reflected by an increased incidence of immunodeficiency such as increased general infections, autoimmune diseases such as multiple sclerosis, rheumatoid arthritis and Systemic Lupus Erythematosis (SLE). There is also an increase in cancerous tumor load in later life (Hirokawa, 1998; Doria et al., 1997; Castle, 2000).

The impact of thymus atrophy is reflected in the periphery, with reduced thymic input to the T cell pool resulting in a less diverse T cell receptor (TCR) repertoire. Altered cytokine profile (Hobbs et al., 1993; Kurashima et al., 1995), changes in CD4⁺ and CD8⁺ subsets and a bias towards memory as opposed to naïve T cells (Mackall et al., 1995) are also observed. Furthermore, the efficiency of thymopoiesis is impaired with age such that the ability of the immune system to regenerate normal T cell numbers after T cell depletion is eventually lost (Mackall et al., 1995). However, recent work by Douek et al. (1998) has shown presumably thymic output (as exemplified by the presence of T cells with T Cell Receptor Excision Circles (TRECs); TRECs are formed as part of the generation of the TCR for antigen and are only found in newly produced T cells) to occur even if only very slight (˜5% of the young levels), in older (e.g., even sixty-five years old and above) in humans. Excisional DNA products of TCR gene-rearrangement were used to demonstrate circulating, de novo produced naïve T cells after HIV infection in older patients. The rate of this output and subsequent peripheral T cell pool regeneration needs to be further addressed since patients who have undergone chemotherapy show a greatly reduced rate of regeneration of the T cell pool, particularly CD4⁺ T cells, in post-pubertal (at the time the thymus has reached substantial atrophy ˜25 years of age) patients compared to those who were pre-pubertal (prior to the increase in sex steroids in early teens (˜5-10 years of age)) (Mackall et al., 1995). This is further exemplified in recent work by Timm and Thoman (1999), who have shown that although CD4⁺ T cells are regenerated in old mice post bone marrow transplant (BMT), they appear to show a bias towards memory cells due to the aged peripheral microenvironment, coupled to poor thymic production of naïve T cells.

The thymus essentially consists of developing thymocytes interspersed within the diverse stromal cells (predominantly epithelial cell subsets) which constitute the microenvironment and provide the growth factors and cellular interactions necessary for the optimal development of the T cells. The symbiotic developmental relationship between thymocytes and the epithelial subsets that controls their differentiation and maturation (Boyd et al., 1993), means sex-steroid inhibition could occur at the level of either cell type which would then influence the status of the other. It is less likely that there is an inherent defect within the thymocytes themselves since previous studies, utilizing radiation chimeras, have shown that bone marrow (BM) stem cells are not affected by age (Hirokawa, 1998; Mackall and Gress, 1997) and have a similar degree of thymus repopulation potential as young BM cells. Furthermore, thymocytes in older aged animals (e.g., those≧18 months) retain their ability to differentiate to at least some degree (George and Ritter, 1996; Hirokawa et al., 1994; Mackall et al., 1998). However, recent work by Aspinall (1997) has shown a defect within the precursor CD3⁻CD4⁻CD8⁻ triple negative (TN) population occurring at the stage of TCRγ chain gene-rearrangement.

In the particular case for AIDS, the primary defect in the immune system is the destruction of CD4+ cells and to a lesser extent the cells of the myleoid lineages of macrophages and dendritic cells (DC). Without these the immune system is paralysed and the patient is extremely susceptible to opportunistic infection with death a common consequence. The present treatment for AIDS is based on a multitude of anti-viral drugs to kill or deplete the HIV virus. Such therapies are now becoming more effective with viral loads being reduced dramatically to the point where the patient can be deemed as being in remission. The major problem of immune deficiency still exists however, because there are still very few functional T cells, and those which do recover, do so very slowly. The period of immune deficiency is thus still a very long time and in some cases immune defence mechanisms may never recover sufficiently. The reason for this is that in post-pubertal people the thymus is atrophied.

Vaccines

Vaccination is the process of preparing an animal to respond to an antigen. Vaccination is more complex than immune recognition and involves not only B cells and cytotoxic T cells but other types of lymphoid cells as well. During vaccination, cells which recognize the antigen (B cells or cytotoxic T cells) are clonally expanded. In addition, the population of ancillary cells (helper T cells) specific for the antigen also increase. Vaccination also involves specialized antigen presenting cells which can process the antigen and display it in a form which can stimulate one of the two pathways.

Vaccines can be divided into two classes: those that provide active vaccination and those that provide passive vaccination. Passive vaccination involves the administration to a patient of antibodies from a heterologous source to react against foreign antigens in the patient or that the patient will encounter. Such vaccination is usually very short lived, as the native immune system of the patient is not involved. The present disclosure is in the field of active vaccinations, where an antigen is administered to a patient whose immune system then responds to the antigen by forming antibodies specific to the antigen. Vaccination may include both prophylactic and therapeutic vaccines.

One strategy for vaccinating against infection is through the use of killed or inactivated vaccines (e.g., by heat or other chemicals) to present agent's proteins to an individual's immune system. The administration of killed or inactivated agent to an individual causes presentation of the agent to the patient's immune system in a noninfective form and the individual then mounts an immune response against it. Killed or inactivated pathogen vaccines provide protection by directly generating T-helper and humoral immune responses against the antigens of the agent.

Another method of vaccinating against agents is to provide an attenuated vaccine. Attenuated vaccines are essentially live vaccines, which exhibit a reduced infectivity. Attenuated vaccines are often produced by passaging several generations of the pathogen through a permissive host until the progeny agents are no longer virulent. By maintaining a lower level of infectivity, the attenuated vaccine maintains a persistent, low level of infection, thereby eliciting protective T helper (Th), cytotoxic T lymphocyte (Tc or CTL), and humoral immune responses in the host. Examples of live attenuated vaccines include the poliovirus and smallpox vaccines.

Another method of vaccination is the use of recombinant vaccines. The first type of recombinant vaccine is an attenuated vaccine in which the agent (e.g., a virus) has specific virulence-causing genes deleted, which renders the virus non-virulent. The second type of recombinant vaccine employs the use of infective, but non-virulent, vectors which are genetically modified to insert a gene encoding target antigens. Examples of a recombinant vaccines is a vaccinia virus vaccines.

Yet another method of vaccination is the use of subunit vaccines. Subunit vaccines generally consist of at least one protein isolated from the agent, which are presented to the immune system. Typically, the proteins utilized in subunit vaccine are those displayed by the agent to that, upon infection, the patient's immune system is better able to mount a defense against it. Examples of subunit vaccines include the hepatitis B vaccine, in which hepatitis B surface antigen (HBsAg) is the agent-specific protein.

A final method of vaccination is a DNA vaccine. DNA-based vaccines generally use bacterial plasmids to express protein immunogens in vaccinated hosts. Recombinant DNA technology is used to clone cDNAs encoding immunogens of interest into eukaryotic expression vectors. Vaccine plasmids are then amplified in bacteria, purified, and directly inoculated into the hosts being vaccinated. DNA can be inoculated by a needle injection of DNA in saline, or by a gene gun device which delivers DNA-coated gold beads into the skin. Plasmid DNA is taken up by host cells, the vaccine protein is expressed, processed and presented in the context of self-MHC class I (MHC I) and MHC class II (MHC II) molecules, and an immune response against the DNA-encoded immunogen is generated.

Methods for preparation and use of such vaccines will be well-known to, or may be readily ascertained by, those of ordinary skill in the art.

There are several parameters that can influence the nature and extent of immune responses: the level and type of antigen, the site of vaccination, the availability of appropriate APC, the general health of the individual, and the status of the T and B cell pools. Of these, T cells are the most vulnerable because of the marked sex steroid-induced shutdown in thymic export that becomes profound from the onset of puberty. Any vaccination program should therefore only be logically undertaken when the level of potential responder T cells is optimal with respect to both the existence of naïve T cells representing a broad repertoire of specificity, and the presence of normal ratios of Th1 to Th2 cells and Th to Tc cells. The type of T cell help that supports an immune response determines whether the raised antibody will be C′-dependent and phagocyte-mediated defenses will be mobilized (a type 1 response), or whether the raised antibody will be C′-independent and phagocyte-independent defenses will be mobilized (a type 2 response) (for reviews, see Fearon and Locksley (1996); Seder and Paul (1994)). Each of these types of responses are associated with Th and CTL cells with distinctive patterns of lymphokine and chemokine expression that support specific rearrangements of immunoglobulin (Ig) genes, patterns of lymphocyte trafficking, and types of innate immune responses. Signature lymphokines and chemokines for Th1 cells include IFN-γ and RANTES, and for Th2 cells, IL-4 and eotaxin. Historically type 1 responses have been associated with the raising of cytotoxic T cells and type 2 responses with the raising of antibody. Thus, the level and type of cytokines generated may also be manipulated to be appropriate for the desired response (e.g., some diseases require Th1 responses, and some require Th2 responses, for protective immunity). This includes codelivery of Th1- or Th2-type cytokines (e.g., delivery of recombinant cytokines or DNA encoding cytokines) to shift the immune response patterns in the patient. Immunostimulatory CpG oligonucletides have also been utilized to shift immune response to various vaccine formulations to a more Th1-type response.

The ability to reactivate the atrophic thymus through inhibition of sex steroid production, for example at the level of leutinizing hormone releasing hormone (LHRH) signaling to the pituitary, provides a potent means of generating a new cohort of naïve T cells with a diverse repertoire of TCR types. This process effectively reverts the thymus to its pre-pubertal state, and does so by using the normal regulatory molecules and pathways which lead to optimal thymopoiesis.

### SUMMARY OF THE INVENTION

The present inventors have demonstrated that thymic atrophy (aged induced or as a consequence of conditions such as chemotherapy or radiotherapy) can be profoundly reversed by inhibition of sex steroid production, with virtually complete restoration of thymic structure and function. The present inventors have also found that the basis for this thymus regeneration is in part due to the initial expansion of precursor cells which are derived both intrathymically and via the blood stream. This finding suggests that is possible to seed the thymus with exogenous haemopoietic stem cells (HSC) which have been injected into the subject.

The ability to seed the thymus with genetically modified or exogenous HSC by disrupting sex steroid signalling to the thymus, means that gene therapy in the HSC may be used more efficiently to treat T cell (and myeloid cells which develop in the thymus) disorders. HSC stem cell therapy has met with little or no success to date because the thymus is dormant and incapable of taking up many if any HSC, with T cell production less than 1% of normal levels.

The present disclosure concerns methods for improving a patient's immune response to a vaccine. This is accomplished by quantitatively and qualitatively restoring the peripheral T cell pool, particularly at the level of naïve T cells. These naäve T cells are then able to respond to a greater degree to presented foreign antigen.

A patient's immune response to a vaccine may be improved by causing the patient's thymus to reactivate and the functional status of the peripheral T cells to be improved. In this instance, the thymus will begin to increase the rate of proliferation of the early precursor cells (CD3⁻CD4⁻CD8⁻ cells) and convert them into CD4⁺CD8⁺, and subsequently new mature CD3^{hi}CD4⁺CD8⁻ (T helper (Th) lymphocytes) or CD3^{hi} CD4⁻CD8⁺ (T cytotoxic lymphocytes (CTL)). The rejuvenated thymus will also take up new haemopoietic stem cells (HSC) from the blood stream and convert them into new T cells and intrathymic dendritic cells. The increased activity in the thymus resembles that found in a normal younger thymus (prior to the atrophy at ˜20 years of age) caused increased levels of sex steroids. The result of this renewed thymic output is increased levels of naïve T cells (those T cells which have not yet encountered antigen) in the blood. There is also an increase in the ability of the blood T cells to respond to stimulation, e.g., by using anti-CD28 Abs, cross-linking the TCR with, e.g., anti-CD3 antibodies, or stimulation with mitogens, such as pokeweed mitogen (PWM). This combination of events results in the body becoming better able to respond to vaccine antigens, thereby ultimately being able to better defend against infection and other immune system challenges (e.g., cancers), or becoming better able to recover from chemotherapy and radiotherapy.

In one embodiment, the patient may receive HSC transplantation.

In one embodiment the atrophic thymus in an aged (post-pubertal) patient is reactivated. This reactivated thymus becomes capable of taking up HSC and bone marrow cells from the blood and converting them in the thymus to new T cells and DC.

In one aspect the present disclosure provides a improving the vaccine response to a vaccine antigen (e.g., that of an agent), the method comprising disrupting sex steroid mediated signaling to the thymus in the patient. In one embodiment, GnRH analogs (agonist and antagonists thereto) are used to disrupt sex steroid-mediated signaling to the thymus. In another embodiment, GnRH analogs directly stimulate (i.e., directly increase the functional activity of) the thymus, bone marrow, and pre-existing cells of the immune system, such as T cells, B cells, and dendritic cells (DC).

The methods of this invention rely on blocking sex steroid mediated signaling to the thymus. In one embodiment, chemical castration is used. In another embodiment surgical castration is used. Castration reverses the state of the thymus to its pre-pubertal state, thereby reactivating it.

In a particular embodiment sex steroid mediated signaling to the thymus is blocked by the administration of agonists or antagonists of LHRH, anti-estrogen antibodies, anti-androgen antibodies, passive (antibody) or active (antigen) anti-LHRH vaccinations, or combinations thereof (“blockers” ).

In one embodiment, the blocker(s) is administered by a sustained peptide-release formulation. Examples of sustained peptide-release formulations are provided in WO 98/08533, the entire contents of which are incorporated herein by reference.

In one embodiment, bone marrow or HSC are also transplanted into the patient to provide a reservoir of precursor cells for the renewed thymic growth. These HSC have the capability of turning into DC, which may have the effect of providing better antigen presentation to the T cells and therefore a better immune response (e.g., increased antibody (Ab) production and effector T cells number and/or function). In one embodiment, the bone marrow or HSC are transplanted just before, at the time of, or after reactivation of the thymus, thereby creating a new population of T cells.

In another embodiment, the method comprises transplanting enriched HSC into the subject. The HSC may be autologous or heterologous.

In cases where the subject is infected with HIV, the HSC may be genetically modified such that they and their progeny, in particular T cells, macrophages and dendritic cells, are resistant to infection and/or destruction with the HIV virus. The genetic modification may involve introduction into HSC one or more nucleic acid molecules which prevent viral replication, assembly and/or infection. The nucleic acid molecule may be a gene which enclodes an antiviral protein, an antisense construct, a ribozyme, a dsRNA and a catalytic nucleic acid molecule.

In some embodiments, the subject has AIDS and has had the viral load reduced by anti-viral treatment. In a further embodiment, the subject is post-pubertal.

In certain embodiments, inhibition of sex steroid production is achieved by either castration or administration of a sex steroid analogue(s). Non-limiting sex steroid analogues include eulexin, goserelin, leuprolide, dioxalan derivatives such as triptorelin, meterelin, buserelin, histrelin, nafarelin, lutrelin, leuprorelin, and luteinizing hormone-releasing hormone analogues. In some embodiments, the sex steroid analogue is an analogue of luteinizing hormone-releasing hormone. In certain embodiments, the luteinizing hormone-releasing hormone analogue is deslorelin.

In cases where the subject has defective T cells, the HSC may be genetically modified to normalise the defect. For diseases such as T cell leukaemias, the modification may include the introduction of nucleic acid constructs or genes which normalize the HSC and inhibit or reduce its likelihood of becoming a cancer cell.

It will be appreciated by those skilled in the art that the present method may be useful in treating any T cell disorder which has a defined genetic basis. One method involves reactivating thymic function through inhibition of sex steroids to increase the uptake of blood-borne haemopoietic stem cells (HSC). In general, after the onset of puberty, the thymus undergoes severe atrophy under the influence of sex steroids, with its cellular production reduced to less than 1% of the pre-pubertal thymus. The present invention is based on the finding that the inhibition of production of sex steroids releases the thymic inhibition and allows a full regeneration of its function, including increased uptake of blood-derived HSC. The origin of the HSC can be directly from injection or from the bone marrow following prior injection. It is envisaged that blood cells derived from modified HSC will pass the genetic modification onto their progeny cells, including HSC derived from self-renewal, and that the development of these HSC along the T cell and dendritic cell lineages in the thymus is greatly enhanced if not fully facilitated by reactiving thymic function through inhibition of sex steroids.

The method of the present invention is particularly for treatment of AIDS, where the treatment preferably involves reduction of viral load, reactivation of thymic function through inhibition of sex steroids and transfer into the patients of HSC (autologous or from a second party donor) which have been genetically modified such that all progeny (especially T cells, DC) are resistant to further HIV infection. This means that not only will the patient be depleted of HIV virus and no longer susceptible to general infections because the T cells have returned to normal levels, but the new T cells being resistant to HIV will be able to remove any remnant viral infected cells. In principle a similar strategy could be applied to gene therapy in HSC for any T cell defect or any viral infection which targets T cells.

### DESCRIPTION OF THE FIGURES

FIG. 1A, 1B, and 1C: Castration rapidly regenerates thymus cellularity. FIG. 1A-1C show the changes in thymus weight and thymocyte number pre- and post-castration. Thymus atrophy results in a significant decrease in thymocyte numbers with age, as measured by thymus weight (FIG. 1A) or by the number of cells per thymus (FIGS. 1B and 1C). For these studies, aged (i.e., 2-year old) male mice were surgically castrated. Thymus weight in relation to body weight (FIG. 1A) and thymus cellularity (FIGS. 1B and 1C) were analyzed in aged (1 and 2 years) and at 2-4 weeks post-castration (post-cx) male mice. A significant decrease in thymus weight and cellularity was seen with age compared to young adult (2-month) mice. This decrease in thymus weight and cell number was restored by castration, although the decrease in cell number was still evident at 1 week post-castration (see FIG. 1C). By 2 weeks post-castration, cell numbers were found to increase to approximately those levels seen in young adults (FIGS. 1B and 1C). By 3 weeks post-castration, numbers have significantly increased from the young adult and these were stabilized by 4 weeks post-castration (FIGS. 1B and 1C). Results are expressed as mean±1SD of 4-8 mice per group (FIGS. 1A and 1B) or 8-12 mice per group (FIG. 1C). **=p≦0.01; ***=p≦0.001 compared to young adult (2 month) thymus and thymus of 2-6 wks post-castrate mice.

FIG. 2A-F: Castration restores the CD4:CD8 T cell ratio in the periphery. For these studies, aged (2-year old) mice were surgically castrated and analyzed at 2-6 weeks post-castration for peripheral lymphocyte populations. FIGS. 2A and 2B show the total lymphocyte numbers in the spleen. Spleen numbers remain constant with age and post-castration because homeostasis maintains total cell numbers within the spleen (FIGS. 2A and 2B). However, cell numbers in the lymph nodes in aged (18-24 months) mice were depleted (FIG. 2B). This decrease in lymph node cellularity was restored by castration (FIG. 2B). FIGS. 2C and 2D show that the ratio of B cells to T cells did not change with age or post-castration in either the spleen or lymph node, as no change in this ratio was seen with age or post-castration. However, a significant decrease (p<0.001) in the CD4+:CD8+ T cell ratio was seen with age in both the (pooled) lymph node and the spleen (FIGS. 2E and 2F). This decrease was restored to young adult (i.e., 2 month) levels by 4-6 weeks post-castration (FIGS. 2E and 2F). Results are expressed as mean±1SD of 4-8 (FIGS. 2A, 2C, and 2E) or 8-10 (FIGS. 2B, 2D, and 2F) mice per group. *=p≦0.05; **=p≦0.01; ***=p≦0.001 compared to young adult (2-month) and post-castrate mice.

FIG. 3: Thymocyte subpopulations are retained in similar proportions despite thymus atrophy or regeneration by castration. For these studies, aged (2-year old) mice were castrated and the thymocyte subsets analysed based on the markers CD4 and CD8. Representative Fluorescence Activated Cell Sorter (FACS) profiles of CD4 (X-axis) vs. CD8 (Y-axis) for CD4-CD8-DN, CD4+CD8+DP, CD4+CD8− and CD4-CD8+ SP thymocyte populations are shown for young adult (2 months), aged (2 years) and aged, post-castrate animals (2 years, 4 weeks post-cx). Percentages for each quadrant are given above each plot. No difference was seen in the proportions of any CD4/CD8 defined subset with age or post-castration. Thus, subpopulations of thymocytes remain constant with age and there was a synchronous expansion of thymocytes following castration.

FIG. 4: Regeneration of thymocyte proliferation by castration. Mice were injected with a pulse of BrdU and analysed for proliferating (BrdU⁺) thymocytes. FIGS. 4A and 4B show representative histograms of the total % BrdU⁺ thymocytes with age and post-cx. FIG. 4C shows the percentage (left graph) and number (right graph) of proliferating cells at the indicated age and treatment (e.g., week post-cx). For these studies, aged (2-year old) mice were castrated and injected with a pulse of bromodeoxyuridine (BrdU) to determine levels of proliferation. Representative histogram profiles of the proportion of BrdU+ cells within the thymus with age and post-castration are shown (FIGS. 4A and 4B). No difference was observed in the total proportion of proliferation within the thymus, as this proportion remains constant with age and following castration (FIGS. 4A, 4B, and left graph in FIG. 4C). However, a significant decrease in number of BrdU⁺ cells was seen with age (FIG. 4C, right graph). By 2 weeks post-castration, the number of BrdU⁺ cells increased to a number that similar to seen in young adults (i.e., 2 month) (FIG. 4C, right graph). Results are expressed as mean±1SD of 4-14 mice per group. ***=p≦0.001 compared to young adult (2-month) control mice and 2-6 weeks post-castration mice.

FIGS. 5A-K: Castration enhances proliferation within all thymocyte subsets. For these studies, aged (2-year old) mice were castrated and injected with a pulse of bromodeoxyuridine (BrdU) to determine levels of proliferation. Analysis of proliferation within the different subsets of thymocytes based on CD4 and CD8 expression within the thymus was performed. FIG. 5A shows that the proportion of each thymocyte subset within the BrdU+ population did not change with age or post-castration. However, as shown in FIG. 5B, a significant decrease in the proportion of DN (CD4−CD8−) thymocytes proliferating was seen with age. A decrease in the proportion of TN (i.e., CD3⁻CD4⁻CD8⁻) thymocytes was also seen with age (data not shown). Post-castration, this was restored and a significant increase in proliferation within the CD4−CD8+ SP thymocytes was observed. Looking at each particular subset of T cells, a significant decrease in the proportion of proliferating cells within the CD4−CD8− and CD4−CD8+ subsets was seen with age (FIGS. 5C and 5E). At 1 and 2 weeks post-castration, the percentage of BrdU+ cells within the CD4−CD8+ population was significantly increased above the young control group (FIG. 5E). FIG. 5F shows that no change in the total proportion of BrdU+ cells (i.e., proliferating cells) within the TN subset was seen with age or post-castration. However, a significant decrease in proliferation within the TN1 (CD44+CD25−CD3−CD4−CD8−) subset (FIG. 5H) and significant increase in proliferation within TN2 (CD44+CD25+CD3−CD4−CD8−) subset (FIG. 5I) was seen with age. This was restored post-castration (FIGS. 5G, 5H, and 5I). Results are expressed as mean±1SD of 4-17 mice per group. *=p≦0.05; **=p≦0.01 (significant); ***=p≦0.001 (highly significant) compared to young adult (2-month) mice; \hat{ }=significantly different from 1-6 weeks post-castrate mice (FIGS. 5C-5E) and 2-6 weeks post-castrate mice (FIGS. 5H-5K).

FIGS. 6A-6C: Castration increases T cell export from the aged thymnus. For these studies, aged (2-year old) mice were castrated and were injected intrathymically with FITC to determine thymic export rates. The number of FITC+ cells in the periphery was calculated 24 hours later. As shown in FIG. 6A, a significant decrease in recent thymic emigrant (RTE) cell numbers detected in the periphery over a 24 hours period was observed with age. Following castration, these values had significantly increased by 2 weeks post-cx. As shown in FIG. 6B, the rate of emigration (export/total thymus cellularity) remained constant with age, but was significantly reduced at 2 weeks post-castration. With age, a significant increase in the ratio of CD4⁺ to CD8⁺ RTE was seen; this was normalized by 1-week post-cx (FIG. 6C). Results are expressed as mean±1SD of 4-8 mice per group. *=p≦0.05; **=p≦0.01; ***=p≦0.001 compared to young adult (2-month) mice for (FIG. 6A) and compared to all other groups (FIGS. 6B and 6C). \hat{ }=p≦0.05 compared to aged (1- and 2-year old) non-cx mice and compared to 1-week post-cx, aged mice.

FIGS. 7A and 7B: Castration enhances thymocyte regeneration following T cell depletion. 3-month old mice were either treated with cyclophosphamide (intraperitoneal injection with 200 mg/kg body weight cyclophosphamide, twice over 2 days) (FIG. 7A) or exposed to sublethal irradiation (625 Rads) (FIG. 7B). For both models of T cell depletion studied, castrated (Cx) mice showed a significant increase in the rate of thymus regeneration compared to their sham-castrated (ShCx) counterparts. Analysis of total thymocyte numbers at 1 and 2-weeks post-T cell depletion (TCD) showed that castration significantly increases thymus regeneration rates after treatment with either cyclophosphamide or sublethal irradiation (FIGS. 7A and 7B, respectively). Data is presented as mean±1SD of 4-8 mice per group. For FIG. 7A, *** =p≦0.001 compared to control (age-matched, untreated) mice; \hat{ }=p≦0.001 compared to both groups of castrated mice. For FIG. 7B, ***=p≦0.001 compared to control mice; \hat{ }=p≦0.001 compared to mice castrated 1-week prior to treatment at 1-week post-irradiation and compared to both groups of castrated mice at 2-weeks post-irradiation.

FIGS. 8A-8C: Changes in thymus (FIG. 8A), spleen (FIG. 8B) and lymph node (FIG. 8C) cell numbers following treatment with cyclophosphamide and castration. For these studies, (3 month old) mice were depleted of lymphocytes using cyclophosphamide (intraperitoneal injection with 200 mg/kg body weight cyclophosphamide, twice over 2 days) and either surgically castrated or sham-castrated on the same day as the last cyclophosphamide injection. Thymus, spleen and lymph nodes (pooled) were isolated and total cellularity evaluated. As shown in FIG. 8A, significant increase in thymus cell number was observed in castrated mice compared to sham-castrated mice. Note the rapid expansion of the thymus in castrated animals when compared to the non-castrate (cyclophosphamide alone) group at 1 and 2 weeks post-treatment. FIG. 8B shows that castrated mice also showed a significant increase in spleen cell number at 1-week post-cyclophosphamide treatment. A significant increase in lymph node cellularity was also observed with castrated mice at 1-week post-treatment (FIG. 8C). Thus, spleen and lymph node numbers of the castrate group were well increased compared to the cyclophosphamide alone group at one week post-treatment. By 4 weeks, cell numbers are normalized. Results are expressed as mean±1SD of 3-8 mice per treatment group and time point. =p≦0.001 compared to castrated mice.

FIGS. 9A-B: Total lymphocyte numbers within the spleen and lymph nodes post-cyclophosphamide treatment. Sham-castrated mice had significantly lower cell numbers in the spleen at 1 and 4-weeks post-treatment compared to control (age-matched, untreated) mice (FIG. 9A). A significant decrease in cell number was observed within the lymph nodes at 1 week post-treatment for both treatment groups (FIG. 9B). At 2-weeks post-treatment, Cx mice had significantly higher lymph node cell numbers compared to ShCx mice (FIG. 9B). Each bar represents the mean±1SD of 7-17 mice per group. *=p≦0.05; **=p≦0.01 compared to control (age-matched, untreated). \hat{ }p≦0.05 compared to castrate mice.

FIG. 10: Changes in thymus (open bars), spleen (gray bars) and lymph node (black bars) cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIGS. 11A-C: Changes in thymus (FIG. 11A), spleen (FIG. 11B) and lymph node (FIG. 11C) cell numbers following irradiation (625 Rads) one week after surgical castration. For these studies, young (3-month old) mice were depleted of lymphocytes using sublethal (625 Rads) irradiation. Mice were either sham-castrated or castrated 1-week prior to irradiation. A significant increase in thymus regeneration (i.e., faster rate of thymus regeneration) was observed with castration (FIG. 11A). Note the rapid expansion of the thymus in castrated animals when compared to the non-castrate (irradiation alone) group at 1 and 2 weeks post-treatment. (n=3-4 per treatment group and time point). No difference in spleen (FIG. 11B) or lymph node (FIG. 11C) cell numbers was seen with castrated mice. Lymph node cell numbers were still chronically low at 2-weeks post-treatment compared to control mice (FIG. 11C). Results are expressed as mean±1SD of 4-8 mice per group. *=p≦0.05; **=p≦0.01 compared to control mice; ***=p≦0.001 compared to control and castrated mice.

FIGS. 12A-C: Changes in thymus (FIG. 12A), spleen (FIG. 12B) and lymph node (FIG. 12C) cell numbers following irradiation and castration on the same day. For these studies, young (3-month old) mice were depleted of lymphocytes using sublethal (625 Rads) irradiation. Mice were either sham-castrated or castrated on the same day as irradiation. Castrated mice showed a significantly faster rate of thymus regeneration compared to sham-castrated counterparts (FIG. 12A). Note the rapid expansion of the thymus in castrated animals when compared to the non-castrate group at 2 weeks post-treatment. No difference in spleen (FIG. 12B) or lymph node (FIG. 12C) cell numbers was seen with castrated mice. Lymph node cell numbers were still chronically low at 2-weeks post-treatment compared to control mice (FIG. 12C). Results are expressed as mean±1SD of 4-8 mice per group. *=p≦0.05; **=p≦0.01 compared to control mice; ***=p≦0.001 compared to control and castrated mice.

FIG. 13A-13B: Total lymphocyte numbers within the spleen and lymph nodes post-irradiation treatment. 3-month old mice were either castrated or sham-castrated 1-week prior to sublethal irradiation (625 Rads). Severe lymphopenia was evident in both the spleen (FIG. 13A) and (pooled) lymph nodes (FIG. 13B) at 1-week post-treatment. Splenic lymphocyte numbers were returned to control levels by 2-weeks post-treatment (FIG. 13A), while lymph node cellularity was still significantly reduced compared to control (age-matched, untreated) mice (FIG. 13B). No differences were observed between the treatment groups. Each bar represents the mean±1SD of 6-8 mice per group. **=p≦0.01; ***=p≦0.001 compared to control mice.

FIGS. 14A and 14B: FIG. 14A shows the lymph node cellularity following foot-pad immunization with Herpes Simplex Virus-1 (HSV-1). Note the increased cellularity in the aged post-castration as compared to the aged non-castrated group. FIG. 14B illustrates the overall activated cell number as gated on CD25 vs. CD8 cells by FACS (i.e., the activated cells are gated on CD8+CD25+ cells).

FIGS. 15A-15C: Vβ10 expression (HSV-specific) on CTL (cytotoxic T lymphocytes) in activated LN (lymph nodes) following HSV-1 inoculation. Despite the normal Vβ10 responsiveness in aged (i.e., 18 months) mice overall, in some mice a complete loss of Vβ10 expression was observed. Representative histogram profiles are shown. Note the diminution of a clonal response in aged mice and the reinstatement of the expected response post-castration.

FIG. 16: Castration restores responsiveness to HSV-1 immunisation. Mice were immunized in the hind foot-hock with 4×10⁵ pfu of HSV. On Day 5 post-infection, the draining lymph nodes (popliteal) were analysed for responding cells. Aged mice (i.e., 18 months-2 years, non-cx) showed a significant reduction in total lymph node cellularity post-infection when compared to both the young and post-castrate mice. Results are expressed as mean±1SD of 8-12 mice. **=p≦0.01 compared to both young (2-month) and castrated mice.

FIGS. 17A-B: Castration enhances activation following HSV-1 infection. FIG. 17A shows representative FACS profiles of activated (CD8⁺ CD25⁺) cells in the LN of HSV-1 infected mice. No difference was seen in proportions of activated CTL with age or post-castration. As shown in FIG. 17B, the decreased cellularity within the lymph nodes of aged mice was reflected by a significant decrease in activated CTL numbers. Castration of the aged mice restored the immune response to HSV-1 with CTL numbers equivalent to young mice. Results are expressed as mean±1SD of 8-12 mice. **=p≦0.01 compared to both young (2-month) and castrated mice.

FIG. 18: Specificity of the immune response to HSV-1. Popliteal lymph node cells were removed from mice immunised with HSV-1 (removed 5 days post-HSV-1 infection), cultured for 3-days, and then examined for their ability to lyse HSV peptide pulsed EL 4 target cells. CTL assays were performed with non-immunised mice as control for background levels of lysis (as determined by ⁵¹Cr-release). Aged mice showed a significant (p≦0.01, **) reduction in CTL activity at an E:T ratio of both 10:1 and 3:1 indicating a reduction in the percentage of specific CTL present within the lymph nodes. Castration of aged mice restored the CTL response to young adult levels since the castrated mice demonstrated a comparable response to HSV-1 as the young adult (2-month) mice. Results are expressed as mean of 8 mice, in triplicate ±1 SD. **=p≦0.01 compared to young adult mice; \hat{ }=significantly different to aged control mice (p≦0.05 for E:T of 3:1; p≦0.01 for E:T of 0.3:1).

FIGS. 19A and B: Analysis of Vβ TCR expression and CD4⁺ T cells in the immune response to HSV-1. Popliteal lymph nodes were removed 5 days post-HSV-1 infection and analysed ex-vivo for the expression of CD25, CD8 and specific TCRVβ markers (FIG. 19A) and CD4/CD8 T cells (FIG. 19B). The percentage of activated (CD25⁺) CD8⁺ T cells expressing either Vβ10 or Vβ8.1 is shown as mean±1SD for 8 mice per group in FIG. 19A. No difference was observed with age or post-castration. However, a decrease in CD4/CD8 ratio in the resting LN population was seen with age (FIG. 19B). This decrease was restored post-castration. Results are expressed as mean±1SD of 8 mice per group. ***=p≦0.001 compared to young and post-castrate mice.

FIGS. 20A-D: Castration enhances regeneration of the thymus (FIG. 20A, spleen (FIG. 20B) and bone marrow (FIG. 20D), but not lymph node (FIG. 20C) following bone marrow transplantation (BMT) of Ly5 congenic mice. 3 month old, young adults, C57/BL6 Ly5.1+ (CD45.1+) mice were irradiated (at 6.25 Gy), castrated, or sham-castrated 1 day prior to transplantation with C57/BL6 Ly5.2+ (CD45.2+) adult bone marrow cells (10⁶ cells). Mice were killed 2 and 4 weeks later and the), thymus (FIG. 20A), spleen (FIG. 20B), lymph node (FIG. 20C) and BM (FIG. 20D) were analysed for immune reconstitution. Donor/Host origin was determined with anti-CD45.2 (Ly5.2), which only reacts with leukocytes of donor origin. There were significantly more donor cells in the thymus of castrated mice 2 and 4 weeks after BMT compared to sham-castrated mice (FIG. 20A). Note the rapid expansion of the thymus in castrated animals when compared to the non-castrate group at all time points post-treatment. There were significantly more cells in these spleen and BM of castrated mice 2 and 4 weeks after BMT compared to sham-castrated mice (FIGS. 20B and 20D). There was no significant difference in lymph node cellularity 2, 4, and 6 weeks after BMT (FIG. 20C). Castrated mice had significantly increased congenic (Ly5.2) cells compared to non-castrated animals (data not shown). Data is expressed as mean±1SD of 4-5 mice per group. *=p≦0.05; **=p≦0.01.

FIGS. 21A and 21B: Changes in thymus cell number in castrated and noncastrated mice after fetal liver (E14, 10⁶ cells) reconstitution. (n=3-4 for each test group.) FIG. 21A shows that at two weeks, thymus cell number of castrated mice was at normal levels and significantly higher than that of noncastrated mice (*p≦0.05). Hypertrophy was observed in thymuses of castrated mice after four weeks. Noncastrated cell numbers remain below control levels. FIG. 21B shows the change in the number of CD45.2⁺ cells. CD45.2+ (Ly5.2+) is a marker showing donor derivation. Two weeks after reconstitution, donor-derived cells were present in both castrated and noncastrated mice. Four weeks after treatment approximately 85% of cells in the castrated thymus were donor-derived. There were no or very low numbers of donor-derived cells in the noncastrated thymus.

FIG. 22: FACS profiles of CD4 versus CD8 donor derived thymocyte populations after lethal irradiation and fetal liver reconstitution, followed by surgical castration. Percentages for each quadrant are given to the right of each plot. The age matched control profile is of an eight month old Ly5.1 congenic mouse thymus. Those of castrated and noncastrated mice are gated on CD45.2⁺ cells, showing only donor derived cells. Two weeks after reconstitution, subpopulations of thymocytes do not differ proportionally between castrated and noncastrated mice demonstrating the homeostatic thymopoiesis with the major thymocyte subsets present in normal proportions.

FIGS. 23A and 23B: Castration enhances dendritic cell generation in the thymus following fetal liver reconstitution. Myeloid and lymphoid dendritic cell (DC) number in the thymus after lethal irradiation, fetal liver reconstitution and castration. (n=3-4 mice for each test group.) Control (white) bars on the graphs are based on the normal number of dendritic cells found in untreated age matched mice. FIG. 23A shows donor-derived myeloid dendritic cells. Two weeks after reconstitution, donor-derived myeloid DC were present at normal levels in noncastrated mice. There were significantly more myeloid DC in castrated mice at the same time point. (*p≦0.05). At four weeks myeloid DC number remained above control levels in castrated mice. FIG. 23B shows donor-derived lymphoid dendritic cells. Two weeks after reconstitution, donor-derived lymphoid DC numbers in castrated mice were double those of noncastrated mice. Four weeks after treatment, donor-derived lymphoid DC numbers remained above control levels.

FIGS. 24A and 24B: Changes in total and donor CD45.2⁺ bone marrow cell numbers in castrated and noncastrated mice after fetal liver reconstitution. n=3-4 mice for each test group. FIG. 24A shows the total number of bone marrow cells. Two weeks after reconstitution, bone marrow cell numbers had normalized and there was no significant difference in cell number between castrated and noncastrated mice. Four weeks after reconstitution, there was a significant difference in cell number between castrated and noncastrated mice (*p≦0.05). Indeed, four weeks after reconstitution, cell numbers in castrated mice were at normal levels. FIG. 24B shows the number of CD45.2⁺ cells (i.e., donor-derived cells). There was no significant difference between castrated and noncastrated mice with respect to CD45.2+ cell number in the bone marrow two weeks after reconstitution. CD45.2⁺ cell number remained high in castrated mice at four weeks; however, there were no donor-derived cells in the noncastrated mice at the same time point. The difference in BM cellularity was predominantly due to a lack of donor-derived BM cells at 4-weeks post-reconstitution in sham-castrated mice. Data is expressed as mean±1SD of 3-4 mice per group. *=p≦0.05.

FIGS. 25A-25C: Changes in T cells and myeloid and lymphoid derived dendritic cells (DC) in bone marrow of castrated and noncastrated mice after fetal liver reconstitution. (n=3-4 mice for each test group.) Control (white) bars on the graphs are based on the normal number of T cells and dendritic cells found in untreated age matched mice. FIG. 25A shows the number of donor-derived T cells. As expected, numbers were reduced compared to normal T cell levels two and four weeks after reconstitution in both castrated and noncastrated mice. By 4 weeks there was evidence of donor-derived T cells in the castrated but not control mice. FIG. 25B shows the number of donor-derived myeloid dendritic cells (i.e., CD45.2+). Two weeks after reconstitution, donor myeloid DC cell numbers were normal in both castrated and noncastrated mice. At this time point there was no significant difference between numbers in castrated and noncastrated mice. However, by 4 weeks post-reconstitution, only the castrated animals have donor-derived myeloid dendritic cells. FIG. 25C shows the number of donor-derived lymphoid dendritic cells. Numbers were at normal levels two and four weeks after reconstitution for castrated mice but by 4 weeks there were no donor-derived DC in the sham-castrated group.

FIGS. 26A and 26B: Changes in total and donor (CD45.2⁺) lymph node cell numbers in castrated and non-castrated mice after fetal liver reconstitution. Control (striped) bars on the graphs are based on the normal number of lymph node cells found in untreated age matched mice. As shown in FIG. 26A, two weeks after reconstitution, cell numbers in the lymph node were not significantly different between castrated and sham-castrated mice. Four weeks after reconstitution, lymph node cell numbers in castrated mice were at control levels. FIG. 26B shows that there was no significant difference between castrated and non-castrated mice with respect to donor-derived CD45.2⁺ cell number in the lymph node two weeks after reconstitution. CD45.2+ cell numbers remained high in castrated mice at four weeks. There were no donor-derived cells in the non-castrated mice at the same point. Data is expressed as mean±1SD of 3-4 mice per group.

FIGS. 27A and 27B: Change in total and donor (CD45.2⁺) spleen cell numbers in castrated and non-castrated mice after fetal liver reconstitution. Control (white) bars on the graphs are based on the normal number of spleen cells found in untreated age matched mice. As shown in FIG. 27A, two weeks after reconstitution, there was no significant difference in the total cell number in the spleens of castrated and non-castrated mice. Four weeks after reconstitution, total cell numbers in the spleen were still approaching normal levels in castrated mice but were very low in non-castrated mice. FIG. 27B shows the number of donor (CD45.2⁺) cells. There was no significant difference between castrated and non-castrated mice with respect to donor-derived cells in the spleen, two weeks after reconstitution. However, four weeks after reconstitution, CD45.2⁺ cell number remained high in the spleens of castrated mice, but there were no donor-derived cells in the noncastrated mice at the same time point. Data is expressed as mean±1SD of 3-4 mice per group. *=p≦0.05

FIGS. 28A-28C: Castration enhances DC generation in the spleen after fetal liver reconstitution. Control (white) bars on the graphs are based on the normal number of splenic T cells and dendritic cells found in untreated age matched mice. As shown in FIG. 28A, total T cell numbers were reduced in the spleen two and four weeks after reconstitution in both castrated and sham-castrated mice. FIG. 28B shows that at 2-weeks post- reconstitution, donor-derived (CD45.2+) myeloid DC numbers were normal in both castrated and sham-castrated mice. Indeed, at two weeks there was no significant difference between numbers in castrated and non-castrated mice. However, no donor-derived DC were evident in sham-castrated mice at 4-weeks post-reconstitution, while donor-derived (CD45.2+) myeloid DC were seen in castrated mice. As shown in FIG. 28C, donor-derived lymphoid DC were also at normal levels two weeks after reconstitution. At two weeks there was no significant difference between numbers in castrated and non-castrated mice. Again, no donor-derived lymphoid DC were seen in sham-cx mice at 4-weeks compared to cx mice. Data is expressed as mean-±1 SD of 3-4 mice per group. *=p≦0.05.

FIGS. 29A-29C: Changes in T cells and myeloid and lymphoid derived dendritic cells (DC) in the mesenteric lymph nodes of castrated and non-castrated mice after fetal liver reconstitution. (n=3-4 mice for each test group.) Control (striped) bars are the number of T cells and dendritic cells found in untreated age matched mice. Mesenteric lymph node T cell numbers were reduced two and four weeks after reconstitution in both castrated and noncastrated mice (FIG. 29A). Donor derived myeloid dendritic cells were normal in the mesenteric lymph node of both castrated and noncastrated mice, while at four weeks they were decreased (FIG. 29B). At two weeks there was no significant difference between numbers in castrated and noncastrated mice. FIG. 29C shows donor-derived lymphoid dendritic cells in the mesenteric lymph node of both castrated and noncastrated mice. Numbers were at normal levels two and four weeks after reconstitution in castrated mice but were not evident in the control mice.

FIGS. 30A-30C: Castration Increases Bone Marrow and Thymic Cellularity following Congenic BMT. As shown in FIG. 30A, there are significantly more cells in the BM of castrated mice 2 and 4 weeks after BMT. BM cellularity reached untreated control levels (1.5×10⁷±1.5×10⁶) in the sham-castrates by 2 weeks. BM cellularity is above control levels in castrated mice 2 and 4 weeks after congenic BMT. FIG. 30b shows that there are significantly more cells in the thymus of castrated mice 2 and 4 weeks after BMT. Thymus cellularity in the sham-castrated mice is below untreated control levels (7.6×10⁷±5.2×10⁶) 2 and 4 weeks after congenics BMT. 4 weeks after congenic BMT and castration thymic cellularity is increased above control levels. FIG. 30C shows that there is no significant difference in splenic cellularity 2 and 4 weeks after BMT. Spleen cellularity has reached control levels (8.5×10⁷±1.1×10⁷) in sham-castrated and castrated mice by 2 weeks. Each group contains 4 to 5 animals. □ indicates sham-castration; ▪, castration.

FIG. 31: Castration increases the proportion of Haemopoietic Stem Cells following Congenic BMT. There is a significant increase in the proportion of donor-derived HSCs following castration, 2 and 4 weeks after BMT.

FIGS. 32A and 32B: Castration increases the proportion and number of Haemopoietic Stem Cells following Congenic BMT. As shown in FIG. 32A, there was a significant increase in the proportion of HSCs following castration, 2 and 4 weeks after BMT (*p≦0.05). FIG. 32B shows that the number of HSCs is significantly increased in castrated mice compared to sham-castrated controls, 2 and 4 weeks after BMT (*p≦0.05 **p≦0.01). Each group contains 4 to 5 animals. □ indicates sham-castration; ▪, castration.

FIGS. 33A and 33B: There are significantly more donor-derived B cell precursors and B cells in the BM of castrated mice following BMT. As shown in FIG. 33A, there were significantly more donor-derived CD45.1⁺B220⁺ IgM⁻ B cell precursors in the bone marrow of castrated mice compared to the sham-castrated controls (*p<0.05). FIG. 33B shows that there were significantly more donor-derived B220⁺ IgM⁺ B cells in the bone marrow of castrated mice compared to the sham-castrated controls (*p<0.05). Each group contains 4 to 5 animals. □ indicates sham-castration; ▪, castration.

FIG. 34: Castration does not effect the donor-derived thymocyte proportions following congenic BMT. 2 weeks after sham-castration and castration there is an increase in the proportion of donor-derived double negative (CD45.1⁺ CD4⁻CD8⁻) early thymocytes. There are very few donor-derived (CD45.1⁺) CD4 and CD8 single positive cells at this early time point. 4 weeks after BMT, donor-derived thymocyte profiles of sham-castrated and castrated mice are similar to the untreated control.

FIG. 35: Castration does not increase peripheral B cell proportions following congenic BMT. There is no difference in splenic B220 expression comparing castrated and sham-castrated mice, 2 and 4 weeks after congenic BMT.

FIG. 36: Castration does not increase peripheral B cell numbers following congenics BMT. There is no significant difference in B cell numbers 2 and 4 weeks after BMT. 2 weeks after congenic BMT B cell numbers in the spleen of sham-castrated and castrated mice are approaching untreated control levels (5.0×10⁷±4.5×10⁶). Each group contains 4 to 5 animals. □ indicates sham-castration; ▪, castration

FIG. 37: Donor-derived Triple negative, double positive and CD4 and CD8 single positive thymocyte numbers are increased in castrated mice following BMT. FIG. 37A shows that there were significantly more donor-derived triple negative (CD45.1⁺ CD3⁻CD4⁻CD8⁻) thymocytes in the castrated mice compared to the sham-castrated controls 2 and 4 weeks after BMT (*p<0.05 **p<0.01). FIG. 37B shows there were significantly more double positive (CD45.1⁺CD4⁺CD8⁺) thymocytes in the castrated mice compared to the sham-castrated controls 2 and 4 weeks after BMT (*p<0.05 **p<0.01). As shown in FIG. 37C, there were significantly more CD4 single positive (CD45.1⁺CD3⁺CD4⁺CD8⁻) thymocytes in the castrated mice compared to the sham-castrated controls 2 and 4 weeks after BMT (*p<0.05 **p<0.01). FIG. 37D shows there were significantly more CD8 single positive (CD45.1⁺CD3⁺CD4⁻CD8⁺) thymocytes in the castrated mice compared to the sham-castrated controls 4 weeks after BMT (*p<0.05 **p≦0.01). Each group contains 4 to 5 animals. □ indicates sham-castration; ▪, castration.

FIG. 38: There are very few donor-derived, peripheral T cells 2 and 4 weeks after congenic BMT. As shown in FIG. 38A, there was a very small proportion of donor-derived CD4⁺ and CD8⁺ T cells in the spleens of sham-castrated and castrated mice 2 and 4 weeks after congenic BMT. FIG. 38B shows that there was no significant difference in donor-derived T cell numbers 2 and 4 weeks after BMT. 4 weeks after congenics BMT there are significantly less CD4⁺ and CD8⁺ T cells in both sham-castrated and castrated mice compared to untreated age-matched controls (CD⁴⁺−1.1×10⁷±1.4×10⁶, CD8⁺−6.0×10⁶±1.0×10⁵) Each group contains 4 to 5 animals. □ indicates sham-castration; ▪, castration.

FIG. 39: Castration increases the number of donor-derived dendritic cells in the thymus 4 weeks after congenics BMT. As shown in FIG. 39A, donor-derived dendritic cells were CD45.1⁺CD11c⁺MHCII⁺. FIG. 39B shows there were significantly more donor-derived thymic DCs in the castrated mice 4 weeks after congenic BMT (*p<0.05). Dendritic cell numbers are at untreated control levels 2 weeks after congenic BMT (1.4×10⁵±2.8×10⁴). 4 weeks after congenic BMT dendritic cell numbers are above control levels in castrated mice. Each group contains 4 to 5 animals. □ indicates sham-castration; ▪, castration.

FIG. 40: The phenotypic composition of peripheral blood lymphocytes was analyzed in human patients (all>60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4 T cells. A less distinctive trend was seen within the CD8⁺ subset with 4/9 patients showing increased levels, albeit generally to a smaller extent than CD4⁺ T cells.

FIG. 41: Analysis of human patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 42: Analysis of the proportions of B cells and myeloid cells (NK, NKT and macrophages) within the peripheral blood of human patients undergoing LHRH agonist treatment demonstrated a varying degree of change within subsets. While NK, NKT and macrophage proportions remained relatively constant following treatment, the proportion of B cells was decreased in 4/9 patients.

FIG. 43: Analysis of the total cell numbers of B and myeloid cells within the peripheral blood of human patients post-treatment showed clearly increased levels of NK (5/9 patients), NKT (4/9 patients) and macrophage (3/9 patients) cell numbers post-treatment. B cell numbers showed no distinct trend with 2/9 patients showing increased levels; 4/9 patients showing no change and 3/9 patients showing decreased levels.

FIGS. 44A and 44B: The major change seen post-LHRH agonist treatment was within the T cell population of the peripheral blood. White bars represent pre-treatment; black bars represent 4 months post-LHRH-A treatment. Shown are representative FACS histograms (using four color staining) from a single patient. In particular there was a selective increase in the proportion of naïve (CD45RA⁺) CD4+ cells, with the ratio of naïve (CD45RA⁺) to memory (CD45RO⁺) in the CD4⁺ T cell subset increasing in 6/9 of the human patients.

### DETAILED DESCRIPTION OF THE INVENTION

The patent and scientific literature referred to herein establishes knowledge that is available to those with skill in the art. The issued U.S. patents, allowed applications, published foreign applications, and references, including GenBank database sequences, that are cited herein are hereby incorporated by reference to the same extent as if each was specifically and individually indicated to be incorporated by reference.

### Definitions

The phrase “modifying the T cell population makeup” refers to altering the nature and/or ratio of T cell subsets defined functionally and by expression of characteristic molecules. Examples of these characteristic molecules include, but are not limited to, the T cell receptor, CD4, CD8, CD3, CD25, CD28, CD44, CD62L and CD69.

The phrase “increasing the number of T cells” refers to an absolute increase in the number of T cells in a subject in the thymus and/or in circulation and/or in the spleen and/or in the bone marrow and/or in peripheral tissues such as lymph nodes, gastrointestinal, urogenital and respiratory tracts. This phrase also refers to a relative increase in T cells, for instance when compared to B cells.

A “subject having a depressed or abnormal T cell population or function” includes an individual infected with the human immunodeficiency virus, especially one who has AIDS, or any other, virus or infection which attacks T cells or any T cell disease for which a defective gene has been identified.

Furthermore, this phrase includes any post-pubertal individual, especially an aged person who has decreased immune responsiveness and increased incidence of disease as a consequence of post-pubertal thymic atrophy.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The present disclosure comprises methods for improving vaccine responses in a patient. This is accomplished by quantitatively and qualitatively restoring the peripheral T cell pool, particularly at the level of naïve T cells. Naïve T cells are those that have not yet contacted antigen and therefore have broad based specificity, i.e., are able to respond to any one of a wide variety of antigens. As a result of the procedures of this invention a large pool of naïve T cells becomes available to respond to antigen administered in a vaccine.

As described above, the aged (post-pubertal) thymus causes the body's immune system to function at less than peak levels (such as that found in the young, pre-pubertal thymus). The present disclosure uses reactivation of the thymus to improve immune system function, as exemplified by increased functionality of T lymphocytes (e.g., Th and CTL) including, but not limited to, better killing of target cells; increased release of cytokines, interleukins and other growth factors; increased levels of Ab in the plasma; and increased levels of innate immunity (e.g., natural killer (NK) cells, DC, neutrophils, macrophages, etc.) in the blood, all of which can be beneficial in increasing the response to vaccine antigens.

To generate new T lymphocytes, the thymus requires precursor cells; these can be derived from within the organ itself for a short time, but by 3-4 weeks, such cells are depleted and new hematopoietic stem cells (HSC) must be taken in (under normal circumstances this would be from the bone marrow via the blood). However, even in a normal functional young thymus, the intake of such cells is very low (sufficient to maintain T cell production at homeostatically regulated levels. Indeed the entry of cells into the thymus is extremely limited and effectively restricted to HSC (or at least prothymocytes which already have a preferential development along the T cell lineage). In the case of the thymus undergoing rejuvenation due a loss of sex steroid inhibition, this organ has been demonstrated to now be very receptive to new precursor cells circulating in the blood, such that the new T cells which develop from both intrathymic and external precursors. By increasing the level of the blood precursor cells, the T cells derived from them will progressively dominate the T cell pool. This means that any gene introduced into the precursors (HSC) will be passed onto all progeny T cells and eventually be present in virtually all of the T cell pool. The level of dominance of these cells over those derived from endogenous host HSC can be easily increased to very high levels by simply increasing the number of transferred exogenous HSC.

In one embodiment, blocking sex steroid mediated signaling to the thymus creates this pool of naïve T cells by reactivating the atrophied thymus. This disruption reverses the hormonal status of the recipient. One method for creating disruption is through castration. Methods for castration include but are not limited to chemical castration and surgical castration.

In another embodiment, the patient's thymus is reactivated following a subcutaneous injection of a “depot” or “impregnated implant” containing about 30 mg of Lupron. A 30 mg Lupron injection is sufficient for 4 months of sex steroid ablation to allow the thymus to rejuvenate and export new naïve T cells into the bloodstream. The length of time of the GnRH treatment will vary with the degree of thymic atrophy and damage, and will be readily determinably by those skilled in the art without undue experimentation. For example, the older the patient, or the more the patient has been exposed to T cell depleting reagents such as chemotherapy or radiotherapy, the longer it is likely that they will require GnRH. Four months is generally considered long enough to detect new T cells in the blood.

Methods of detecting new T cells in the blood are known in the art. For instance, one method of T cell detection is by determining the existence of T cell receptor excision circles (TREC's), which are formed when the TCR is being formed and are lost in the cell after it divides. Hence, TREC's are only found in new (naïve) T cells. TREC levels are one indicator of thymic function in humans. These and other methods are described in detail in WO/00 230,256.

In one embodiment, a person has already contacted the agent, or is at a high risk of doing so. The person may be given GnRH to activate their thymus, and also to improve their bone marrow function, which includes the increased ability to take up and produce HSC. The person may be injected with their own HSC, or may be injected with HSC from an appropriate donor, which has, e.g., treatment with G-CSF for 3 days (2 injections, subcutaneously per day) followed by collection of HSC from the blood on days 4 and 5. Following injection into the patient, the HSC enter the bone marrow and eventually evolve into antigen presenting cells (APC) throughout the body. Vaccine antigens are expressed in the context of MHC class I and/or MHC class II molecules on the surface of these APC. By expressing the desired vaccine antigen, the APC improve the activation of T and B lymphocytes.

As used herein, the terms “vaccinating,” “vaccination,” “vaccine,” “immunizing,” “immunization,” are related to the process of preparing a patient to respond to an antigen of an agent. Vaccination may include both prophylactic and therapeutic vaccines.

As used herein, “infectious agents,” “foreign agents,” and “agents” are used interchangeably and include any cause of disease in an individual. Agents include, but are not limited to viruses, bacteria, fungi, parasites, prions, cancers, allergens, asthma-inducing agents, “self” proteins and antigens which cause autoimmune disease, etc.

In one embodiment, the agent is a virus, bacteria, fungi, or parasite e.g., from the coat protein of a human papilloma virus (HPV), which causes uterine cancer; or an influenza peptide (e.g., hemagglutinin (HA), nucleoprotein (NP), or neuraminidase (N)).

Examples of infectious viruses include: Retroviridae (e.g., human immunodeficiency viruses, such as HIV-1 (also referred to as HTLV-III, LAV or HTLV-III/LAV), or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses, severe acute respiratory syndrome (SARS) virus); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (e.g, Hepatitis B virus); Parvoviridae (parvoviruses); Papovaviridae (papilloma viruses, polyoma viruses); Adenoviridae (most adenoviruses); Herpesviridae (e.g., herpes simplex virus (HSV) 1 and 2, varicella zoster virus, cytomegalovirus (CMV), herpes viruses); Poxviridae (e.g., variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class l=internally transmitted; class 2=parenterally transmitted (i.e., Hepatitis C); Norwalk and related viruses, and astroviruses).

Examples of infectious bacteria include: Helicobacter pyloris, Borelia burgdorferi, Legionella pneumophilia, Mycobacteria sporozoites (sp.) (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans.

Other infectious organisms (i.e., protists) include: Plasmodium falciparum and Toxoplasma gondii.

In another embodiment, the agent is an allergen. Allergic conditions include eczema, allergic rhinitis or coryza, hay fever, bronchial asthma, urticaria (hives) and food allergies, and other atopic conditions.

In another embodiment, the agent is a cancer or tumor. As used herein, a tumor or cancer includes, e.g., tumors of the brain, lung (e.g., small cell and non-small cell), ovary, breast, prostate, colon, as well as other carcinomas, melanomas, and sarcomas.

Recombinant gene expression vectors of the invention may be plasmids or cosmids, which include the antigen coding polynucleotides of the invention, but may also be viruses or retroviruses. The vectors used in the polynucleotide vaccines may be “naked” (i.e., not associated with a delivery vehicle such as liposomes, colloidal particles, etc.). For convenience, the term “plasmid” as used in this disclosure will refer to plasmids or cosmids, depending on which is appropriate to use for expression of the peptide of interest (where the choice between the two is dictated by the size of the gene encoding the peptide of interest). A commonly used plasmid vector which may be used is pBR322.

Various viral vectors that can be utilized in the invention include adenovirus, herpes virus, vaccinia, or an RNA virus such as a retrovirus. Retroviral vectors may be derivatives of a murine or avian retrovirus. Examples of retroviral include, but are not limited to: Moloney murine leukemia virus (MoMuLV), Harvey murine sarcoma virus (HaMuS-V), murine mammary tumor virus (MuMTV), and Rous Sarcoma Virus (RSV).

Other plasmids and viral vectors useful in the methods of the invention are well known in the vaccine art.

The recipient's thymus may be reactivated by disruption of sex steroid mediated signalling to the thymus. This disruption reverses the hormonal status of the recipient. In certain embodiments, the recipient is post-pubertal. According to the methods of the invention, the hormonal status of the recipient is reversed such that the hormones of the recipient approach pre-pubertal levels. By lowering the level of sex steroid hormones in the recipient, the signalling of these hormones to the thymus is lowered, thereby allowing the thymus to be reactivated.

A non-limiting method for creating disruption of sex steroid mediated signalling to the thymus is through castration. Methods for castration include, but are not limited to, chemical castration and surgical castration. During or after the castration step, hematopoietic stem or progenitor cells, or epithelial stem cells, from the donor are transplanted into the recipient. These cells are accepted by the thymus as belonging to the recipient and become part of the production of new T cells and DC by the thymus. The resulting population of T cells recognize both the recipient and donor as self, thereby creating tolerance for a graft from the donor.

One method of reactivating the thymus is by blocking the direct and/or indirect stimulatory effects of LHRH on the pituitary, which leads to a loss of the gonadotrophins FSH and LH. These gonadotrophins normally act on the gonads to release sex hormones, in particular estrogens in females and testosterone in males; the release is blocked by the loss of FSH and LH. The direct consequences of this are an immediate drop in the plasma levels of sex steroids, and as a result, progressive release of the inhibitory signals on the thymus. The degree and kinetics of thymic regrowth can be enhanced by injection of CD34⁺ hematopoietic cells (ideally autologous).

This invention may be used with any animal species (including humans) having sex steroid driven maturation and an immune system, such as mammals and marsupials. In some embodiments, the invention is used with large mammals, such as humans.

The terms “regeneration,” “reactivation” and “reconstitution” and their derivatives are used interchangeably herein, and refer to the recovery of an atrophied thymus to its active state. By “active state” is meant that a thymus in a patient whose sex steroid hormone mediated signalling to the thymus has been disrupted, achieves an output of T cells that is at least 10%, or at least 20%, or at least 40%, or at least 60%, or at least 80%, or at least 90% of the output of a pre-pubertal thymus (i.e., a thymus in a patient who has not reached puberty).

“Castration,” as used herein, means the elimination of sex steroid production and distribution in the body. This effectively returns the patient to pre-pubertal status when the thymus is fully functioning. Surgical castration removes the patient's gonads. Methods for surgically castration are well known to routinely trained veterinarians and physicians. One non-limiting method for castrating a male animal is described in the examples below. Other non-limiting methods for castrating human patients include a hysterectomy procedure (to castrate women) and surgical castration to remove the testes (to castrate men).

A less permanent version of castration is through the administration of a chemical for a period of time, referred to herein as “chemical castration.” A variety of chemicals are capable of functioning in this manner. Non-limiting examples of such chemicals are the sex steroid analogs described below. During the chemical delivery, and for a period of time afterwards, the patient's hormone production is turned off. The castration may be reversed upon termination of chemical delivery.

### Disruption of Sex Steroid Mediated Signalling to the Thymus

As will be readily understood, sex steroid mediated signaling to the thymus can be disrupted in a range of ways well known to those of skill in the art, some of which are described herein. For example, inhibition of sex steroid production or blocking of one or more sex steroid receptors within the thymus will accomplish the desired disruption, as will administration of sex steroid agonists and/or antagonists, or active (antigen) or passive (antibody) anti-sex steroid vaccinations.

Administration may be by any method which delivers the sex steroid ablating agent into the body. Thus, the sex steroid ablating agent maybe be administered, in accordance with the invention, by any route including, without limitation, intravenous, subdermal, subcutaneous, intramuscular, topical, and oral routes of administration. One non-limiting example of administration of a sex steroid ablating agent is a subcutaneous/intradermal injection of a “slow-release” depot of GnRH agonist (e.g., one, three, or four month Lupron® injections) or a subcutaneous/intradermal injection of a “slow-release” GnRH-containing implant (e.g., one or three month Zoladex®, e.g., 3.6 mg or 10.8 mg implant). These could also be given intramuscular (i.m.), intravenously (i.v.) or orally, depending on the appropriate formulation. Another example is by subcutaneous injection of a “depot” or “impregnated implant” containing, for example, about 30 mg of Lupron® (e.g., Lupron Depot®,(leuprolide acetate for depot suspension) TAP Pharmaceuticals Products, Inc., Lake Forest, Ill.). A 30 mg Lupron® injection is sufficient for four months of sex steroid ablation to allow the thymus to rejuvenate and export new naïve T cells into the blood stream.

In some embodiments, sex steroid ablation or inhibition of sex steroid signaling is accomplished by administering an anti-androgen such as an androgen blocker (e.g., bicalutamide, trade names Cosudex® or Casodex®, AstraZeneca, Aukland, NZ), either alone or in combination with an LHRH analog or any other method of castration. Sex steroid ablation or interruption of sex steroid signaling may also be accomplished by administering cyproterone acetate (trade name, Androcor®, Shering AG, Germany; e.g., 10-1000 mg, 100 mg bd or tds, or 300 mg IM weekly, a 17-hydroxyprogesterone acetate, which acts as a progestin, either alone or in combination with an LHRH analog or any other method of castration. Alternatively, other anti-androgens may be used (e.g., antifungal agents of the imidazole class, such as liarozole(Liazol® e.g., 150 mg/day, an aromatase inhibitor) and ketoconazole, bicalutamide (trade name Cosudex® or Casodex®, 5-500 mg, e.g., 50 mg po QID), flutamide (trade names Euflex® and Eulexin®, Shering Plough Corp, N.J.; 50-500 mg e.g., 250 or 750 po QID), megestrol acetate (Megace®) e.g., 480-840 mg/day or nilutamide (trade names Anandron®, and Nilandron®, Roussel, France e.g., orally, 150-300 mg/day)). Antiandrogens are often important in therapy, since they are commonly utilized to address flare by GnRH analogs. Some antiandrogens act by inhibiting androgen receptor translocation, which interrupts negative feedback resulting in increased testosterone levels and minimal loss of libido/potency. Another class of anti-androgens useful in the present invention are the selective androgen receptor modulators (SARMS) (e.g., quinoline derivatives, bicalutamide (trade name Cosudex® or Casodex®, ICI Pharmaceuticals, England e.g., orally, 50 mg/day), and flutamide (trade name Eulexin®, e.g., orally, 250 mg/day)). Other well known anti-androgens include 5 alpha reductase inhibitors (e.g., dutasteride,(e.g., 0.5 mg/day) which inhibits both 5 alpha reductase isoenzymes and results in greater and more rapid DHT suppression; finasteride (trade name Proscar®; 0.5-500 mg, e.g, 5 mg po daily), which inhibits 5 alpha reductase 2 and consequent DHT production, but has little or no effect on testosterone or LH levels);

In other embodiments, sex steroid ablation or inhibition of sex steroid signaling is accomplished by administering anti-estrogens either alone or in combination with an LHRH analog or any other method of castration. Some anti-estrogens (e.g., anastrozole (trade name Arimidex®), and fulvestrant (trade name Faslodex®) act by binding the estrogen receptor (ER) with high affinity similar to estradiol and consequently inhibiting estrogen from binding. Faslodex® binding also triggers conformational change to the receptor and down-regulation of estrogen receptors, without significant change in FSH or LH levels. Other non-limiting examples of anti-estrogens are tamoxifen (trade name Nolvadex®); Clomiphene (trade name Clomid®)e.g.,50-250mg/day, a non-steroidal ER ligand with mixed agonist/antagonist properties, which stimulates release of gonadotrophins; Fulvestrant (trade name Faslodex®; 10-1000 mg, e.g., 250 mg IM monthly); diethylstilbestrol ((DES), trade name Stilphostrol®) e.g.,1-3 mg/day, which shows estrogenic activity similar to, but greater than, that of estrone, and is therefore considered an estrogen agonist, but binds both androgen and estrogen receptors to induce feedback inhibition on FSH and LH production by the pituitary, diethylstilbestrol diphosphate e.g.,50 to 200 mg/day; as well as danazol, droloxifene, and iodoxyfene, which each act as antagonists. Another class of anti-estrogens which may be used either alone or in combination with other methods of castration, are the selective estrogen receptor modulators (SERMS) (e.g., toremifene (trade name Fareston®, 5-1000 mg, e.g., 60 mg po QID), raloxofene (trade name Evista®), and tamoxifen (trade name Nolvadex®, 1-1000 mg, e.g., 20 mg po bd), which behaves as an agonist at estrogen receptors in bone and the cardiovascular system, and as an antagonist at estrogen receptors in the mammary gland). Estrogen receptor downregulators (ERDs) (e.g., tamoxifen (trade name, Nolvadex®)) may also be used in the present invention.

Other non-limiting examples of methods of inhibiting sex steroid signalling which may be used either alone or in combination with other methods of castration, include aromatase inhibitors and other adrenal gland blockers (e.g., Aminoglutethimide, formestane, vorazole, exemestane, anastrozole (trade name Arimidex®, 0.1-100 mg, e.g., 1 mg po QID), which lowers estradiol and increases LH and testosterone), letrozole (trade name Femara®, 0.2-500 mg, e.g., 2.5 mg po QID), and exemestane (trade name Aromasin®)1-2000 mg, e.g., 25 mg/day); aldosterone antagonists (e.g., spironolactone (trade name, Aldactone®) e.g., 100 to 400 mg/day), which blocks the androgen cytochrome P-450 receptor;) and eplerenone, a selective aldosterone-receptor antagonist) antiprogestogens (e.g., medroxypregesterone acetate, e.g. 5 mg/day, which inhibits testosterone syntheses and LH synthesis); and progestins and anti-progestins such as the selective progesterone response modulators (SPRM) (e.g., megestrol acetate e.g.,160 mg/day, mifepristone (RU 486, Mifeprex®, e.g. 200 mg/day); and other compounds with estrogen/antiestrogenic activity, (e.g., phytoestrogens, flavones, isoflavones and coumestan derivatives, lignans, and industrial compounds with phenolic ring (e.g., DDT)). Also, anti-GnRH vaccines (see, e.g., Hsu et al., (2000) Cancer Res. 60:3701; Talwar, (1999) Immunol. Rev. 171:173-92), or any other pharmaceutical which mimics the effects produced by the aforementioned drugs, may also be used. In addition, steroid receptor based modulators, which may be targeted to be thymic specific, may also be developed and used. Many of these mechanisms of inhibiting sex steroid signaling are well known. Each drugs may also be used in modified form, such as acetates, citrates and other salts thereof, which are well known to those in the art.

Because of the complex and interwoven feedback mechanisms of the hormonal system, administration of sex steroids may result in inhibition of sex steroid signalling. For example, estradiol decreases gonadotropin production and sensitivity to GnRH action. However, higher levels of estradiol result in gonadotropin surge. Likewise, progesterone influences frequency and amount of LH release. In men, testosterone inhibits gonadotropin production. Estrogen administered to men decreases LH and testosterone, and anti-estrogen increases LH.

Inhibin A and B peptides made in the gonads in response to gonadotropins, down regulates the pituitary and suppress FSH. Activin normally up regulates GnRH receptors and stimulate FSH synthesis, however over production may shut down sex steroid production. Thus these hormones may also be the target of inhibition of sex steroid-mediated signalling.

In some embodiments, the sex steroid mediated signaling to the thymus is disrupted by administration of gonadotrophin-releasing hormone (GnRH) or an analog thereof. GnRH is a hypothalamic decapeptide that stimulates the secretion of the pituitary gonadotropins, leutinizing hormone (LH) and follicle-stimulating hormone (FSH). Thus, GnRH, e.g., in the form of Synarel or Lupron, will suppress the pituitary gland and stop the production of FSH and LH.

In some embodiments, the sex steroid mediated signaling to the thymus is disrupted by administration of a sex steroid analog, such as an analog of leutinizing hormone-releasing hormone (LHRH). Sex steroid analogs and their use in therapies and chemical castration are well known. Sex steroid analogs are commercially and their use in therapies and chemical castration are well known. Such analogs include, but are not limited to, the following agonists of the LHRH receptor (LHRH-R): buserelin (e.g., buserelin acetate, trade names Suprefact® (e.g., 0.5-02 mg s.c./day), Suprefact Depot®, and Suprefact® Nasal Spray (e.g., 2 μg per nostril, every 8 hrs.), Hoechst, also described in U.S. Pat. Nos. 4,003,884, 4,118,483, and 4,275,001); Cystorelin® (e.g., gonadorelin diacetate tetrahydrate, Hoechst); deslorelin (e.g., desorelin acetate, Deslorell®, Balance Pharmaceuticals); gonadorelin (e.g., gonadorelin hydrocholoride, trade name Factrel® (100 μg i.v. or s.c.), Ayerst Laboratories); goserelin (goserelin acetate, trade name Zoladex®, AstraZeneca, Aukland, NZ, also described in U.S. Pat. Nos. 4,100,274 and 4,128,638; GB 9112859 and GB 9112825); histrelin (e.g., histerelin acetate, Supprelin®, (s.c., 10 μg/kg.day), Ortho, also described in EP 217659); leuprolide (leuprolide acetate, trade name Lupron® or Lupron Depot®; Abbott/TAP, Lake Forest, Ill., also described in U.S. Pat. Nos. 4,490,291 3,972,859, 4,008,209, 4,992,421, and 4,005,063; DE 2509783); leuprorelin (e.g., leuproelin acetate, trade name Prostap SR® (e.g., single 3.75 mg dose s.c. or i.m./month), Prostap3® (e.g., single 11.25 mg dose s.c. every 3 months), Wyeth, USA, also described in Plosker et al., (1994) Drugs 48:930); lutrelin (Wyeth, USA, also described in U.S. Pat. No. 4,089,946); Meterelin® (e.g., Avorelina (e.g., 10-15 mg slow-release formulation), also described in EP 23904 and WO 91/18016); nafarelin (e.g., trade name Synarel® (i.n. 200-1800 μg/day), Syntex, also described in U.S. Pat. No. 4,234,571; WO 93/15722; and EP 52510); and triptorelin (e.g., triptorelin pamoate; trade names Trelstar LA® (11.25 mg over 3 months), Trelstar LA Debioclip® (pre-filled, single dose delivery), LA Trelstar Depot® (3.75 mg over one month), and Decapeptyl®, Debiopharm S. A., Switserland, also described in U.S. Pat. Nos. 4,010,125, 4,018,726, 4,024,121, and 5,258,492; EP 364819). LHRH analogs also include, but are not limited to, the following antagonists of the LHRH-R: abarelix (trade name Plenaxis™ (e.g., 100 mg i.m. on days 1, 15 and 29, then every 4 weeks thereafter), Praecis Pharmaceuticals, Inc., Cambridge, Mass.) and cetrorelix (e.g., cetrorelix acetate, trade name Cetrotide™ (e.g., 0.25 or 3 mg s.c.), Zentaris, Frankfurt, Germany). Additional sex steroid analogs include Eulexin® (e.g., flutamide (e.g., 2 capsules 2×/day, total 750 mg/day), Schering-Plough Corp., also described in FR 7923545, WO 86/01105 and PT 100899), and dioxane derivatives (e.g., those described in EP 413209), and other LHRH analogues such as are described in EP 181236, U.S. Pat. Nos. 4,608,251, 4,656,247, 4,642,332, 4,010,149, 3,992,365, and 4,010,149. Combinations of agonists, combinations of antagonists, and combinations of agonists and antagonists are also included. One non-limiting analog of the invention is deslorelin (described in U.S. Pat. No. 4,218,439). For a more extensive list, of analogs, see Vickery et al. (1984) LHRH and Its Analogs: Contraceptive & Therapeutic Applications (Vickery et al., eds.) MTP Press Ltd., Lancaster, Pa. Each analog may also be used in modified form, such as acetates, citrates and other salts thereof, which are well known to those in the art.

Doses of a sex steroid analog used, in according with the invention, to disrupt sex steroid hormone signaling to the thymus, can be readily determined by a routinely trained physician or veterinarian, and may be also be determined by consulting medical literature (e.g., The Physician's Desk Reference, 52nd edition, Medical Economics Company, 1998).

In certain embodiments, an LHRH-R antagonist is delivered to the patient, followed by an LHRH-R agonist. For example, the antagonist can be administered as a single injection of sufficient dose to cause castration within 5-8 days (this is normal for, e.g., Abarelix). When the sex steroids have reached this castrate level, the agonist is given. This protocol abolishes or limits any spike of sex steroid production, before the decrease in sex steroid production, that might be produced by the administration of the agonist. In an alternate embodiment, an LHRH-R agonist that creates little or no sex steroid production spike is used, with or without the prior administration of an LHRH-R antagonist.

Sex steroids comprise a large number of the androgen, estrogen and progestin family of hormone molecules. Non-limiting members of the progestin family of C21 steroids include progesterone, 17α-hydroxy progesterone, 20α-hydroxy progesterone, pregnanedione, pregnanediol and pregnenolone. Non-limiting members of the androgen family of C19 steroids include testosterone, androstenedione, dihydrotesterone (DHT), androstanedione, androstandiol, dehydroepiandrosterone and 17α-hydroxy androstenedione. Non-limiting members of the estrogen family of C17 steroids include estrone, estradiol-17α, and estradiol-17β.

Signalling by sex steroids is the net result of complex outcomes of the components of the pathway that includes biosynthesis, secretion, metabolism, compartmentalization and action. Parts of this pathway are not fully understood; nevertheless, there are numerous existing and potential mechanisms for achieving inhibition of sex steroid signalling. In one aspect of the present invention, inhibition of sex steroid signalling is achieved by modifying the bioavailable sex steroid hormone levels at the cellular level, the so called ‘free’ levels, by altering biosynthesis or metabolism, the binding to sex steroid receptors on or in target cells, and/or intracellular signalling of sex steroids.

Broadly speaking, it is possible to influence the signalling pathways either directly or indirectly. The direct methods would include methods of influencing sex steroid biosynthesis and metabolism, binding to the respective receptor and intracellular modification of the signal. The indirect methods would include those methods known to influence sex steroid hormone production and action such as the peptide hormone and growth factors present in the pituitary gland and the gonad. The latter would include but not be limited to follicle stimulating hormone (FSH), luteinizing hormone (LH) and activin made by the pituitary gland, and inhibin, activin and insulin-like growth factor-1 (IGF-1) made by the gonad.

The person skilled in the art will appreciate that inhibition of sex steroid signaling may take place by making the aforementioned modifications at the level of the relevant hormone, enzyme, receptor, binding molecule and/or ligand, either by direct action upon that molecule or by action upon a precursor of that molecule, including a nucleic acid that encodes or regulates it, or a molecule that can modify the action of sex steroid.

Direct Methods of Inhibiting Signalling

Biosynthesis

The rate of biosynthesis is the major rate determining step in the production of steroid hormones and hence the bioavailability of ‘free’ hormone in serum. Inhibition of a key enzyme such as P450 cholesterol side chain cleavage (P450 scc), early in the pathway, will reduce production of all the major sex steroids. On the other hand, inhibition of enzymes later in the pathway, such as P450 aromatase (P450 arom) that converts androgens to estrogens, or 5α-reductase that converts testosterone to DHT, will only effect the production of estrogens or DHT, respectively. Another important facet of sex steroid hormone biosynthesis is the family of oxidoreductase enzymes that catalyse the interconversion of inactive to bioactive steroids, for example, androstenedione to testosterone or estrone to estradiol-17β by 17-hydroxysteroid dehydrogenase (17-HSD). These enzymes are tissue and cell specific and generally catalyse either the reduction or oxidation reaction e.g., 17P HSD type 3 is found exclusively in the Leydig cells of the testes, whereas 17β HSD type 1 is found in the ovary. They therefore offer the possibility of specifically reducing production of the active forms of androgens or estrogens.

There are many known inhibitors of the enzymes in the steroid biosynthesis pathway that are either already in clinical use or are under development. Some examples of these together with their treatment modalities are listed below. It is important that the action of these enzyme inhibitors does not unduly influence production of other steroids such as glucocorticoids and mineralocorticoids from the adrenal gland that are essential for metabolic stability. When using such inhibitors, it may be necessary to provide the patient with replacement glucocorticoids and sometimes mineralocorticoids.

Sex steroid biosynthesis occurs in varied sites and utilizing multiple pathways, predominantly produced the ovaries and testes, but there is some production in the adrenals, as well as synthesis of derivatives in other tissues, such as fat. Thus multiple mechanisms of inhibiting sex steroid signaling may be required to ensure adequate inhibition to achieve the present invention.

Metabolism and Compartmentalization

Sex steroid hormones have a short half-life in blood, generally only several minutes, due to the rapid metabolism, particularly by the liver, and clearance by the kidney and fat. Metabolism includes conjugation by glycosylation and sulphation, as well as reduction. Some of these metabolites retain biological activity either as prohormones, for example estrone sulphate, or through intrinsic bioactivity such as the reduced androgens. Any interference in the rate of metabolism can influence the ‘free’ levels of sex steroid hormones., however methods of achieving this are not currently available as are methods of influencing biosynthesis.

Another method of reducing the level of ‘free’ sex steroid hormone is by compartmentalization by binding of the sex steroid hormone to proteins present in the serum such as sex hormone binding globulin, corticosteroid-binding globulin, albumin and testosterone-estradiol binding globulin. Binding to sex steroid ligands, such as carrier molecules may make sex steroids unavailable for receptor binding. Increased binding may result from increased levels of carriers, such as SHBG or introduction of other ligands which bind the sex steroids, such as soluble receptors. Alternatively decreased levels of carrier molecules may make sex steroids more susceptible to degradation.

Active or passive immunization against a particular sex steroid hormone is a form of compartmentalization. There are examples in the literature of this approach successfully increasing ovulation rates in animals after immunization against estrogen or androgen. Sex steroids are secreted from cells in secretory vesicles. Inhibition or modification of the secretory mechanism is another method of inhibiting sex steroid signaling

Receptors & Intracellular Signalling

The sex steroids act on cells via specific receptors that can be either intracellular, or, as shown more recently, on the target cell membrane.

The intracellular receptors are members of the nuclear receptor superfamily. They are located in the cytoplasm of the cell and are transported to the nucleus after binding with the sex steroid hormone where they alter the transcription of specific genes. Receptors for the sex steroid hormones exist in several forms. Well known in the literature are two forms of the progesterone receptor, PRA and PRB, and three forms of the estrogen receptor, ERα, ERβ1 and ERβ2. Transcription of genes in response to the binding of the sex steroid hormone receptor to the steroid response element in the promoter region of the gene can be modified in a number of ways. Co-activators and co-repressors exist within the nucleus of the target cell that can modify binding of the steroid-receptor complex to the DNA and thereby effect transcription. The identity of many of these co-activators and co-repressors are known and methods of modifying their actions on steroid receptors are the topic of current research. Examples of the transcription factors involved in sex steroid hormone action are NF-1, SP1, Oct-1 and TFIID. These co-regulators are required for the full action of the steroids. Methods of modifying the actions of these nuclear regulators could involve the balance between activator and repressor by the use of antagonists or through control of expression of the genes encoding the regulators.

More recently, specific receptors for estrogens and progesterone have been identified on the membranes of cells whose structures are different from the intracellular PR. Unlike the classical steroid receptors that act on the genome, these receptors deliver a rapid non-genomic action via intracellular pathways that are not yet fully understood. One report suggests that estrogens interacting with membrane receptors activate the sphingosine pathway that is related to cell proliferation.

There are methods available or in development to alter the action of steroids via their cytoplasmic receptors. In this case, antiandrogens, antiestrogens and antiprogestins that interact with the specific steroid receptors, are well known in the literature and are in clinical use, as described below. Their action may be to compete for, or block the receptor, to modify receptor levels, sensitivity, conformation, associations or signaling. These drugs come in a variety of forms, steroidal and non-steroidal, competitive and non-competitive. Of particular interest are the selective receptor modulators, SARMS, SERMS and SPRM, which are targeted to particular tissues and are exemplified below.

Down regulation of receptors can be achieved in 2 ways; first, by excess agonist (steroid ligand), and second, by inhibiting transcription of the respective gene that encodes the receptor. The first method can be achieved through the use of selective agonists such as tamoxifen. The second method is not yet in clinical use.

Indirect Methods of Inhibiting Signalling

Biosynthesis

One of the indirect methods of inhibiting sex steroid signalling involves down regulation of the biosynthesis of the respective steroid by a modification to the availability or action of the pituitary gonadotrophins, FSH and LH, that are responsible for driving the biosynthesis of the sex steroid hormones in the gonad. One established inhibitor of FSH secretion is inhibin, a hormone produced by the gonads in response to FSH. Administration of inhibin to animals has been shown to reduce FSH levels in serum due to a decrease in the pituitary secretion of FSH. The best known way of accomplishing a reduction in both gonadotrophins is via the hypothalamic hormone, Gonadotrophin Releasing Hormone (GnRH), also known as Luteinizing Hormone Releasing Hormone (LHRH), which drives the pituitary synthesis and secretion of FSH and LH. Agonists and antagonists of GnRH that reduce the secretion of FSH and LH, and hence gonadal sex steroid production, are now available for clinical use, as described below.

Another indirect method of reducing the biosynthesis of sex steroid hormones is to modify the action of FSH and LH at the level of the gonad. This could be achieved by using antibodies directed against FSH and LH, or molecules designed to compete with FSH and LH for their respective receptors on gonadal cells that produce the sex steroid hormones. Another method of modifying the action of FSH and LH on gonadal cells is by a co-regulator of gonadotrophin action. For example, activin can reduce the capacity of the theca cells of the ovary and the Leydig cells of the testis to produce androgen in response to LH.

Modification may take place at the level of hormone precursors such as inhibition of cleavage of a signal peptide, for example the signal peptide of GnRH.

Receptors & Intracellular Signalling

Indirect methods of altering the signalling action of the sex steroid hormones include down regulation of the receptor pathways leading to the genomic or non-genomic actions of the steroids. An example of this is the capacity of progesterone to down regulate the level of ER in target tissues. Future methods will include treatment with molecules known to influence the co-regulators of the receptors in the cell nucleus leading to a decrease in the capacity of the cell to respond to the steroid.

While the stimulus for thymic reactivation is fundamentally based on the inhibition of the effects of sex steroids and/or the direct effects of the LHRH analogs, it may be useful to include additional substances which can act in concert to enhance the thymic effect. Such compounds include but are not limited to Interleukin 2 (IL-2), Interleukin 7 (IL-7), Interleukin 15 (IL-15), members of the epithelial and fibroblast growth factor families, stem cell factor (SCF), granulocyte colony stimulating factor (GCSF) and keratinocyte growth factor (KGF) (see, e.g., Sempowski et al., 2000; Andrew and Aspinall, 2001; Rossi et al., 2002). It is envisaged that these additional compound(s) would only be given once at the initial LHRH analog application. Each of these could be given in combination with the agonist, antagonist or any other form of sex steroid disruption. Since the growth factors have a relatively rapid half-life (e.g., in the hours) they may need to be given each day (e.g., every day for 7 days). The growth factors/cytokines would be given in the optimal form to preserve their biological activities, as prescribed by the manufacturer. Most likely this would be as purified proteins. However, additional doses of any one or combination of these substances may be given at any time to further stimulate the thymus. In addition, steroid receptor based modulators, which may be targeted to be thymic specific, may be developed and used.

As will be understood by persons skilled in the art at least some of the means for disrupting sex steroid signalling to the thymus will only be effective as long as the appropriate compound is administered. As a result, an advantage of certain embodiments of the present invention is that once the desired immunological affects of the present invention have been achieved, (2-3 months) the treatment can be stopped and thee subjects reproductive system will return to normal.

### Pharmaceutical Compositions

The compounds used in this invention can be supplied in any pharmaceutically acceptable carrier or without a carrier. Formulations of pharmaceutical compositions can be prepared according to standard methods (see, e.g., Remington, The Science and Practice of Pharmacy, Gennaro A. R., ed., 20^{th} edition, Williams & Wilkins Pa., USA 2000). Non-limiting examples of pharmaceutically acceptable carriers include physiologically compatible coatings, solvents and diluents. For parenteral, subcutaneous, intravenous and intramuscular administration, the compositions may be protected such as by encapsulation. Alternatively, the compositions may be provided with carriers that protect the active ingredient(s), while allowing a slow release of those ingredients. Numerous polymers and copolymers are known in the art for preparing time-release preparations, such as various versions of lactic acid/glycolic acid copolymers. See, for example, U.S. Pat. No. 5,410,016, which uses modified polymers of polyethylene glycol (PEG) as a biodegradable coating.

Formulations intended to be delivered orally can be prepared as liquids, capsules, tablets, and the like. These compositions can include, for example, excipients, diluents, and/or coverings that protect the active ingredient(s) from decomposition. Such formulations are well known (see, e.g., Remington, The Science and Practice of Pharmacy, Gennaro A. R., ed., 20^{th} edition, Williams & Wilkins Pa., USA 2000).

In any of the formulations of the invention, other compounds that do not negatively affect the activity of the LHRH analogs (i.e., compounds that do not block the ability of an LHRH analog to disrupt sex steroid hormone signalling to the thymus) may be included. Examples are various growth factors and other cytokines as described herein.

### Dose

The LHRH analog can be administered in a one-time dose that will last for a period of time. In certain embodiments, the formulation will be effective for one to two months. The standard dose varies with type of analog used. In general, the dose is between about 0.01 μg/kg and about 10 mg/kg, or between about 0.01 mg/kg and about 5 mg/kg. Dose varies with the LHRH analog or vaccine used. In certain embodiments, a dose is prepared to last as long as a periodic epidemic lasts. For example, “flu season” occurs usually during the winter months. A formulation of an LHRH analog can be made and delivered as described herein to protect a patient for a period of two or more months starting at the beginning of the flu season, with additional doses delivered every two or more months until the risk of infection decreases or disappears.

Genetic Modification of Haemopoietic Stem Cells (HSC)

Methods for isolating and transducing stems cells and progenitor cells would be well known to those skilled in the art. Examples of these types of processes are described, for example, in PCT Publication No. WO 95/08105, U.S. Pat. No. 5,559,703, U.S. Patent No. 5,399,493, U.S. Pat. No. 5,061,620, PCT Publication No. WO 96/33281, PCT Publication No. WO 96/33282, U.S. Pat. No. 5,681,559 and U.S. Pat. No. 5,199,942.

Antisense Polynucleotides

The term “antisense”, as used herein, refers to polynucleotide sequences which are complementary to a polynucleotide of the present invention. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Catalytic Nucleic Acids

The term catalytic nucleic acid refers to a DNA molecule or DNA containing molecule (also known in the art as a “deoxyribozyme” or “DNAzyme” ) or an RNA or RNA-containing molecule (also known as a “ribozyme” ) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity. The catalytic strand cleaves a specific site in a target nucleic acid. The types of ribozymes that are particularly useful in this invention are the hammerhead ribozyme (Haseloff and Gerlach 1988, Perriman et al., 1992) and the hairpin ribozyme (Shippy et al., 1999).

dsRNA

dsRNA is particularly useful for specifically inhibiting the production of a particular protein. Although not wishing to be limited by theory, Dougherty and Parks (1995) have provided a model for the mechanism by which dsRNA can be used to reduce protein production. This model has recently been modified and expanded by Waterhouse et al. (1998). This technology relies on the presence of dsRNA molecules that contain a sequence that is essentially identical to the mRNA of the gene of interest, in this case an mRNA encoding a polypeptide according to the first aspect of the invention. Conveniently, the dsRNA can be produced in a single open reading frame in a recombinant vector or host cell, where the sense and antisense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Dougherty and Parks (1995), Waterhouse et al. (1998), and PCT Publication Nos. WO 99/32619, WO 99/53050, WO 99/49029, and WO 01/34815.

Anti-HIV Constructs

Those skilled in the art would be able to develop suitable anti-HIV constructs for use in the present invention. Indeed, a number of anti-HIV antisense constructs and ribozymes have already been developed and are described, for example; in U.S. Pat. No. 5,811,275, U.S. Pat. No. 5,741,706, PCT Publication No. WO 94/26877, Australian Patent Application No. 56394/94 and U.S. Pat. No. 5,144,019.

### Delivery of Agents for Chemical Castration

Delivery of the compounds of this invention can be accomplished via a number of methods known to persons skilled in the art. One standard procedure for administering chemical inhibitors to inhibit sex steroid mediated signalling to the thymus utilizes a single dose of an LHRH agonist that is effective for three months. For this a simple one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half-life of the inhibitor within the body, such as by modification of the chemical, while retaining the function required herein, may be used.

Examples of more useful delivery mechanisms include, but are not limited to, laser irradiation of the skin, and creation of high pressure impulse transients (also called stress waves or impulse transients) on the skin, each method accompanied or followed by placement of the compound(s) with or without carrier at the same locus. One method of this placement is in a patch placed and maintained on the skin for the duration of the treatment.

One means of delivery utilizes a laser beam, specifically focused, and lasing at an appropriate wavelength, to create small perforations or alterations in the skin of a patient. See U.S. Pat. No. 4,775,361, U.S. Pat. No. 5,643,252, U.S. Pat. No. 5,839,446, U.S. Pat. No. 6,056,738, U.S. Pat. No. 6,315,772, and U.S. Pat. No. 6,251,099, all of which are incorporated herein by reference. In one embodiment, the laser beam has a wavelength between 0.2 and 10 microns. The wavelength may be between about 1.5 and 3.0 microns. In one embodiment, the wavelength is about 2.94 microns. In one embodiment, the laser beam is focused with a lens to produce an irradiation spot on the skin through the epidermis of the skin. In an additional embodiment, the laser beam is focused to create an irradiation spot only through the stratum corneum of the skin.

As used herein, “ablation” and “perforation” mean a hole created in the skin. Such a hole can vary in depth; for example it may only penetrate the stratum corneum, it may penetrate all the way into the capillary layer of the skin, or it may terminate anywhere in between. As used herein, “alteration” means a change in the skin structure, without the creation of a hole, that increases the permeability of the skin. As with perforation, skin can be altered to any depth.

Several factors may be considered in defining the laser beam, including wavelength, energy fluence, pulse temporal width and irradiation spot-size. In one embodiment, the energy fluence is in the range of 0.03-100,000 J/cm2. The energy fluence may be in the range of 0.03-9.6 J/cm². The beam wavelength is dependent in part on the laser material, such as Er:YAG. The pulse temporal width is a consequence of the pulse width produced by, for example, a bank of capacitors, the flashlamp, and the laser rod material. The pulse width is optimally between 1 fs (femtosecond) and 1,000 μs.

According to this method the perforation or alteration produced by the laser need not be produced with a single pulse from the laser. In one embodiment a perforation or alteration through the stratum corneum is produced by using multiple laser pulses, each of which perforates or alters only a fraction of the target tissue thickness.

To this end, one can roughly estimate the energy required to perforate or alter the stratum corneum with multiple pulses by taking the energy in a single pulse and dividing by the number of pulses desirable. For example, if a spot of a particular size requires 1 J of energy to produce a perforation or alteration through the entire stratum corneum, then one can produce qualitatively similar perforation or alteration using ten pulses, each having 1/10th the energy. Because it is desirable that the patient not move the target tissue during the irradiation (human reaction times are on the order of 100 ms or so), and that the heat produced during each pulse not significantly diffuse, in one embodiment the pulse repetition rate from the laser should be such that complete perforation is produced in a time of less than 100 ms. Alternatively, the orientation of the target tissue and the laser can be mechanically fixed so that changes in the target location do not occur during the longer irradiation time.

To penetrate the skin in a manner that induces little or no blood flow, skin can be perforated or altered through the outer surface, such as the stratum corneum layer, but not as deep as the capillary layer. The laser beam is focused precisely on the skin, creating a beam diameter at the skin in the range of approximately 0.5 microns −5.0 cm. Optionally, the spot can be slit-shaped, with a width of about 0.05-0.5 mm and a length of up to 2.5 mm. The width can be of any size, being controlled by the anatomy of the area irradiated and the desired permeation rate of the fluid to be removed or the pharmaceutical applied. The focal length of the focusing lens can be of any length, but in one embodiment it is 30 mm.

By modifying wavelength, pulse length, energy fluence (which is a function of the laser energy output (in Joules) and size of the beam at the focal point (cm2)), and irradiation spot size, it is possible to vary the effect on the stratum corneum between ablation (perforation) and non-ablative modification (alteration). Both ablation and non-ablative alteration of the stratum corneum result in enhanced permeation of subsequently applied pharmaceuticals.

For example, by reducing the pulse energy while holding other variables constant, it is possible to change between ablative and non-ablative tissue-effect. Using an Er:YAG laser having a pulse length of about 300 μs, with a single pulse or radiant energy and irradiating a 2 mm spot on the skin, a pulse energy above approximately 100 mJ causes partial or complete ablation, while any pulse energy below approximately 100 mJ causes partial ablation or non-ablative alteration to the stratum corneum. Optionally, by using multiple pulses, the threshold pulse energy required to enhance permeation of body fluids or for pharmaceutical delivery is reduced by a factor approximately equal to the number of pulses.

Alternatively, by reducing the spot size while holding other variables constant, it is also possible to change between ablative and non-ablative tissue-effect. For example, halving the spot area will result in halving the energy required to produce the same effect. Irradiation down to 0.5 microns can be obtained, for example, by coupling the radiant output of the laser into the objective lens of a microscope objective (e.g., as available from Nikon, Inc., Melville, N.Y.). In such a case, it is possible to focus the beam down to spots on the order of the limit of resolution of the microscope, which is perhaps on the order of about 0.5 microns. In fact, if the beam profile is Gaussian, the size of the affected irradiated area can be less than the measured beam size and can exceed the imaging resolution of the microscope. To non-ablatively alter tissue in this case, it would be suitable to use a 3.2 J/cm² energy fluence, which for a half-micron spot size would require a pulse energy of about 5 nJ. This low a pulse energy is readily available from diode lasers, and can also be obtained from, for example, the Er:YAG laser by attenuating the beam by an absorbing filter, such as glass.

Optionally, by changing the wavelength of radiant energy while holding the other variables constant, it is possible to change between an ablative and non-ablative tissue-effect. For example, using Ho:YAG (holmium: YAG; 2.127 microns) in place of the Er:YAG (erbium: YAG; 2.94 microns) laser, would result in less absorption of energy by the tissue, creating less of a perforation or alteration.

Picosecond and femtosecond pulses produced by lasers can also be used to produce alteration or ablation in skin. This can be accomplished with modulated diode or related microchip lasers, which deliver single pulses with temporal widths in the 1 femtosecond to 1 ms range. (See D. Stern et al., “Corneal Ablation by Nanosecond, Picosecond, and Femtosecond Lasers at 532 and 625 nm,” Corneal Laser Ablation, Vol. 107, pp. 587-592 (1989), incorporated herein by reference, which discloses the use of pulse lengths down to 1 femtosecond).

Another delivery method uses high pressure impulse transients on skin to create permeability. See U.S. Pat. No. 5,614,502, and U.S. Pat. No. 5,658,892, both of which are incorporated herein by reference. High pressure impulse transients, e.g., stress waves (e.g., laser stress waves (LSW) when generated by a laser), with specific rise times and peak stresses (or pressures), can safely and efficiently effect the transport of compounds, such as those of the present disclosure, through layers of epithelial tissues, such as the stratum corneum and mucosal membranes. These methods can be used to deliver compounds of a wide range of sizes regardless of their net charge. In addition, impulse transients used in the present methods avoid tissue injury.

Prior to exposure to an impulse transient, an epithelial tissue layer, e.g., the stratum corneum, is likely impermeable to a foreign compound; this prevents diffusion of the compound into cells underlying the epithelial layer. Exposure of the epithelial layer to the impulse transients enables the compound to diffuse through the epithelial layer. The rate of diffusion, in general, is dictated by the nature of the impulse transients and the size of the compound to be delivered.

The rate of penetration through specific epithelial tissue layers, such as the stratum corneum of the skin, also depends on several other factors including pH, the metabolism of the cutaneous substrate tissue, pressure differences between the region external to the stratum corneum, and the region internal to the stratum corneum, as well as the anatomical site and physical condition of the skin. In turn, the physical condition of the skin depends on health, age, sex, race, skin care, and history. For example, prior contacts with organic solvents or surfactants affect the physical condition of the skin.

The amount of compound delivered through the epithelial tissue layer will also depend on the length of time the epithelial layer remains permeable, and the size of the surface area of the epithelial layer which is made permeable.

The properties and characteristics of impulse transients are controlled by the energy source used to create them. See WO 98/23325, which is incorporated herein by reference. However, their characteristics are modified by the linear and non-linear properties of the coupling medium through which they propagate. The linear attenuation caused by the coupling medium attenuates predominantly the high frequency components of the impulse transients. This causes the bandwidth to decrease with a corresponding increase in the rise time of the impulse transient. The non-linear properties of the coupling medium, on the other hand, cause the rise time to decrease. The decrease of the rise time is the result of the dependence of the sound and particle velocity on stress (pressure). As the stress increases, the sound and the particle velocity increase as well. This causes the leading edge of the impulse transient to become steeper. The relative strengths of the linear attenuation, non-linear coefficient, and the peak stress determine how long the wave has to travel for the increase in steepness of rise time to become substantial.

The rise time, magnitude, and duration of the impulse transient are chosen to create a non-destructive (i.e., non-shock wave) impulse transient that temporarily increases the permeability of the epithelial tissue layer. Generally the rise time is at least 1 ns, and may be about 10 ns.

The peak stress or pressure of the impulse transients varies for different epithelial tissue or cell layers. For example, to transport compounds through the stratum corneum, the peak stress or pressure of the impulse transient should be set to at least 400 bar; at least 1,000 bar, but no more than about 2,000 bar. For epithelial mucosal layers, the peak pressure should be set to between 300 bar and 800 bar, and may be between 300 bar and 600 bar. The impulse transients may have durations on the order of a few tens of ns, and thus interact with the epithelial tissue for only a short period of time. Following interaction with the impulse transient, the epithelial tissue is not permanently damaged, but remains permeable for up to about three minutes.

In addition, these methods involve the application of only a few discrete high amplitude pulses to the patient. The number of impulse transients administered to the patient is typically less than 100, less than 50, or may be less than 10. When multiple optical pulses are used to generate the impulse transient, the time duration between sequential pulses is 10 to 120 seconds, which is long enough to prevent permanent damage to the epithelial tissue.

Properties of impulse transients can be measured using methods standard in the art. For example, peak stress or pressure, and rise time can be measured using a polyvinylidene fluoride (PVDF) transducer method as described in Doukas et al., Ultrasound Med. Biol. 21:961 (1995).

Impulse transients can be generated by various energy sources. The physical phenomenon responsible for launching the impulse transient is, in general, chosen from three different mechanisms: (1) thermoelastic generation; (2) optical breakdown; or (3) ablation.

For example, the impulse transients can be initiated by applying a high energy laser source to ablate a target material, and the impulse transient is then coupled to an epithelial tissue or cell layer by a coupling medium. The coupling medium can be, for example, a liquid or a gel, as long as it is non-linear. Thus, water, oil such as castor oil, an isotonic medium such as phosphate buffered saline (PBS), or a gel such as a collagenous gel, can be used as the coupling medium.

In addition, the coupling medium can include a surfactant that enhances transport, e.g., by prolonging the period of time in which the stratum corneum remains permeable to the compound following the generation of an impulse transient. The surfactant can be, e.g., ionic detergents or nonionic detergents and thus can include, e.g., sodium lauryl sulfate, cetyl trimethyl ammonium bromide, and lauryl dimethyl amine oxide.

The absorbing target material acts as an optically triggered transducer. Following absorption of light, the target material undergoes rapid thermal expansion, or is ablated, to launch an impulse transient. Typically, metal and polymer films have high absorption coefficients in the visible and ultraviolet spectral regions.

Many types of materials can be used as the target material in conjunction with a laser beam, provided they fully absorb light at the wavelength of the laser used. The target material can be composed of a metal such as aluminum or copper; a plastic, such as polystyrene, e.g., black polystyrene; a ceramic; or a highly concentrated dye solution. The target material must have dimensions larger than the cross-sectional area of the applied laser energy. In addition, the target material must be thicker than the optical penetration depth so that no light strikes the surface of the skin. The target material must also be sufficiently thick to provide mechanical support. When the target material is made of a metal, the typical thickness will be 1/32 to 1/16 inch. For plastic target materials, the thickness will be 1/16 to ⅛ inch.

Impulse transients can also be enhanced using confined ablation. In confined ablation, a laser beam transparent material, such as a quartz optical window, is placed in close contact with the target material. Confinement of the plasma, created by ablating the target material by using the transparent material, increases the coupling coefficient by an order of magnitude (Fabro et al., J. Appl. Phys. 68:775, 1990). The transparent material can be quartz, glass, or transparent plastic.

Since voids between the target material and the confining transparent material allow the plasma to expand, and thus decrease the momentum imparted to the target, the transparent material may be bonded to the target material using an initially liquid adhesive, such as carbon-containing epoxies, to prevent such voids.

The laser beam can be generated by standard optical modulation techniques known in the art, such as by employing Q-switched or mode-locked lasers using, for example, electro- or acousto-optic devices. Standard commercially available lasers that can operate in a pulsed mode in the infrared, visible, and/or infrared spectrum include Nd:YAG, Nd:YLF, CO₂, excimer, dye, Ti:sapphire, diode, holmium (and other rare-earth materials), and metal-vapor lasers. The pulse widths of these light sources are adjustable, and can vary from several tens of picoseconds (ps) to several hundred microseconds. For use in the present disclosure, the optical pulse width can vary from 100 ps to about 200 ns and may be between about 500 ps and 40 ns.

Impulse transients can also be generated by extracorporeal lithotripters (one example is described in Coleman et al., Ultrasound Med. Biol. 15:213-227, 1989). These impulse transients have rise times of 30 to 450 ns, which is longer than laser-generated impulse transients. To form an impulse transient of the appropriate rise time for the new methods using an extracorporeal lithotripter, the impulse transient is propagated in a non-linear coupling medium (e.g., water) for a distance determined by equation (1), above. For example, when using a lithotripter creating an impulse transient having a rise time of 100 ns and a peak pressure of 500 barr, the distance that the impulse transient should travel through the coupling medium before contacting an epithelial cell layer is approximately 5 mm.

An additional advantage of this approach for shaping impulse transients generated by lithotripters is that the tensile component of the wave will be broadened and attenuated as a result of propagating through the non-linear coupling medium. This propagation distance should be adjusted to produce an impulse transient having a tensile component that has a pressure of only about 5 to 10% of the peak pressure of the compressive component of the wave. Thus, the shaped impulse transient will not damage tissue.

The type of lithotripter used is not critical. Either an electrohydraulic, electromagnetic, or piezoelectric lithotripter can be used.

The impulse transients can also be generated using transducers, such as piezoelectric transducers. The transducer may be in direct contact with the coupling medium, and undergoes rapid displacement following application of an optical, thermal, or electric field to generate the impulse transient. For example, dielectric breakdown can be used, and is typically induced by a high-voltage spark or piezoelectric transducer (similar to those used in certain extracorporeal lithotripters, Coleman et al., Ultrasound Med. Biol. 15:213-227, 1989). In the case of a piezoelectric transducer, the transducer undergoes rapid expansion following application of an electrical field to cause a rapid displacement in the coupling medium.

In addition, impulse transients can be generated with the aid of fiber optics. Fiber optic delivery systems are particularly maneuverable and can be used to irradiate target materials located adjacent to epithelial tissue layers to generate impulse transients in hard-to reach places. These types of delivery systems, when optically coupled to lasers, may be used as they can be integrated into catheters and related flexible devices, and used to irradiate most organs in the human body. In addition, to launch an impulse transient having the desired rise times and peak stress, the wavelength of the optical source can be easily tailored to generate the appropriate absorption in a particular target material.

Alternatively, an energetic material can produce an impulse transient in response to a detonating impulse. The detonator can detonate the energetic material by causing an electrical discharge or spark.

Hydrostatic pressure can be used in conjunction with impulse transients to enhance the transport of a compound through the epithelial tissue layer. Since the effects induced by the impulse transients last for several minutes, the transport rate of a drug diffusing passively through the epithelial cell layer along its concentration gradient can be increased by applying hydrostatic pressure on the surface of the epithelial tissue layer, e.g., the stratum corneum of the skin, following application of the impulse transient.

### Improvement of Vaccine Response

By the methods described herein, the sex steroid-induced atrophic thymus is dramatically restored structurally and functionally to approximately its optimal pre-pubertal capacity in all currently definable terms. This includes the number, type and proportion of all T cell subsets. Also included are the complex stromal cells and their three dimensional architecture which constitute the thymic microenvironment required for producing T cells. The newly generated T cells emigrate from the thymus and restore peripheral T cell levels and function.

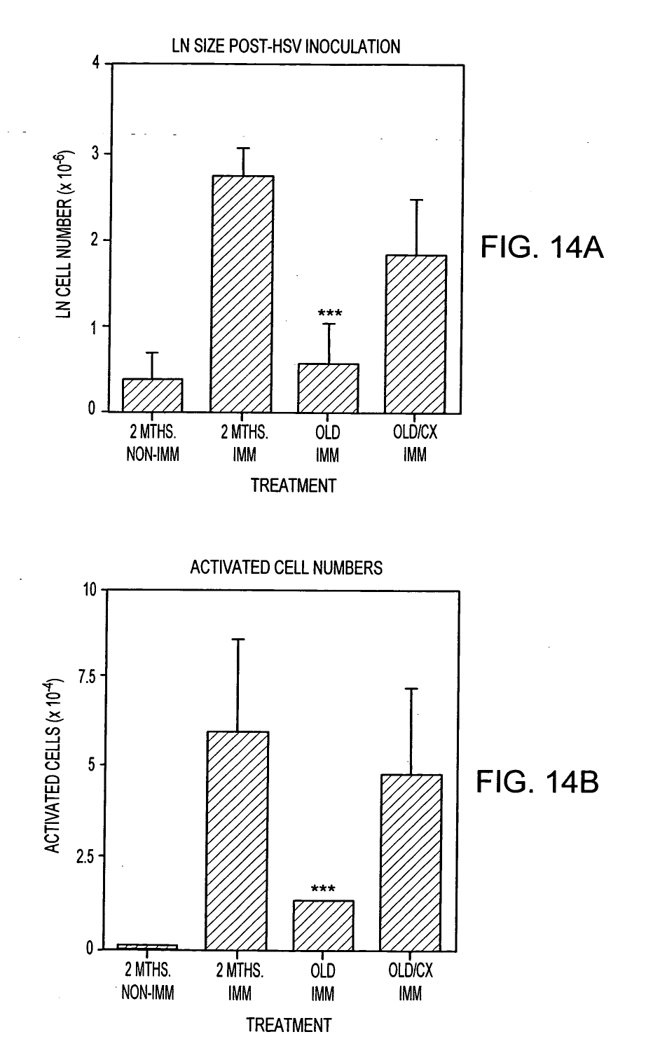

At this stage, the patient's immune system is rejuvenated and reactivated, thereby increasing its response to foreign antigens such as viruses and bacteria. This is shown, for example, in FIGS. 14-19, which show the effects of thymic reactivation on the mouse immune system, as demonstrated with viral (HSV) challenge. The mice having prior reactivation of the thymus demonstrate resistance to HSV infection, while those not having thymic reactivation (aged thymus) have higher levels of HSV infection. It is well known that the mouse immune system is very similar to the human immune system, and results in mice can be projected to show human responses. This is reinforced by the data showing the effects of thymic reactivation in humans.

The reactivation of the thymus can be supplemented by the addition of CD34⁺ hematopoietic stem cells (HSC) and/or epithelial stem cells slightly before or at the time the thymus begins to regenerate. Ideally these cells are autologous or syngeneic and have been obtained from the patient or twin prior to thymus reactivation. The HSC can be obtained by sorting CD34⁺ cells from the patient's blood and/or bone marrow. The number of HSC can be enhanced in several ways, including (but not limited to) by administering G-CSF (Neupogen, Amgen) to the patient prior to collecting cells, culturing the collected cells in Stem Cell Growth Factor, and/or administering G-CSF to the patient after CD34⁺ cell supplementation. Alternatively, the CD34⁺ cells need not be sorted from the blood or BM if their population is enhanced by prior injection of G-CSF into the patient.

In one embodiment, hematopoietic cells are supplied to the patient during thymic reactivation, which increases the immune capabilities of the patient's body.

The HSC are administered to the patient and migrate through the peripheral blood system to the thymus. The uptake into the thymus of these hematopoietic precursor cells is substantially increased in the absence of sex steroids. These cells become integrated into the thymus and produce dendritic cells and T cells. The results are a population of T cells and other immune cells that circulate in the peripheral blood of the recipient, and the accompanying increase in the population of cells, tissues and organs caused by reactivation of the patient's thymus, which are capable improved responses to the vaccine antigen.

Within 3-4 weeks of the start of blockage of sex steroid mediated signaling (approximately 2-3 weeks after the initiation of LHRH treatment), the first new T cells are present in the blood stream. Full development of the T cell pool, however, may take 3-4 months. Vaccination may begin soon after the appearance of the newly produced naïve cells; however, the wait may be 4-6 weeks after the initiation of LHRH therapy to begin vaccination, when enough new T cells to create a strong response will have been produced and will have undergone any necessary post-thymic maturation

This procedure can be combined with any other form of immune system stimulation, including adjuvant, accessory molecules, and cytokine therapies. For example, useful cytokines include but are not limited to interleukin 2 (IL-2) as a general immune growth factor, IL-4 to skew the response to Th2 (humoral immunity), and IFNγ to skew the response to Th1 (cell mediated, inflammatory responses). Accessory molecules include but are not limited to inhibitors of CTLA4, which enhance the general immune response by facilitating the CD28/B7.1,B7.2 stimulation pathway, which is normally inhibited by CTLA4.

### Effects on the Bone Marrow and HSC

The present disclosure also provides methods for increasing the production of bone marrow in a patient, including increasing production of HSC. This is useful in a number of applications. For example, one of the difficult side effects of chemotherapy, whether given for cancer or for another purpose, can be its negative impact on the patient's bone marrow. Depending on the dose of chemotherapy, the bone marrow may be ablated and production of blood cells may be impeded. Administration of a dose of LHRH analog according to this invention after chemotherapy treatment helps to reverse the damage done by the chemotherapy to the bone marrow and blood cells. Alternatively, administration of the LHRH analog in the weeks prior to delivery of chemotherapy will increase the population of HSC and other blood cells so that the impact of chemotherapy will be decreased.

In some chemotherapy regimens, such as high dose chemotherapy to treat any of the blood cancers, ablation of the bone marrow is a desired effect. The methods of this invention may be used immediately after ablation occurs to stimulate the bone marrow and increase the production of HSC and their progeny blood cells, so as to decrease the patient's recovery time. Following administration of the chemotherapy, usually allowing one or more days for the chemotherapy to clear from the patient's body, a dose of LHRH analog according to the methods described herein is administered to the patient. This can be in conjunction with the administration of autologous or heterologous bone marrow or hematopoietic stem or progenitor cells, as well as other factors such as stem cell factor (SCF).

Alternatively, a patient may have “tired” bone marrow and may not be producing sufficient numbers of HSC and other blood cells to produce normal quantities. This can be caused by a variety of conditions, including normal aging, prolonged infection, post-chemotherapy, post-radiation therapy, chronic disease states including cancer, genetic abnormalities, and immunosuppression induced in transplantation. Further, radiation, such as whole-body radiation, can have a major impact on the bone marrow productivity. These conditions can also be either pre-treated to minimize the negative effects (such as for chemotherapy and/or radiation therapy, or treated after occurrence to reverse the effects.

### EXAMPLES

The following Examples provide specific examples of methods of the invention, and are not to be construed as limiting the invention to their content.

### Example 1

### Reversal of Aged-induced Thymic Atrophy

Materials and Methods

Animals. CBA/CAH and C57B16/J male mice were obtained from Central Animal Services, Monash University and were housed under conventional conditions. C57B16/J Ly5.1⁺ were obtained from the Central Animal Services Monash University, the Walterand Eliza Hall Institute for Medical Research (Parkville, Victoria) and the A.R.C. (Perth Western Australia) and were housed under conventional conditions. Ages ranged from 4-6 weeks to 26 months of age and are indicated where relevant.

Surgical castration. Animals were anesthetized by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline. Surgical castration was performed by a scrotal incision, revealing the testes, which were tied with suture and then removed along with surrounding fatty tissue. The wound was closed using surgical staples. Sham-castration followed the above procedure without removal of the testes and was used as controls for all studies.

Bromodeoxyuridine (BrdU) incorporation. Mice received two intraperitoneal injections of BrdU (Sigma Chemical Co., St. Louis, Mo) at a dose of 100 mg/kg body weight in 100 μl of PBS, 4-hours apart (i.e., at 4 hour intervals). Control mice received vehicle alone injections. One hour after the second injection, thymuses were dissected and either a cell suspension made for FACS analysis, or immediately embedded in Tissue Tek (O.C.T. compound, Miles INC, Indiana), snap frozen in liquid nitrogen, and stored at −70° C. until use.

Flow Cytometric analysis. Mice were killed by CO₂ asphyxiation and thymus, spleen, and mesenteric lymph nodes were removed. Organs were pushed gently through a 200 μm sieve in cold PBS/1% FCS/0.02% Azide, centrifuged (650 g, 5 min, 4° C.), and resuspended in either PBS/FCS/Az. Spleen cells were incubated in red cell lysis buffer (8.9 g/liter ammonium chloride) for 10 min at 4° C., washed and resuspended in PBS/FCS/Az. Cell concentration and viability were determined in duplicate using a hemocytometer and ethidium bromide/acridine orange and viewed under a fluorescence microscope (Axioskop; Carl Zeiss, Oberkochen, Germany).

For 3-color immunofluorescence, cells were labeled with anti-αβTCR-FITC, anti-CD4-PE and anti-CD8-APC (all obtained from Pharmingen, San Diego, Calif.) followed by flow cytometry analysis. Spleen and lymph node suspensions were labeled with either αβTCR-FITC/CD4-PE/CD8-APC or B220-B (Sigma) with CD4-PE and CD8-APC. B220-B was revealed with streptavidin-Tri-color conjugate purchased from Caltag Laboratories, Inc., Burlingame, Calif.

For BrdU detection of cells, cells were surface labeled with CD4-PE and CD8-APC, followed by fixation and permeabilization as previously described (Carayon and Bord, 1989). Briefly, stained cells were fixed overnight at 4° C. in 1% paraformaldehyde (PFA)/0.01% Tween-20. Washed cells were incubated in 500 μl DNase (100 Kunitz units, Roche, USA) for 30 mins at 37° C. in order to denature the DNA. Finally, cells were incubated with anti-BrdU-FITC (Becton-Dickinson) for 30 min at room temperature, washed and resuspended for FACS analysis.

For BrdU analysis of TN subsets, cells were collectively gated out on Lin- cells in APC, followed by detection for CD44-biotin and CD25-PE prior to BrdU detection. All antibodies were obtained from Pharmingen, USA.

For 4-color Immunofluorescence, thymocytes were labeled for CD3, CD4, CD8, B220 and Mac-1, collectively detected by anti-rat Ig-Cy5 (Amersham, U.K.), and the negative cells (TN) gated for analysis. They were further stained for CD25-PE (Pharmingen) and CD44-B (Pharmingen) followed by Streptavidin-Tri-colour (Caltag, Calif.) as previously described (Godfrey and Zlotnik, 1993). BrdU detection was then performed as described above.

Samples were analyzed on a FacsCalibur (Becton-Dickinson). Viable lymphocytes were gated according to 0° and 90° light scatter profiles and data was analyzed using Cell quest software (Becton-Dickinson).

Imumunohistology. Frozen thymus sections (4 μm) were cut using a cryostat (Leica) and immediately fixed in 100% acetone.

For two-color immunofluorescence, sections were double-labeled with a panel of monoclonal antibodies: MTS6, 10, 12, 15, 16, 20, 24, 32, 33, 35 and 44 (Godfrey et al., 1990; Table 1) produced in this laboratory and the co-expression of epithelial cell determinants was assessed with a polyvalent rabbit anti-cytokeratin Ab (Dako, Carpinteria, Calif.). Bound mAb was revealed with FITC-conjugated sheep anti-rat Ig (Silenus Laboratories) and anti-cytokeratin was revealed with TRITC-conjugated goat anti-rabbit Ig (Silenus Laboratories).

For BrdU detection of sections, sections were stained with either anti-cytokeratin followed by anti-rabbit-TRITC or a specific mAb, which was then revealed with anti-rat Ig-Cγ3 (Amersham). BrdU detection was then performed as previously described (Penit et al., 1996). Briefly, sections were fixed in 70% Ethanol for 30 mins. Semi-dried sections were incubated in 4M HCl, neutralized by washing in Borate Buffer (Sigma), followed by two washes in PBS. BrdU was detected using anti-BrdU-FITC (Becton-Dickinson).

For three-color immunofluorescence, sections were labeled for a specific MTS mAb together with anti-cytokeratin. BrdU detection was then performed as described above.

Sections were analyzed using a Leica fluorescent and Nikon confocal microscopes.

Migration studies (i.e., Analysis of recent thymic emigrants (RTIE)). Animals were anesthetized by intraperitoneal injection of 0.3 ml of 0.3 mg xylazine (Rompun; Bayer Australia Ltd., Botany NSW, Australia) and 1.5 mg ketamine hydrochloride (Ketalar; Parke-Davis, Caringbah, NSW, Australia) in saline.

Details of the FITC labeling of thymocytes technique are similar to those described elsewhere (Scollay et al., 1980; Berzins et al., 1998). Briefly, thymic lobes were exposed and each lobe was injected with approximately 10 μm of 350 μg/ml FITC (in PBS). The wound was closed with a surgical staple, and the mouse was warmed until fully recovered from anesthesia. Mice were killed by CO₂ asphyxiation approximately 24 hours after injection and lymphoid organs were removed for analysis.

After cell counts, samples were stained with anti-CD4-PE and anti-CD8-APC, then analyzed by flow cytometry. Migrant cells were identified as live-gated FITC⁺ cells expressing either CD4 or CD8 (to omit autofluorescing cells and doublets). The percentages of FITC⁺ CD4 and CD8 cells were added to provide the total migrant percentage for lymph nodes and spleen, respectively. Calculation of daily export rates was performed as described by Berzins et al., 1998).

Data analyzed using the unpaired student ‘t’ test or nonpararnetrical Mann-Whitney U-test was used to determine the statistical significance between control and test results for experiments performed at least in triplicate. Experimental values significantly differing from control values are indicated as follows: *p≦0.05, **p≦0.01 and ***p≦0.001.

### Results

I. The Effect of Age on Thymocyte Populations.

(i) Thymic Weight and Thymocyte Number

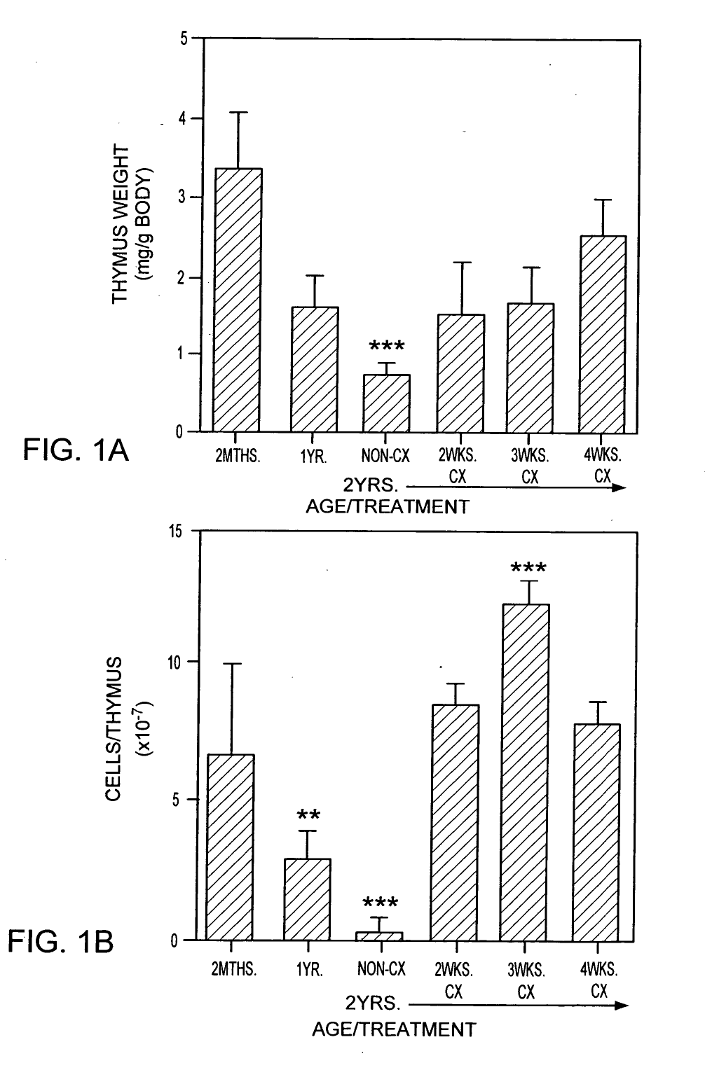

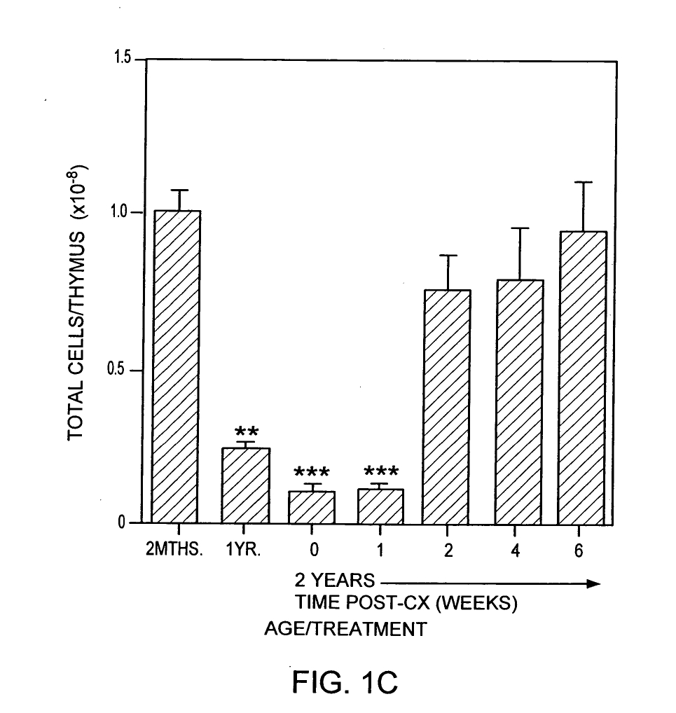

With increasing age there is a highly significant (p≦0.0001) decrease in both thymic weight (FIG. 1A) and total thymocyte number (FIGS. 1B and 1C) in mice. Relative thymic weight (mg thymus/g body) in the young adult has a mean value of 3.34 which decreases to 0.66 at 18-24 months of age (adipose deposition limits accurate calculation). The decrease in thymic weight can be attributed to a decrease in total thymocyte numbers: the 1-2 month (i.e., young adult) thymus contains ˜6.7˜10⁷ thymocytes, decreasing to ˜4.5×10⁶ cells by 24 months. By removing the effects of sex steroids on the thymus by castration, thymocyte cell numbers are regenerated and by 4 weeks post-castration, the thymus is equivalent to that of the young adult in both weight (FIG. 1A) and cellularity (FIGS. 1B and 1C). Interestingly, there was a significant (p≦0.001) increase in thymocyte numbers at 2 weeks post-castration (1.2×10⁸), which is restored to normal young levels by 4 weeks post-castration (FIG. 1B).

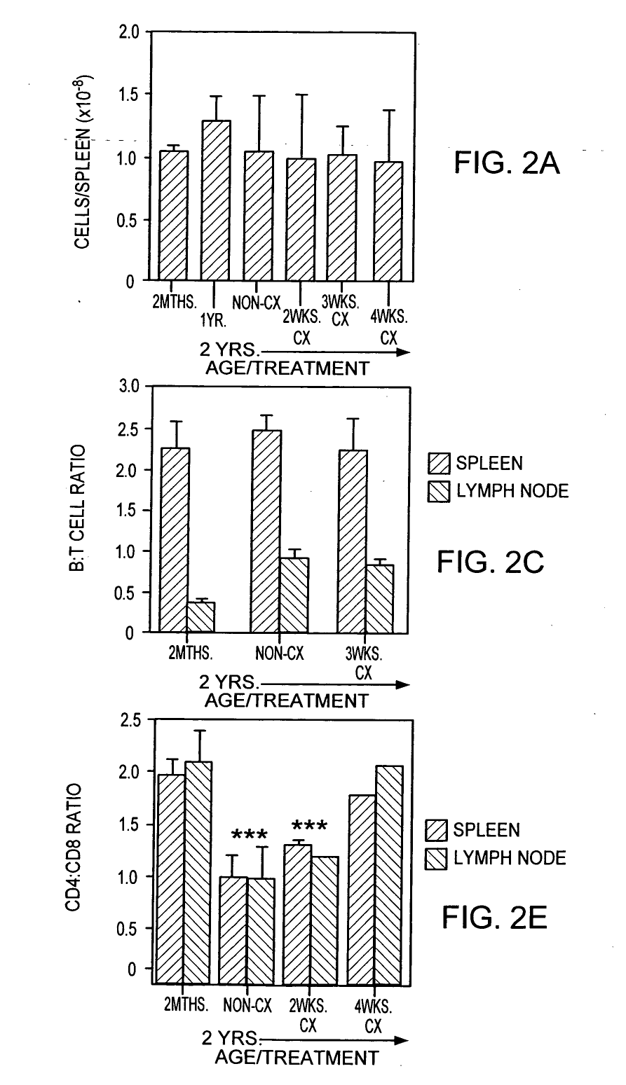

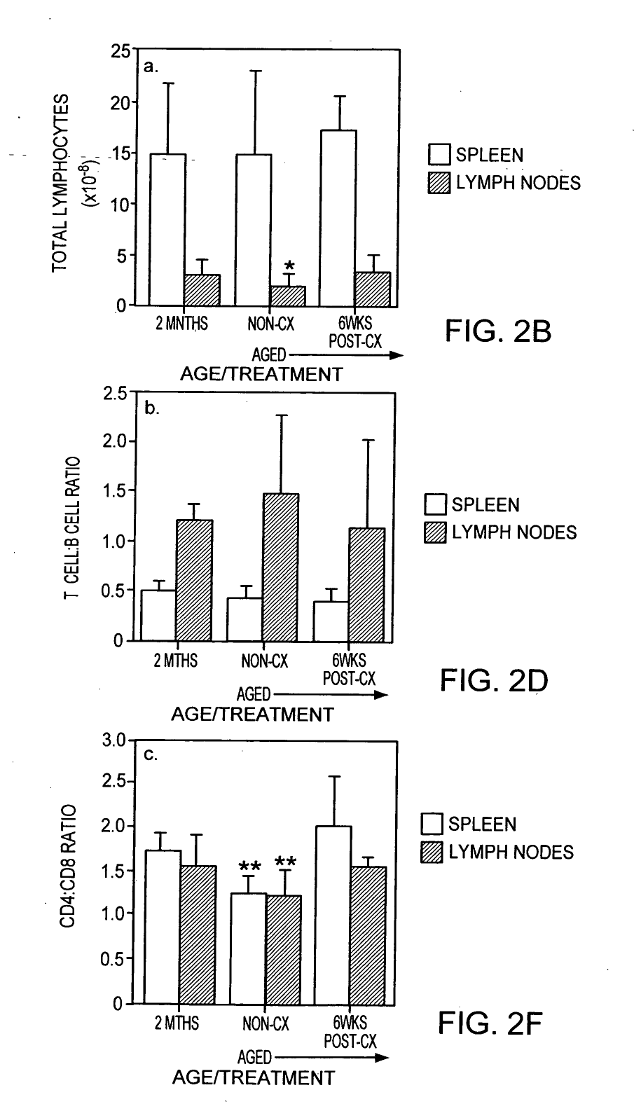

The decrease in T cell numbers produced by the thymus is not reflected in the periphery, with spleen cell numbers remaining constant with age (FIG. 2A and 2B). Homeostatic mechanisms in the periphery were evident since the B cell to T cell ratio in spleen and lymph nodes was not affected with age and the subsequent decrease in T cell numbers reaching the periphery (FIGS. 2C and 2D). However, the ratio of CD4⁺ to CD8⁺ T cell significantly decreased (p≦0.001) with age from 2:1 at 2 months of age, to a ratio of 1:1 at 2 years of age (FIGS. 2D and 2E). Following castration and the subsequent rise in T cell numbers reaching the periphery, no change in peripheral T cell numbers was observed: splenic T cell numbers and the ratio of B:T cells in both spleen and lymph nodes was not altered following castration (FIGS. 2A-2D). The reduced CD4:CD8 ratio in the periphery with age was still evident at 2 weeks post-castration but was completely reversed by 4 weeks post-castration (FIG. 2E)

(ii) Thymocyte Subpopulations with Age and Post-castration.

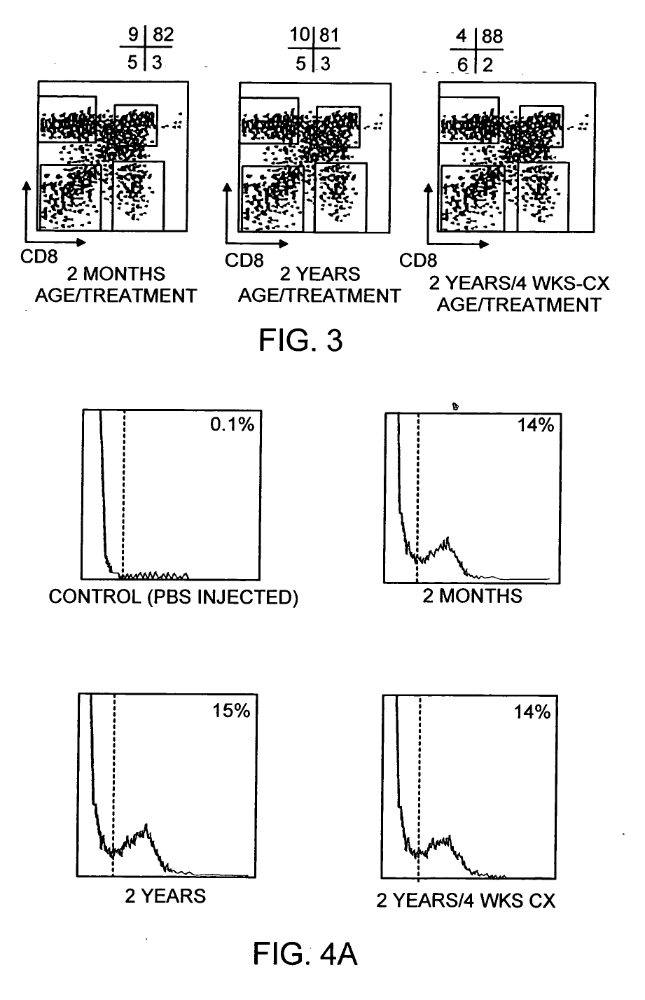

To determine if the decrease in thymocyte numbers seen with age was the result of the depletion of specific cell populations, thymocytes were labeled with defining markers in order to analyze the separate subpopulations. In addition, this allowed analysis of the kinetics of thymus repopulation post-castration. The proportion of the main thymocyte subpopulations was compared with those of the young adult (2-4 months) thymus (FIG. 3) and found to remain uniform with age. In addition, further subdivision of thymocytes by the expression of αβTCR revealed no change in the proportions of these populations with age (data not shown). At 2 and 4 weeks post-castration, thymocyte subpopulations remained in the same proportions and, since thymocyte numbers increase by up to 100-fold post-castration, this indicates a synchronous expansion of all thymocyte subsets rather than a developmental progression of expansion.

The decrease in cell numbers seen in the thymus of aged (2 year old) animals thus appears to be the result of a balanced reduction in all cell phenotypes, with no significant changes in T cell populations being detected. Thymus regeneration occurs in a synchronous fashion, replenishing all T cell subpopulations simultaneously rather than sequentially.

II. Proliferation of Thymocytes

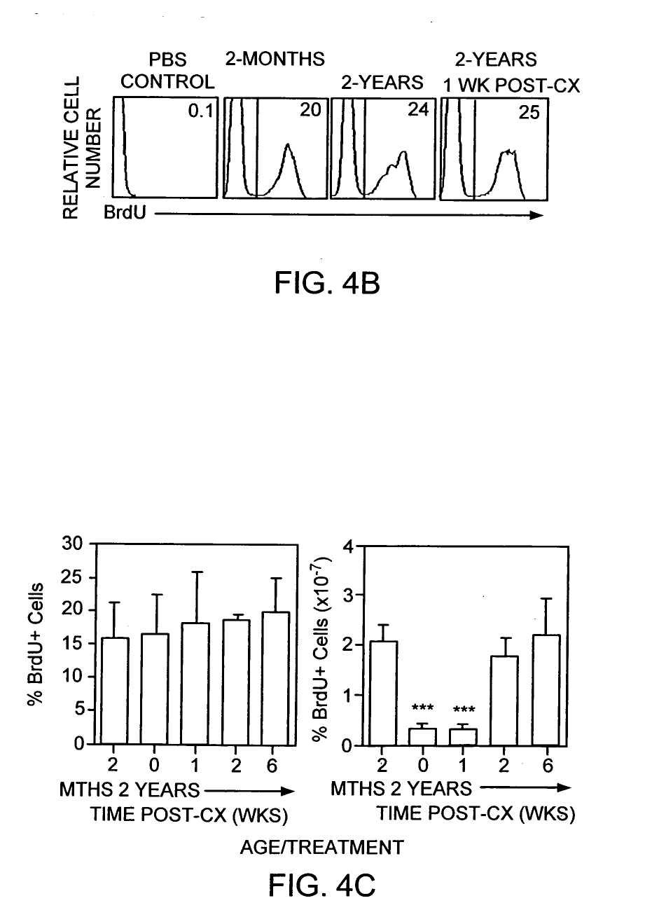

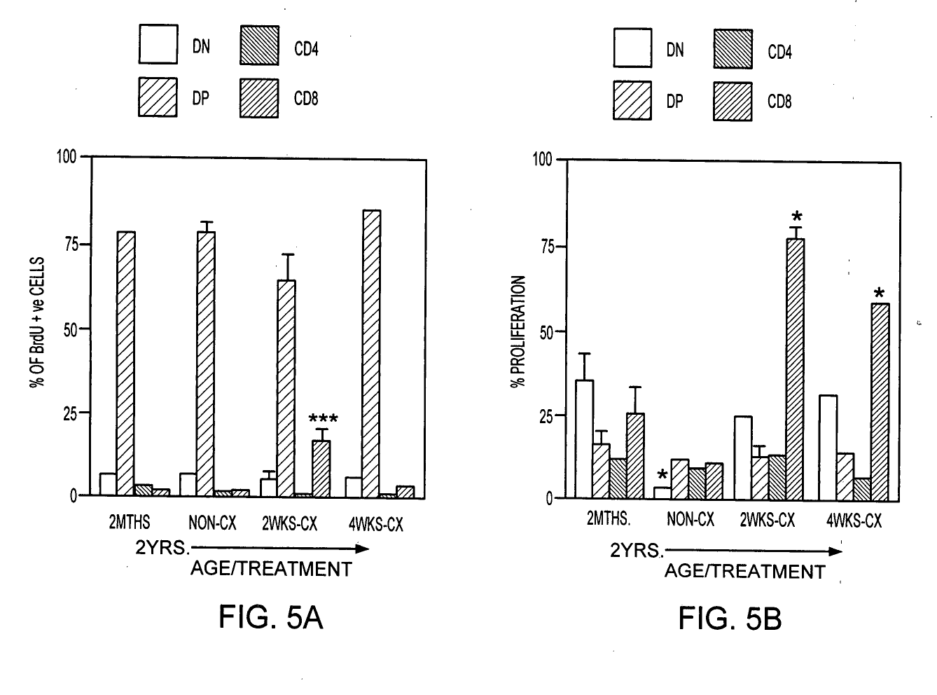

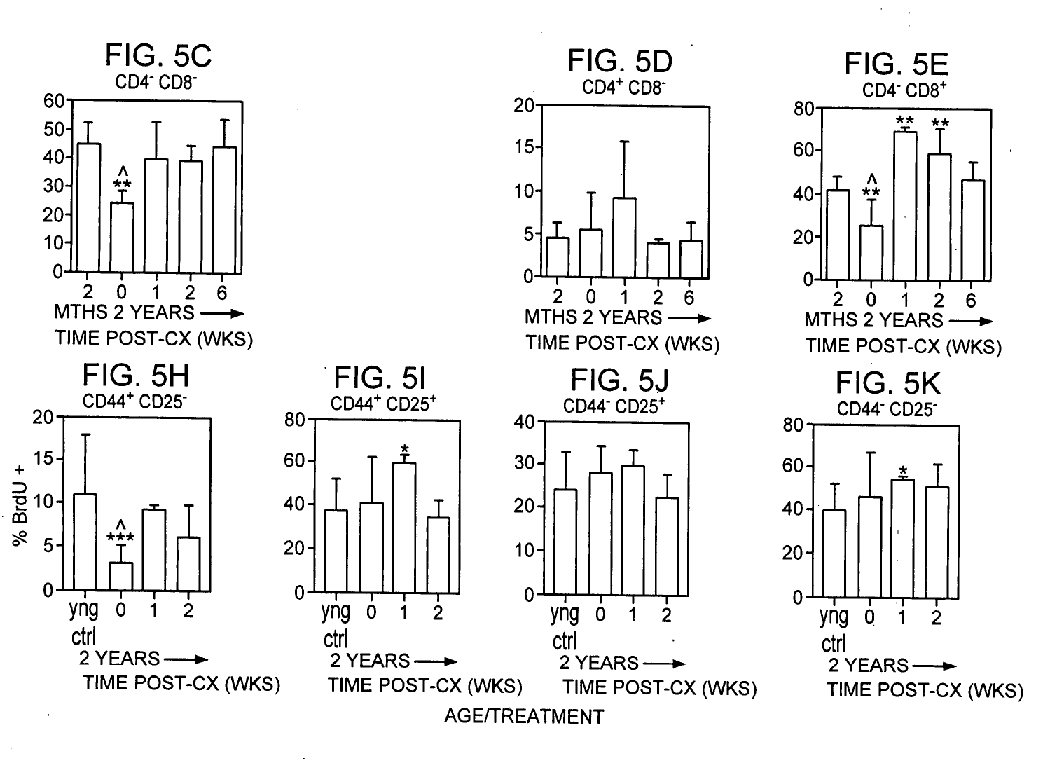

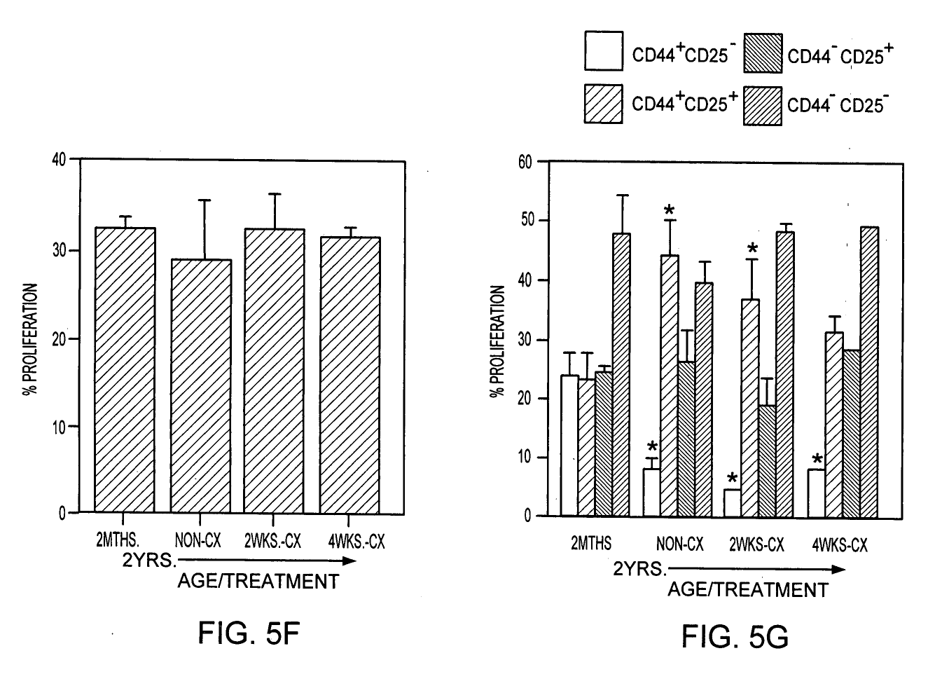

As shown in FIGS. 4A-4C, 15-20% of thymocytes were proliferating at 2-4 months of age. The majority (˜80%) of these are double positive (DP) (i.e., CD4+, CD8+) with the triple negative (TN) (i.e., CD3−CD4−CD8−) subset making up the second largest population at ˜6% (FIGS. 5A). These TN cells are the most immature cells in the thymus and encompass the intrathymic precursor cells. Accordingly, most division is seen in the subcapsule and cortex by immunohistology (data not shown). Some division is seen in the medullary regions aligning with FACS analysis which revealed a proportion of single positive (i.e., CD4+CD8− or CD4−CD8+) cells (9% of CD4+ T cells and 25% of CD8+ T cells) in the young (2 months) thymus, dividing (FIG. 5B).

Although cell numbers were significantly decreased in the aged mouse thymus (2 years old), the total proportion of proliferating thymocytes remained constant (FIGS. 4C and SF), but there was a decrease in the proportion of dividing cells in the CD4−CD8− (FIG. 5C) and proliferation of CD4−CD8+ T cells was also significantly (p≦0.001) decreased (FIG. 5E). Immunohistology revealed the distribution of dividing cells at 1 year of age to reflect that seen in the young adult (2-4 months); however, at 2 years, proliferation is mainly seen in the outer cortex and surrounding the vasculature with very little division in the medulla (data not shown).

As early as one week post-castration there was a marked increase in the proportion of proliferating CD4−CD8− cells (FIG. 5C) and the CD4−CD8+ cells (FIG. 5E). Castration clearly overcomes the block in proliferation of these cells with age. There was a corresponding proportional decrease in proliferating CD4+CD8− cells post-castration (FIG. 5D). At 2 weeks post-castration, although thymocyte numbers significantly increase, there was no change in the overall proportion of thymocytes that were proliferating, again indicating a synchronous expansion of cells (FIGS. 4A, 4B, 4C and 5F). Immunohistology revealed the localization of thymocyte proliferation and the extent of dividing cells to resemble the situation in the 2-month-old thymus by 2 weeks post-castration (data not shown).

The DN subpopulation, in addition to the thymocyte precursors, contains (αβTCR+CD4−CD8− thymocytes, which are thought to have downregulated both co-receptors at the transition to SP cells (Godfrey & Zlotnik, 1993). By gating on these mature cells, it was possible to analyze the true TN compartment (CD3⁻CD4⁻CD8⁻) and their subpopulations expressing CD44 and CD25. FIGS. 5H, 5I, 5J, and 5K illustrate the extent of proliferation within each subset of TN cells in young, old and castrated mice. This showed a significant (p<0.001) decrease in proliferation of the TN1 subset (CD44⁺CD25⁻CD3⁻CD4⁻CD8⁻), from ˜10%% in the normal young to around 2% at 18 months of age (FIG. 5H) which was restored by 1 week post-castration.

III. The Effect of Age on the Thymic Microenvironment.

The changes in the thymic microenvironment with age were examined by immunofluorescence using an extensive panel of MAbs from the MTS series, double-labeled with a polyclonal anti-cytokeratin Ab.

The antigens recognized by these MAbs can be subdivided into three groups: thymic epithelial subsets, vascular-associated antigens and those present on both stromal cells and thymocytes.

(i) Epithelial Cell Antigens.

Anti-keratin staining (pan-epithelium) of 2 year old mouse thymus, revealed a loss of general thymus architecture with a severe epithelial cell disorganization and absence of a distinct cortico-medullary junction. Further analysis using the MAbs, MTS 10 (medulla) and MTS44 (cortex), showed a distinct reduction in cortex size with age, with a less substantial decrease in medullary epithelium (data not shown). Epithelial cell free regions, or keratin negative areas (KNA's, van Ewijk et al., 1980; Godfrey et al., 1990; Bruijntjes et al., 1993) were more apparent and increased in size in the aged thymus, as evident with anti-cytokeratin labeling. There was also the appearance of thymic epithelial “cyst-like” structures in the aged thymus particularly noticeable in medullary regions (data not shown). Adipose deposition, severe decrease in thymic size and the decline in integrity of the cortico-medullary junction were shown conclusively with the anti-cytokeratin staining (data not shown). The thymus began to regenerate by 2 weeks post-castration. This was evident in the size of the thymic lobes, the increase in cortical epithelium as revealed by MTS 44, and the localization of medullary epithelium. The medullary epithelium was detected by MTS 10 and at 2 weeks, there are still subpockets of epithelium stained by MTS 10 scattered throughout the cortex. By 4 weeks post-castration, there was a distinct medulla and cortex and discernible cortico-medullary junction (data not shown).

The markers MTS 20 and 24 are presumed to detect primordial epithelial cells (Godfrey et al., 1990) and further illustrate the degeneration of the aged thymus. These were present in abundance at E14, detect isolated medullary epithelial cell clusters at 4-6 weeks but were again increased in intensity in the aged thymus (data not shown). Following castration, all these antigens were expressed at a level equivalent to that of the young adult thymus (data not shown) with MTS 20 and MTS 24 reverting to discrete subpockets of epithelium located at the cortico-medullary junction.

(ii) Vascular-associated Antigens.

The blood-thymus barrier is thought to be responsible for the immigration of T cell precursors to the thymus and the emigration of mature T cells from the thymus to the periphery.

The mAb MTS 15 was specific for the endothelium of thymic blood vessels, demonstrating a granular, diffuse staining pattern (Godfrey et al., 1990). In the aged thymus, MTS 15 expression was greatly increased, and reflects the increased frequency and size of blood vessels and perivascular spaces (data not shown).

The thymic extracellular matrix, containing important structural and cellular adhesion molecules such as collagen, laminin and fibrinogen, was detected by the mAb MTS 16. Scattered throughout the normal young thymus, the nature of MTS 16 expression becomes more widespread and interconnected in the aged thymus. Expression of MTS 16 was increased further at 2 weeks post-castration while at 4 weeks post-castration, this expression is representative of the situation in the 2 month thymus (data not shown).

(iii) Shared Antigens

MHC II expression in the normal young thymus, detected by the MAb MTS 6, was strongly positive (granular) on the cortical epithelium (Godfrey et al., 1990) with weaker staining of the medullary epithelium. The aged thymus showed a decrease in MHC II expression with expression substantially increased at 2 weeks post-castration. By 4 weeks post-castration, expression was again reduced and appeared similar to the 2 month old thymus (data not shown).

IV. Thymocyte Emigration

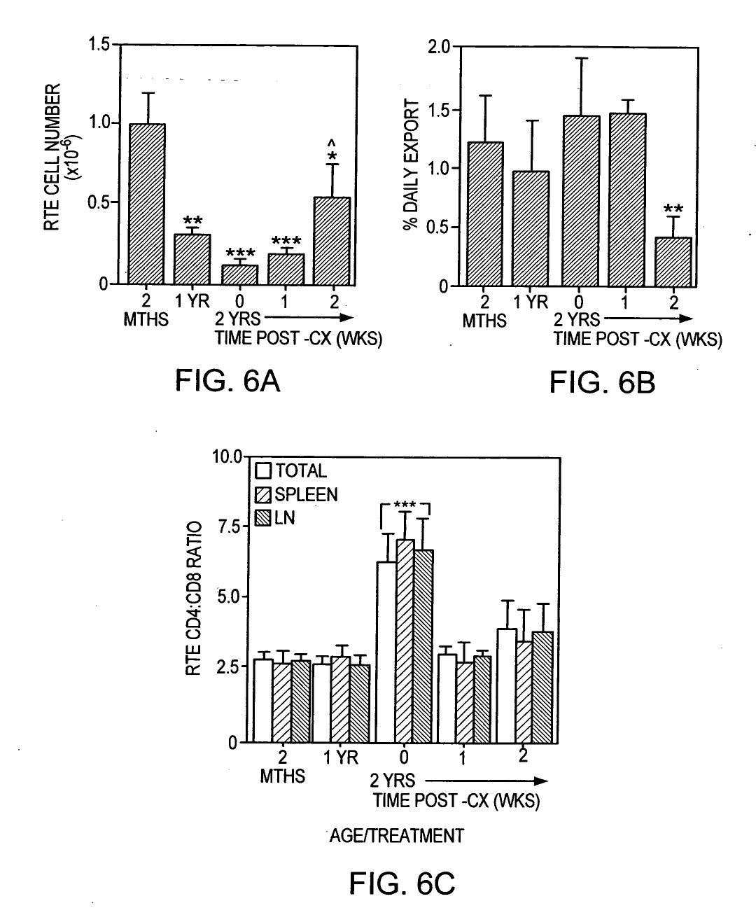

Approximately 1% of T cells migrate from the thymus daily in the young mouse (Scollay et al., 1980). Migration in castrated mice was found to occur at a proportional rate equivalent to the normal young mouse at 14 months and even 2 years of age, although significantly (p≦0.0001) reduced in number (FIGS. 6A and 6B). There was an increase in the CD4:CD8 ratio of the recent thymic emigrants from ˜3:1 at 2 months to ˜7:1 at 26 months (FIG. 6C). By 1 week post-castration, this ratio had normalised (FIG. 6C). By 2- weeks post-castration, cell number migrating to the periphery had substantially increased, with the overall rate of migration reduced to 0.4%, which reflected the expansion of the thymus (FIG. 6B).

By 2-weeks post-castration, a significant increase in RTE was observed (p≦0.01) compared to the aged mice. Despite the changes in cell numbers emigrating, the rate of emigration (RTE/total thymocytes) remained constant with age (FIG. 5b). However, at 2-weeks post-castration this had significantly decreased (p≦0.05), reflecting the increase in total thymocyte numbers at this time. Interestingly, there was an increase in the CD4:GD8 ratio of the RTE from ˜3:1 at 2 months to ˜7:1 at 26 months (FIG. 6C). By 1 week post-castration, this ratio had normalized (FIG. 6C).

V. Castration Induces Tolerance to Allograft (ie., Allogeneic Graft)

The following mice are purchased from the Jackson Laboratory (Bar Harbor, Me.), and are housed under conventional conditions: C57BL/6J (black; H-2b); DBA/1J (dilute brown; H-2q); DBA/2J (dilute brown; H-2d); and Balb/cJ (albino; H-2d). Ages range from 4-6 weeks to 26 months of age and are indicated where relevant.

C57BL/6J mice are used as recipients for donor bone marrow reconstitution. As described above, the recipient mice (C57BL6/J older than 9 months of age, because this is the age at which the thymus has begun to markedly atrophy) are subjected to 5.5Gy irradiation twice over a 3-hour interval. One hour following the second irradiation dose, the recipient mice are injected intravenously with 5×10⁶ donor bone marrow cells from DBA/1J, DBA/2J, or Balb/cJ mice. Bone marrow cells are obtained by passing RPMI-1640 media through the tibias and femurs of donor (2-month old DBA/1J, DBA/2J, or Balb/cJ) mice, and then harvesting the cells collected in the media.

As described above, in recipient mice castrated either at the same time as the reconstitution or up to one week prior to reconstitution, there is an significant increase in the rate of thymus regeneration compared to sham-castrated (ShCx) control mice. In addition, as compared to the sham-castrated mice, castrated mice are found to have increased thymus cellularity, have more cells in their bone marrow, and have enhanced generation of B cell precursors and B cells in their bone marrow following bone marrow transplantation. Since the MHC (i.e., the H-2 locus in mice) of the recipient mice is different from that of the donor mice, detecting an increased number of donor-derived blood cells in castrated mice as compared to sham-castrated mice is straightforward. There is also the normal level and distribution of host and donor-derived dendritic cells in the chimeric thymus which are exerting negative selection (tolerance induction) to the host and donor.

Four to six weeks after reconstitution of the recipient mice with donor bone marrow cells, skin grafts are taken from the donor mice and placed onto the recipient mice, according to standard methods (see, e.g., Unit 4.4 in Current Protocols In Immunology, John E. Coligan et al. (eds), Wiley and Sons, New York, N.Y. 1994, and yearly updates including 2002). Briefly, the dermis and epidermis of an anesthetized recipient mouse (e.g., a C57BL/6J mouse reconstituted with Balb/cJ bone marrow) are removed and replaced with the dermis and epidermis from a Balb/cJ. Because the hair of the donor skin is white, it is easily distinguished from the native black hair of the recipient C57BL/6J mouse. The health of the transplanted donor skin is assessed daily after surgery.

The results will show that donor Balb/cJ skin transplanted onto a donor-reconstituted C57BL/6J mouse who has been castrated “takes” (i.e., is accepted) better than the donor skin transplanted onto a donor-reconstituted C57BL/6J mouse who is sham-castrated, e.g., because the sham-castrated mouse does not have adequate uptake of donor HSC into the host thymus to produce DC. A donor skin graft is found not to take on a recipient, sham-castrated, C57BL/6J mouse who has not been reconstituted with Balb/cJ bone marrow.

An experiment is also performed to determine if a recipient mouse transplanted with donor bone marrow can induce tolerance of a MHC matched, but otherwise different, skin graft. Briefly, male C57BL/6J mice (H-2b) are either castrated or sham-castrated. The next day, the mice are reconstituted with Balb/cJ bone marrow (H-2d) as described above. Four weeks after reconstitution, two skin grafts (i.e., including the dermis and epidermis) are placed onto the recipient C57BL/6J mice. The first skin graft is from a DBA/2J (dilute brown; H-2d) mouse. The second skin graft is from a Balb/cJ mouse (albino; H-2d). Because the coat colors of C57BL/6J mice, Balb/cJ mice, and DBA/2J mice all differ, the skin grafts are easily distinguishable from one another and from the recipient mouse.

As described above, the skin graft from the Balb/cJ mouse is found to “take” onto the Balb/cJ-bone marrow reconstituted castrated recipient mouse better than a Balb/cJ-bone marrow reconstituted sham-castrated recipient mouse or a recipient mouse who has been sham-castrated and has not been reconstituted with donor bone marrow. In addition, the skin graft from the DBA/2J mouse is found to “take” onto the Balb/cJ-bone marrow reconstituted castrated recipient mouse better than a Balb/cJ-bone marrow reconstituted sham-castrated recipient mouse or a recipient mouse who has been sham-castrated and has not been reconstituted with donor bone marrow.

### Discussion

The present disclosure shows that aged thymus, although severely atrophic, maintains its functional capacity with age, with T cell, proliferation, differentiation and migration occurring at levels equivalent to the young adult mouse. Although thymic function is regulated by several complex interactions between the neuro-endocrine-immune axes, the atrophy induced by sex steroid production exerts the most significant and prolonged effects illustrated by the extent of thymus regeneration post-castration both of lymphoid and epithelial cell subsets.

Thymus weight is significantly reduced with age as shown previously (Hirokawa and Makinodan, 1975, Aspinall, 1997) and correlates with a significant decrease in thymocyte numbers. The stress induced by the castration technique, which may result in further thymus atrophy due to the actions of corticosteroids, is overridden by the removal of sex steroid influences with the 2-week castrate thymus increasing in cellularity by 20-30 fold from the pre-castrate thymus. By 3 weeks post-castration, the aged thymus shows a significant increase in both thymic size and cell number, surpassing that of the young adult thymus presumably due to the actions of sex steroids already exerting themselves in the 2 month old mouse.

The data presented herein confirms previous findings that emphasise the continued ability of thymocytes to differentiate and maintain constant subset proportions with age (Aspinall, 1997). In addition, thymocyte differentiation was found to occur simultaneously post-castration indicative of a synchronous expansion in thymocyte subsets. Since thymocyte numbers are decreased significantly with age, proliferation of thymocytes was analyzed to determine if this was a contributing factor in thymus atrophy.

Proliferation of thymocytes was not affected by age-induced thymic atrophy or by removal of sex-steroid influences post-castration with ˜14% of all thymocytes proliferating. However, the localization of this division differed with age: the 2 month mouse thymus shows abundant division throughout the subcapsular and cortical areas (TN and DP T cells) with some division also occurring in the medulla. Due to thymic epithelial disorganization with age, localization of proliferation was difficult to distinguish but appeared to be less uniform in pattern than the young and relegated to the outer cortex. By 2 weeks post-castration, dividing thymocytes were detected throughout the cortex and were evident in the medulla with similar distribution to the 2 month thymus.

The phenotype of the proliferating population as determined by CD4 and CD8 analysis, was not altered with age or following castration. However, analysis of proliferation within thymocyte subpopulations, revealed a significant decrease in proliferation of both the TN and CD8⁺ cells with age. Further analysis within the TN subset on the basis of the markers CD44 and CD25, revealed a significant decrease in proliferation of the TN1 (CD44⁺CD25⁻) population which was compensated for by an increase in the TN2 (CD44⁻CD25⁺) population. These abnormalities within the TN population, reflect the findings by Aspinall (1997). Surprisingly, the TN subset was proliferating at normal levels by 2 weeks post-castration indicative of the immediate response of this population to the inhibition of sex-steroid action. Additionally, at both 2 weeks and 4 weeks post-castration, the proportion of CD8⁺ T cells that were proliferating was markedly increased from the control thymus, possibly indicating a role in the reestablishment of the peripheral T cell pool.

Thymocyte migration was shown to occur at a constant proportion of thymocytes with age conflicting with previous data by Scollay et al. (1980) who showed a ten-fold reduction in the rate of thymocyte migration to the periphery. The difference in these results may be due to the difficulties in intrathymic FITC labelling of 2 year old thymuses or the effects of adipose deposition on FITC, uptake. However, the absolute numbers of T cells migrating was decreased significantly as found by Scollay resulting in a significant reduction in ratio of RTEs to the peripheral T cell pool. This will result in changes in the periphery predominantly affecting the T cell repertoire (Mackall et al., 1995). Previous papers (Mackall et al., 1995) have shown a skewing of the T cell repertoire to a memory rather than naive T cell phenotype with age. The diminished T cell repertoire however, may not cope if the individual encounters new pathogens, possibly accounting for the rise in immunodeficiency in the aged. Obviously, there is a need to reestablish the T cell pool in immunocompromised individuals. Castration allows the thymus to repopulate the periphery through significantly increasing the production of naive T cells.

In the periphery, T cell numbers remained at a constant level as evidenced in the B:T cell ratios of spleen and lymph nodes, presumably due to peripheral homeostasis (Mackall et al., 1995; Berzins et al., 1998). However, disruption of cellular composition in the periphery was evident with the aged thymus showing a significant decrease in CD4:CD8 ratios from 2:1 in the young adult to 1:1 in the 2 year mouse, possibly indicative of the more susceptible nature of CD4⁺ T cells to age or an increase in production of CD8+ T cells from extrathymic sources. By 2 weeks post-castration, this ratio has been normalized, again reflecting the immediate response of the immune system to surgical castration.

The above findings have shown firstly that the aged thymus is capable of functioning in a nature equivalent to the pre-pubertal thymus. In this respect, T cell numbers are significantly decreased but the ability of thymocytes to differentiate is not disturbed. Their overall ability to proliferate and eventually migrate to the periphery is again not influenced by the age-associated atrophy of the thymus. However, two important findings were noted. Firstly, there appears to be an adverse affect on the TN cells in their ability to proliferate, correlating with findings by Aspinall (1997). This defect could be attributed to an inherent defect in the thymocytes themselves. Yet the data presented herein and previous work has shown thymocyte differentiation, although diminished, still occurs and stem cell entry from the BM is also not affected with age (Hirokawa, 1998; Mackall and Gress, 1997). This implicates the thymic stroma as the target for sex steroid action and consequently abnormal regulation of this precursor subset of T cells. Secondly, the CD8⁺ T cells were significantly diminished in their proliferative capacity with age and, following castration, a significantly increased proportion of CD8⁺ T cells proliferated as compared to the 2 month mouse. The proliferation of mature T cells is thought to be a final step before migration (Suda and Zlotnik, 1992), such that a significant decrease in CD8⁺ proliferation would indicate a decrease in their migrational potential. This hypothesis is supported by our finding that the ratio of CD4:CD8 T cells in RTEs increased with age, indicative of a decrease in CD8 T cells migrating. Alternatively, if the thymic epithelium is providing the key factor for the CD8 T cell maintenance, whether a lymphostromal molecule or cytokine influence, this factor may be disturbed with increased sex-steroid production. By removing the influence of sex-steroids, the CD8 T cell population can again proliferate optimally. Thus, it was necessary to determine, in detail, the status of thymic epithelial cells pre- and post-castration.

The cortex appears to ‘collapse’ with age due to lack of thymocytes available to expand the network of epithelium. The most dramatic change in thymic epithelium post-castration was the increased network of cortical epithelium detected by MTS 44, illustrating the significant rise in thymocyte numbers. At 2 weeks post-castration, KNAs are abundant and appear to accommodate proliferating thymocytes indicating that thymocyte development is occurring at a rate higher than the epithelium can cope with. The increase in cortical epithelium appears to be due to stretching of the thymic architecture rather than proliferation of this subtype since no proliferation of the epithelium was noted with BrdU staining by immunofluorescence.

Medullary epithelium is not as susceptible to age influences most likely due to the lesser number of T cells accumulating in this area (>95% of thymocytes are lost at the DP stage due to selection events). However, the aged thymus shows severe epithelial cell disruption distinguished by a lack of distinction of the cortico-medullary junction with the medullary epithelium incorporating into the cortical epithelium. By 2 weeks postcastration, the medullary epithelium, as detected by MTS 10 staining is reorganized to some extent, however, subpockets are still present within the cortical epithelium. By 4 weeks post-castration, the cortical and medullary epithelium is completely reorganized with a distinct cortico-medullary junction similar to the young adult thymus.

Subtle changes were also observed following castration, most evident in the decreased expression of MHC class II and blood-thymus barrier antigens when compared to the pre-castrate thymus. MHCII (detected by MTS6) is increased in expression in the aged thymus possibly relating to a decrease in control by the developing thymocytes due to their diminished numbers. Alternatively, it may simply be due to lack of masking by the thymocytes, illustrated also in the post-irradiation thymus (Randle and Boyd, 1992) which is depleted of the DP thymocytes. Once thymocyte numbers are increased following castration, the antigen binding sites are again blocked by the accumulation of thymocytes thus decreasing detection by immunofluorescence. The antigens detecting the blood-thymus barrier (MTS12, 15 and 16) are again increased in the aged thymus and also revert to the expression in the young adult thymus post-castration. Lack of masking by thymocytes and the close proximity of the antigens due to thymic atrophy may explain this increase in expression. Alternatively, the developing thymocytes may provide the necessary control mechanisms over the expression of these antigens thus when these are depleted, expression is not controlled. The primordial epithelial antigens detected by MTS 20 and NITS 24 are increased in expression in the aged thymus but revert to subpockets of epithelium at the cortico-medullary junction post-castration. This indicates a lack of signals for this epithelial precursor subtype to differentiate in the aged mouse. Removing the block placed by the sex-steroids, these antigens can differentiate to express cortical epithelial antigens.

The above findings indicate a defect in the thymic epithelium rendering-it incapable of providing the developing thymocytes with the necessary stimulus for, development. However, the symbiotic nature of the thymic, epithelium and thymocytes makes it difficult to ascertain the exact pathway of destruction by the sex steroid influences. The medullary epithelium requires cortical T cells for its proper development and maintenance. Thus, if this population is diminished, the medullary thymocytes may not receive adequate signals for development. This particularly seems to affect the CD8⁺ population. IRF^{-/-} mice show a decreased number of CD8⁺ T cells. It would therefore, be interesting to determine the proliferative capacity of these cells.

The defect in proliferation of the TN1 subset which was observed indicates that loss of cortical epithelium affects thymocyte development at the crucial stage of TCR gene rearrangement whereby the cortical epithelium provides factors such as IL-7 and SCF necessary for thymopoiesis (Godfrey and Zlotnik, 1990; Aspinall, 1997). Indeed, IL-7^{-/-} and IL-7R^{-/-} mice show similar thymic morphology to that seen in aged mice (Wiles et al., 1992; Zlotnik and Moore, 1995; von Freeden-Jeffry, 1995). Further work is necessary to determine the changes in IL-7 and IL-7R with age.

In conclusion, the aged thymus still maintains its functional capacity, however, the thymocytes that develop in the aged mouse are not under the stringent control by thymic epithelial cells as seen in the normal young mouse due to the lack of structural integrity of the thymic microenvironment. Thus the proliferation, differentiation and migration of these cells will not be under optimal regulation and may result in the increased release of autoreactive/immunodysfunctional T cells in the periphery. The defects within both the TN and particularly, CD8⁺ populations, may result in the changes seen within the peripheral T cell pool with age. In addition, as described in detail herein, the effects of castration on thymic epithelial cell development and reorganization. The mechanisms underlying thymic atrophy utilizing steroid receptor binding assays and the role of thymic epithelial subsets in thymus regeneration post-castration are currently under study. Restoration of thymus function by castration will provide an essential means for regenerating the peripheral T cell pool and thus in-re-establishing immunity in immunosuppressed individuals.

### Example 2

### Reversal of Chemotherapy- or Radiation-induced Thymic Atrophy

Materials and methods were as described in Example 1. In addition, the following methods were used.

Bone Marrow reconstitution. Recipient mice (3-4 month-old C57BL6/J) were subjected to 5.5Gy irradiation twice over a 3-hour interval. One hour following the second irradiation dose, mice were injected intravenously with 5×10⁶ donor bone marrow cells. Bone marrow cells were obtained by passing RPMI-1640 media through the tibias and femurs of donor (2-month old congenic C57BL6/J Ly5.1⁺) mice, and then harvesting the cells collected in the media.

T cell Depletion Using Cyclophosphamide

Old mice (e.g., 2 years old) were injected with cyclophosphamide (200 mg/kg body wt) and castrated on the same day.

HSV-1 immunization. Following anesthetic, mice were injected in the foot-hock with 4×10⁵ plaque forming units (pfu) of HSV-1 in sterile PBS. Analysis of the draining (popliteal) lymph nodes was performed on D5 post-infection.

For HSV-1 studies, popliteal lymph node cells were stained for anti-CD25-PE, anti-CD8-APC and anti-Vβ10-biotin. For detection of dendritic cells, an FcR block was used prior to staining for CD45.1-FITC, I-A^{b}-PE and CD11c-biotin. All biotinylated antibodies were detected with streptavidin-PerCP. For detection of HSC, BM cells were gated on Lin- cells by collectively staining with anti-CD3, CD4, CD8, Gr-1, B220 and Mac-1 (all conjugated to FITC). HSC were detected by staining with CD117-APC and Sca-1-PE. For TN thymocyte analysis, cells were gated on the Lin⁻ population and detected by staining with CD44-biotin, CD25-PE and c-kit-APC.

Cytotoxicity assay of lymph node cells. Lymph node cells were incubated for three days at 37° C., 6.5% CO₂. Specificity was determined using a non-transfected cell line (EL4) pulsed with gB₄₉₈₋₅₀₅ peptide (gBp) and EL4 cells alone as a control. A starting effector:target ratio of 30:1 was used. The plates were incubated at 37° C., 6.5% CO₂ for four hours and then centrifuged 650_{gmax} for 5 minutes. Supernatant (100 μl) was harvested from each well and transferred into glass fermentation tubes for measurement by a Packard Cobra auto-gamma counter.

Castration enhanced regeneration following severe T cell depletion (TCD).

Castrated mice (castrated either one-week prior to treatment, or on the same day as treatment), showed substantial increases in thymus regeneration rate following irradiation or cyclophosphamide treatment.

In the thymus, irradiated mice showed severe disruption of thymic architecture, concurrent with depletion of rapidly dividing cells. Cortical collapse, reminiscent of the aged/hydrocortisone treated thymus, revealed loss of DN and DP thymocytes. There was a downregulation of αβ-TCR expression on CD4+ and CD8+ SP thymocytes—evidence of apoptosing cells. In comparison, cyclophosphamide-treated animals show a less severe disruption of thymic architecture, and show a faster regeneration rate of DN and DP thymocytes.

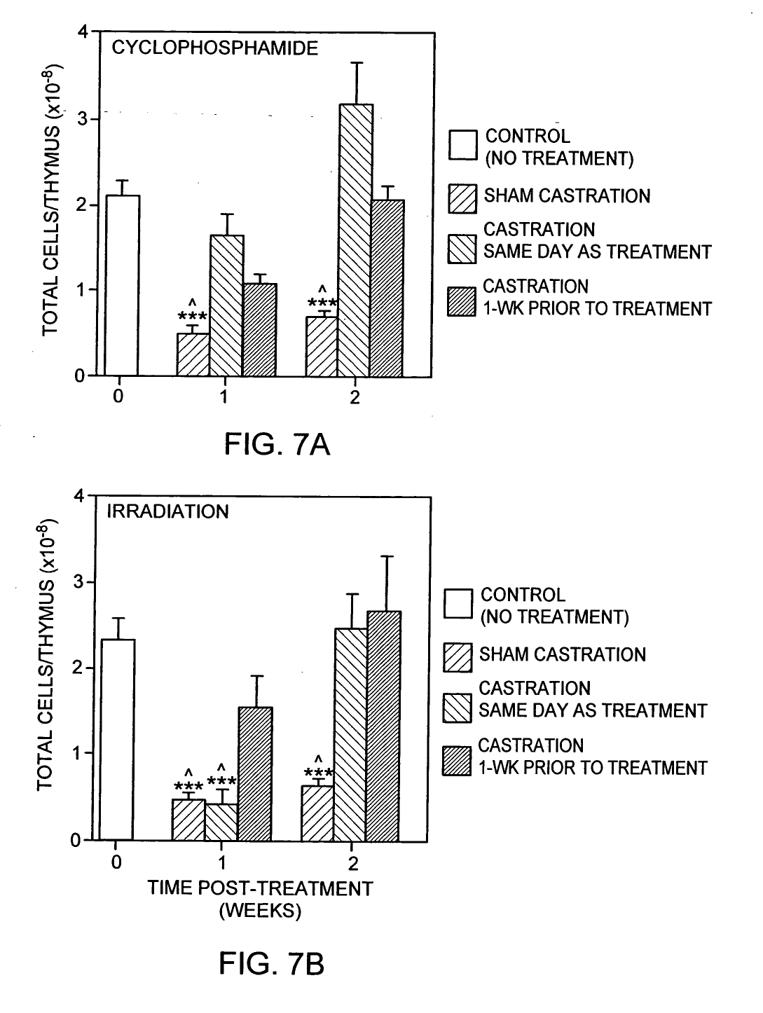

For both models of T cell depletion studied (chemotherapy using cyclolphosphamide or sublethal irradiation using 625 Rads), castrated (Cx) mice showed a significant increase in the rate of thymus regeneration compared to their sham-castrated (ShCx) counterparts (FIGS. 7A and 7B). By 1 week post-treatment castrated mice showed significant thymic regeneration even at this early stage (FIGS. 7, 8, 10, 11, and 12). In comparison, non-castrated animals, showed severe loss of DN and DP thymocytes (rapidly-dividing cells) and subsequent increase in proportion of CD4 and CD8 cells (radio-resistant). This is best illustrated by the differences in thymocyte numbers with castrated animals showing at least a 4-fold increase in thymus size even at 1 week post-treatment. By 2 weeks, the non-castrated animals showed relative thymocyte normality with regeneration of both DN and DP thymocytes. However, proportions of thymocytes are not yet equivalent to the young adult control thymus. Indeed, at 2 weeks, the vast difference in regulation rates between castrated and non-castrated mice was maximal (by 4 weeks thymocyte numbers were equivalent between treatment groups).

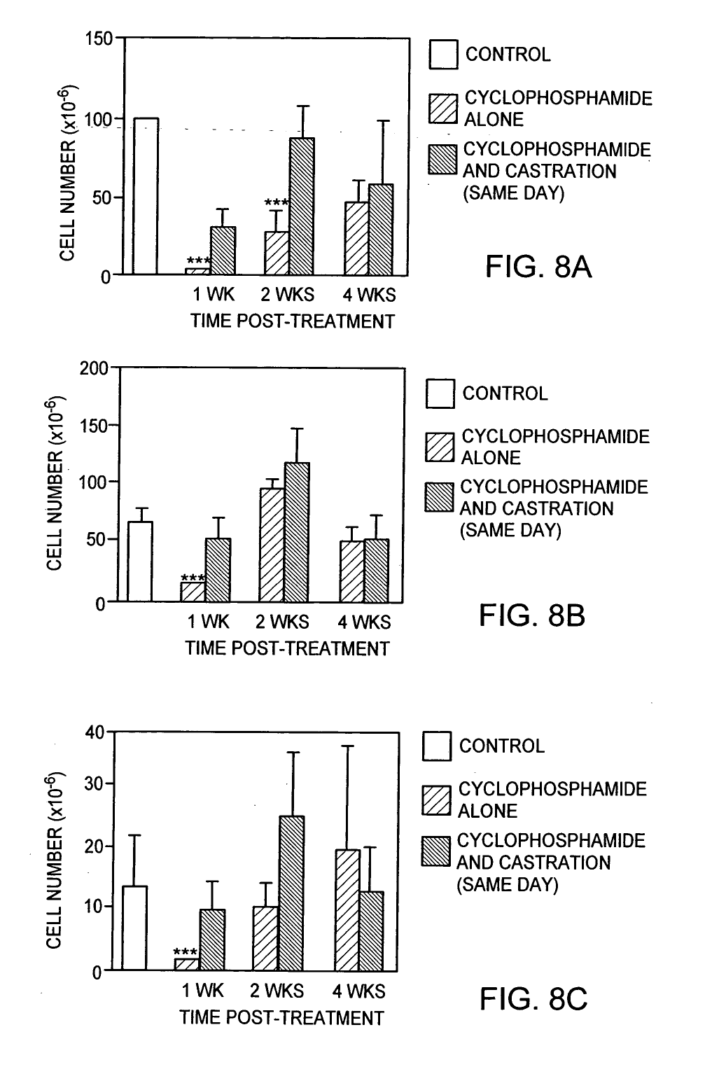

Thymus cellularity was significantly reduced in ShCx mice 1-week post-cyclophosphamide treatment compared to both control (untreated, aged-matched; p≦0.001) and Cx mice (p≦0.05) (FIG. 7A). No difference in thymus regeneration rates was observed at this time-point between mice castrated 1-week earlier or on the same day as treatment, with both groups displaying at least a doubling in the numbers of cells compared to ShCx mice (FIGS. 7A and 8A). Similarly, at 2-weeks post-cyclophosphamide treatment, both groups of Cx mice had significantly (5-6 fold) greater thymocyte numbers (p≦0.001) than the ShCx mice (FIG. 7A). In control mice there was a gradual recovery of thymocyte number over 4 weeks but this was markedly enhanced by castration—even within one week (FIG. 8A). Similarly spleen and lymph node numbers were increased in the castrate mice after one week (FIGS. 8B and 8C).

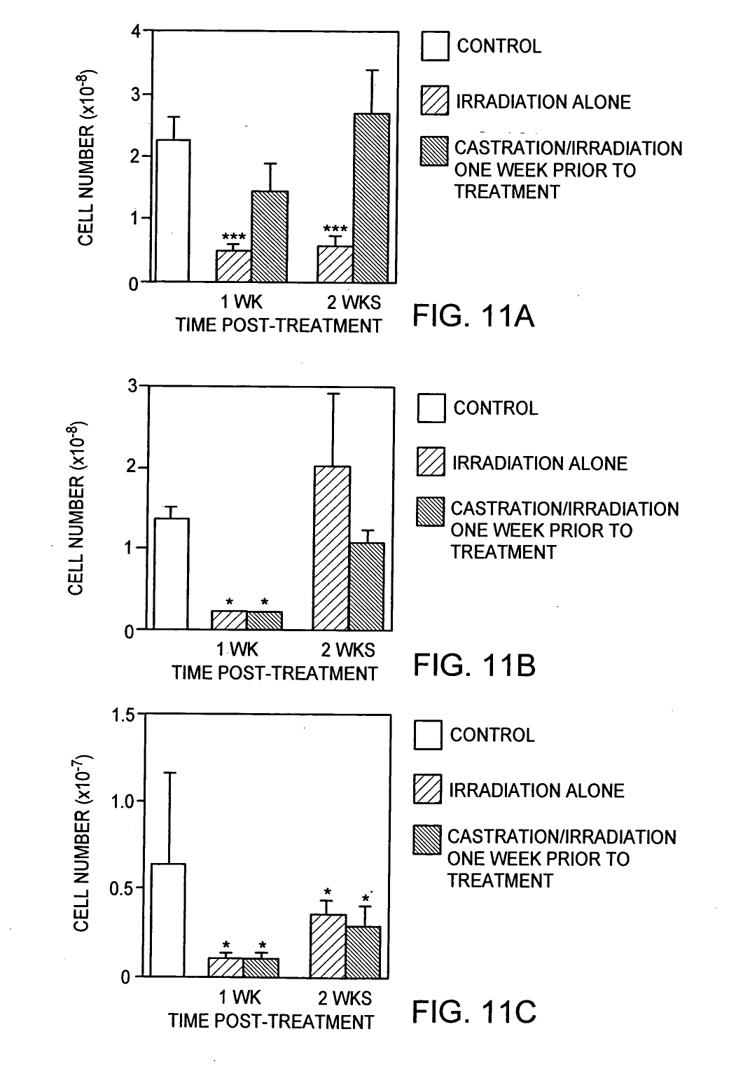

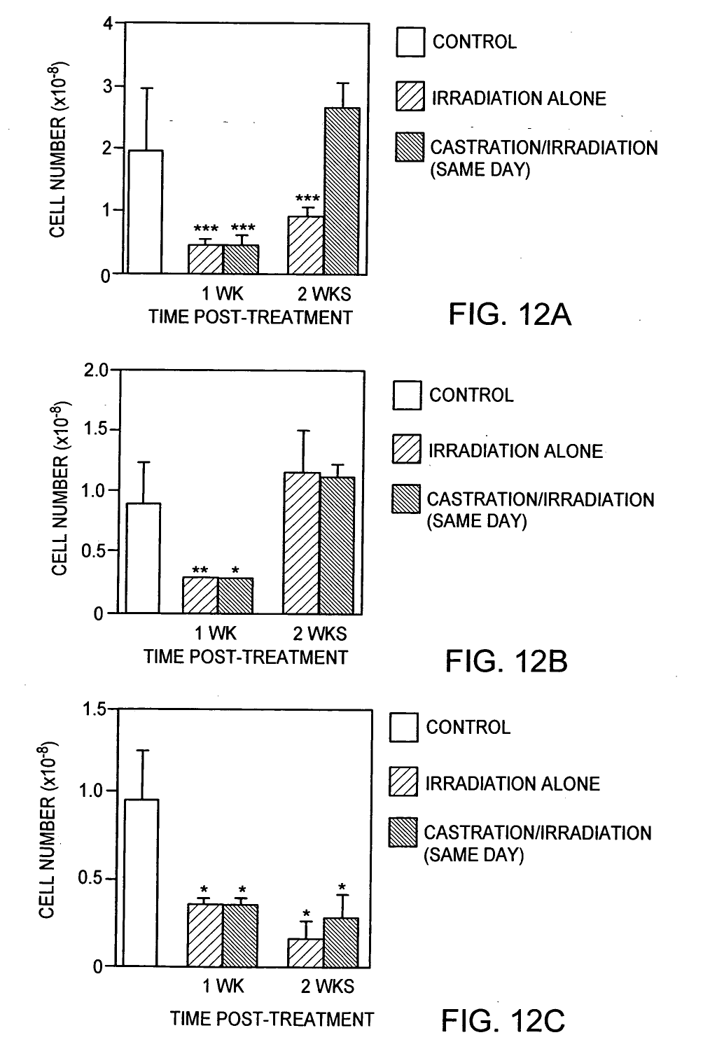

The effect of the timing of castration on thymic recovery was examined by castration one week prior to either irradiation (FIG. 11) or on the same day as irradiation (FIG. 12). When performed one week prior, castration had a more rapid impact on thymic recovery (FIG. 11A compared to FIG. 12A). By two weeks the same day castration had “caught up” with the thymic regeneration in mice castrated one week prior to treatment. In both cases there were no major effects on spleen or lymph nodes (FIGS. 11B and 11C, and FIGS. 12B and 12C) respectively.

Following irradiation treatment, both ShCx and mice castrated on the same day as treatment (SDCx) showed a significant reduction in thymus cellularity compared to control mice (p≦0.001) (FIGS. 7B and 12A) and mice castrated 1-week prior to treatment (p≦0.01) (FIG. 7B). At 2 weeks post-treatment, the castration regime played no part in the restoration of thymus cell numbers with both groups of castrated mice displaying a significant enhancement of thymus cellularity post-irradiation (PIrr) compared to ShCx mice (p≦0.001) (FIGS. 7B, 11A, and 12A). Therefore, castration significantly enhances thymus regeneration post-severe T cell depletion, and it can be performed at least 1-week prior to immune system insult.

Interestingly, thymus size appears to ‘overshoot’ the baseline of the control thymus. Indicative of rapid expansion within the thymus, the migration of these newly derived thymocytes does not yet (it takes ˜3-4 weeks for thymocytes to migrate through and out into the periphery). Therefore, although proportions within each subpopulation are equal, numbers of thymocytes are building before being released into the periphery.

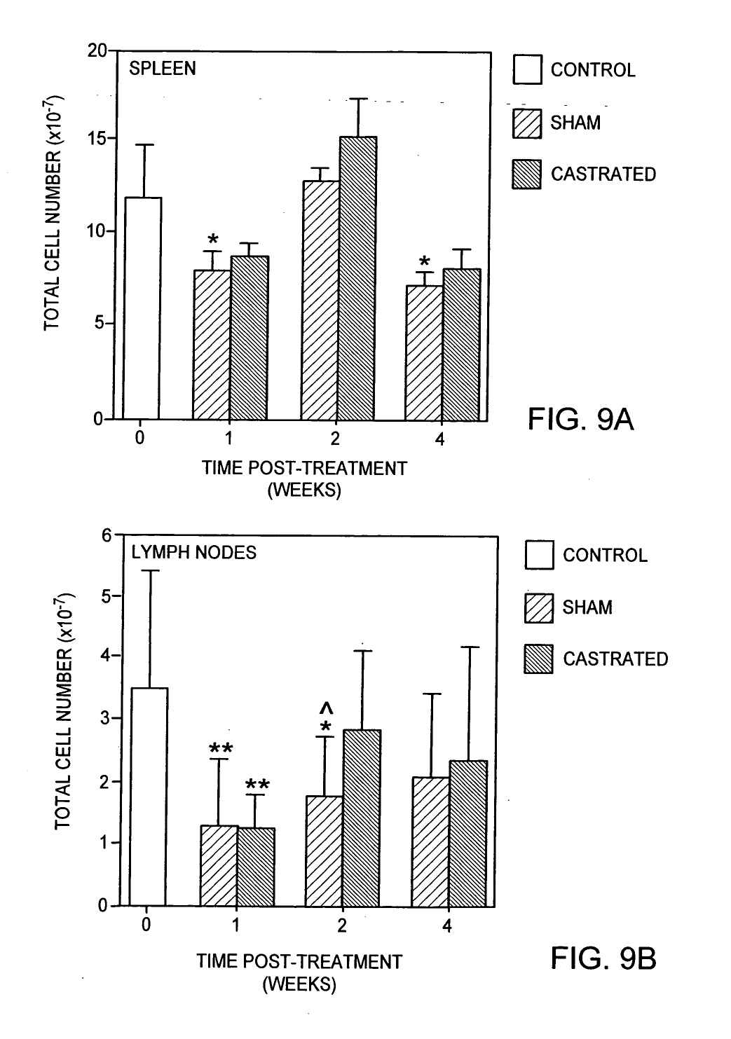

Following cyclophosphamide treatment of young mice (˜2-3 months), total lymphocyte numbers within the spleen of Cx mice, although reduced, were not significantly different from control mice throughout the time-course of analysis (FIG. 9A). However, ShCx mice showed a significant decrease in total splenocyte numbers at 1- and 4-weeks post-treatment (p≦0.05) (FIG. 9A). Within the lymph nodes, a significant decrease in cellularity was observed at 1-week post-treatment for both sham-castrated and castrated mice (p≦0.01) (FIG. 9B), possibly reflecting the influence of stress steroids. By 2-weeks post-treatment, lymph node cellularity of castrated mice was comparable to control mice however sham-castrated mice did not restore their lymph node cell numbers until 4-weeks post-treatment, with a significant (p≦0.05) reduction in cellularity compared to both control and Cx mice at 2-weeks post-treatment (FIG. 9B). These results indicate that castration may enhance the rate of recovery of total lymphocyte numbers following cyclophosphamide treatment.

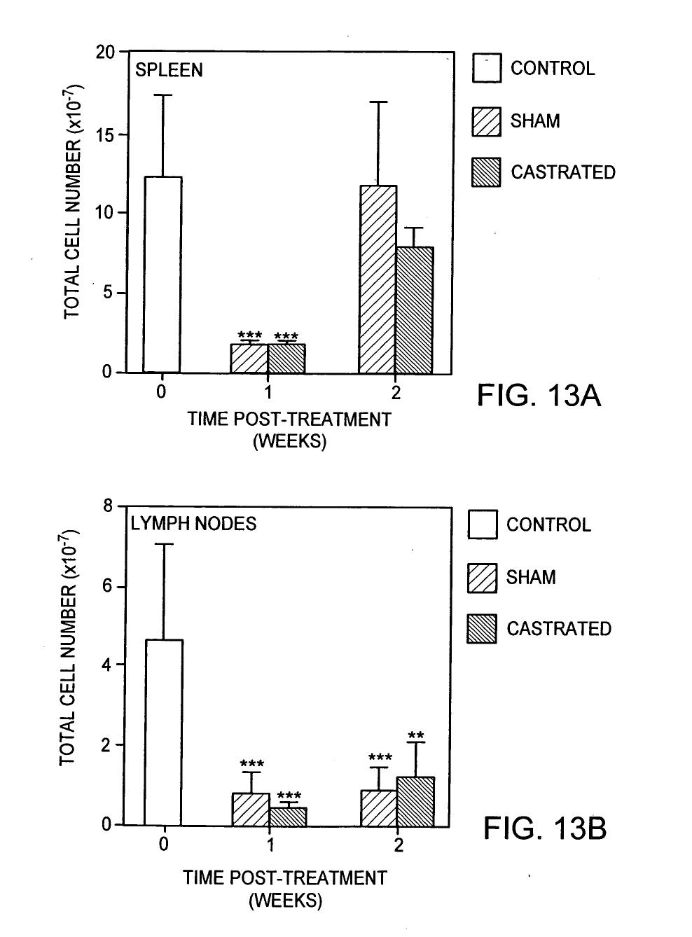

Sublethal irradiation (625 Rads) induced a profound lymphopenia such that at 1-week post-treatment, both treatment groups (Cx and ShCx), showed a significant reduction in the cellularity of both spleen and lymph nodes (p≦0.001) compared to control mice (FIGS. 13A and 13B). By 2 weeks post-irradiation, spleen cell numbers were similar to control values for both castrated and sham-castrated mice (FIG. 13A), whilst lymph node cell numbers were still significantly lower than control values (p≦0.001 for sham-castrated mice; p≦0.01 for castrated mice) (FIG. 13B). No significant difference was observed between the Cx and ShCx mice.

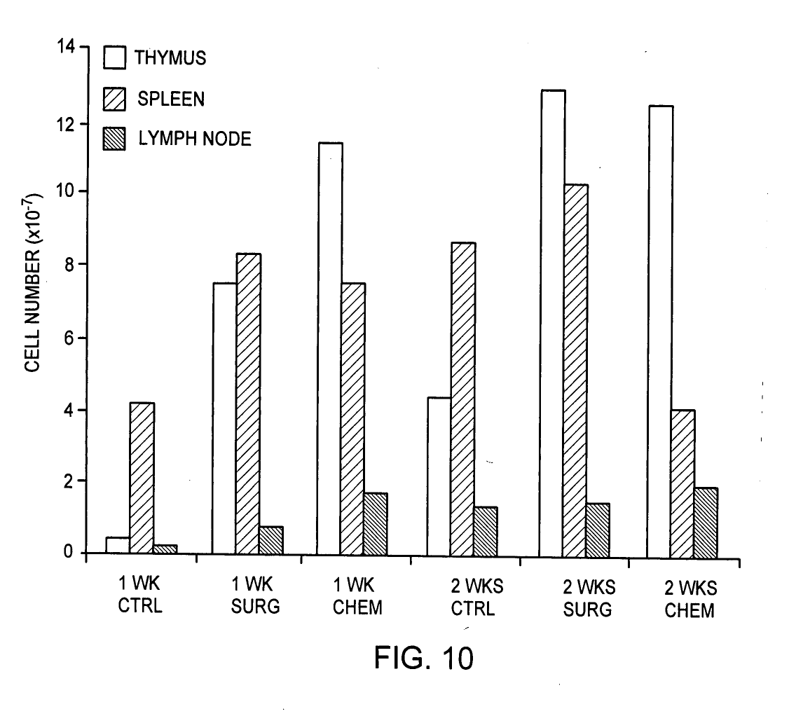

FIG. 10 illustrates the use of chemical castration compared to surgical castration in enhancement of T cell regeneration. The chemical used in this example, Deslorelin (an LHRH-A), was injected for four weeks, and showed a comparable rate of regeneration post-cyclophosphamide treatment compared to surgical castration (FIG. 10). The enhancing effects were equivalent on thymic expansion and also the recovery of spleen and lymph node (FIG. 10). The kinetics of chemical castration are slower than surgical, that is, mice take about 3 weeks longer to decrease their circulating sex steroid levels. However, chemical castration is still effective in regenerating the thymus (FIG. 10).

### Discussion

The impact of castration on thymic structure and T cell production was investigated in animal models of immunodepletion. Specifically, Example 2 examined the effect of castration on the recovery of the immune system after sublethal irradiation and cyclophosphamide treatment. These forms of immunodepletion act to inhibit DNA synthesis and therefore target rapidly dividing cells. In the thymus these cells are predominantly immature cortical thymocytes, however all subsets are effected (Fredrickson and Basch, 1994). In normal healthy aged mice, the qualitative and quantitative deviations in peripheral T cells seldom lead to pathological states. However, major problems arise following severe depletion of T cells because of the reduced capacity of the thymus for T cell regeneration. Such insults occur in HIV/AIDS, and particularly following chemotherapy and radiotherapy in cancer treatment (Mackall et al. 1995).

In both sublethally irradiated and cyclophosphamide treated mice, castration markedly enhanced thymic regeneration. Castration was carried, out on the same day as and seven days prior to immunodepletion in order to appraise the effect of the predominantly corticosteroid induced, stress response to surgical castration on thymic regeneration. Although increases in thymus cellularity and architecture were seen as early as one week after immunodepletion, the major differences were observed two weeks after castration. This was the case whether castration was performed on the same day or one week prior to immunodepletion.

Immunohistology demonstrated that in all instances, two weeks after castration the thymic architecture appeared phenotypically normal, while; that of noncastrated mice was disorganised. Pan epithelial markers demonstrated that immunodepletion caused a collapse in cortical epithelium and a general disruption of thymic architecture in the thymii of noncastrated mice. Medullary markers supported this finding. Interestingly, one of the first features of castration-induced thymic regeneration was a marked upregulation in the extracellular matrix, identified by MTS 16.

Flow cytometry analysis data illustrated a significant increase in the number of cells in all thymocyte subsets in castrated mice, corresponding with the immunofluorescence. At each time point, there was a synchronous increase in all CD4, CD8 and αβ-TCR—defined subsets following immunodepletion and castration. This is an unusual but consistent result, since T cell development is a progressive process it was expected that there would be an initial increase in precursor cells (contained within the CD4⁻CD8⁻ gate) and this may have occurred before the first time point. Moreover, since precursors represent a very small proportion of total thymocytes, a shift in their number may not have been, detectable. The effects of castration on other cells, including macrophages and granulocytes were also analysed. In general there was little alteration in macrophage and granulocyte numbers within the thymus.

In both irradiation and cyclophosphamide models of immunodepletion thymocyte numbers peaked at every two weeks and decreased four weeks after treatment. Almost immediately after irradiation or chemotherapy, thymus weight and cellularity decreased dramatically and approximately 5 days later the first phase of thymic regeneration begun. The first wave of reconstitution (days 5-14) was brought about by the proliferation of radioresistant thymocytes (predominantly double negatives) which gave rise to all thymocyte subsets (Penit and Ezine 1989). The second decrease, observed between days 16 and 22 was due to the limited proliferative ability of the radioresistant cells coupled with a decreased production of thymic precursors by the bone marrow (also effected by irradiation). The second regenerative phase was due to the replenishment of the thymus with bone marrow derived precursors (Huiskamp et al., 1983).

It is important to note that in adult mice the development from a HSC to a mature T cell takes approximately 28 days (Shortman et al., 1990). Therefore, it is not surprising that little change was seen in peripheral T cells up to four weeks after treatment. The periphery would be supported by some thyniic export, but the majority of the T cells found in the periphery up to four weeks after treatment would be expected to be proliferating cyclophosphamide or irradiation resistant clones expanding in the absence of depleted cells. Several long term changes in the periphery would be expected post-castration including, most importantly, a diversification of the TCR repertoire due to an increase in thymic export. Castration did not effect the peripheral recovery of other leukocytes, including B cells, macrophages and granulocytes.

### Example 3

### Thymic Regeneration Following Inhibition of Sex Steroids Results in Restoration of Deficient Peripheral T Cell Function

Materials and methods were as described in Examples 1 and 2.

To determine the functional consequences of thymus regeneration (e.g., whether castration can enhance the immune response, Herpes Simplex Virus (HSV) immunization was examined as it allows the study of disease progression and role of CTL (cytotoxic) T cells. Castrated mice were found to have a qualitatively and quantitatively improved responsiveness to the virus.

Mice were immunized in the footpad and the popliteal (draining) lymph node analyzed at D5 post-immunization. In addition, the footpad was removed and homogenized to determine the virus titer at particular time-points throughout the experiment. The regional (popliteal) lymph node response to HSV-1 infection (FIGS. 14-19) was examined.

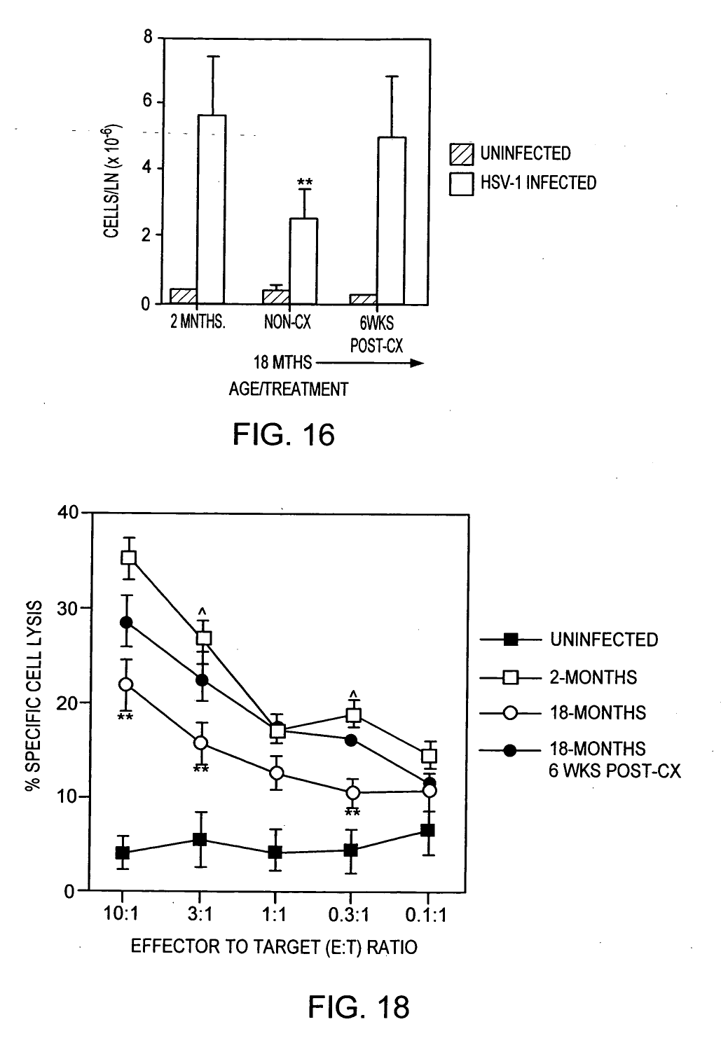

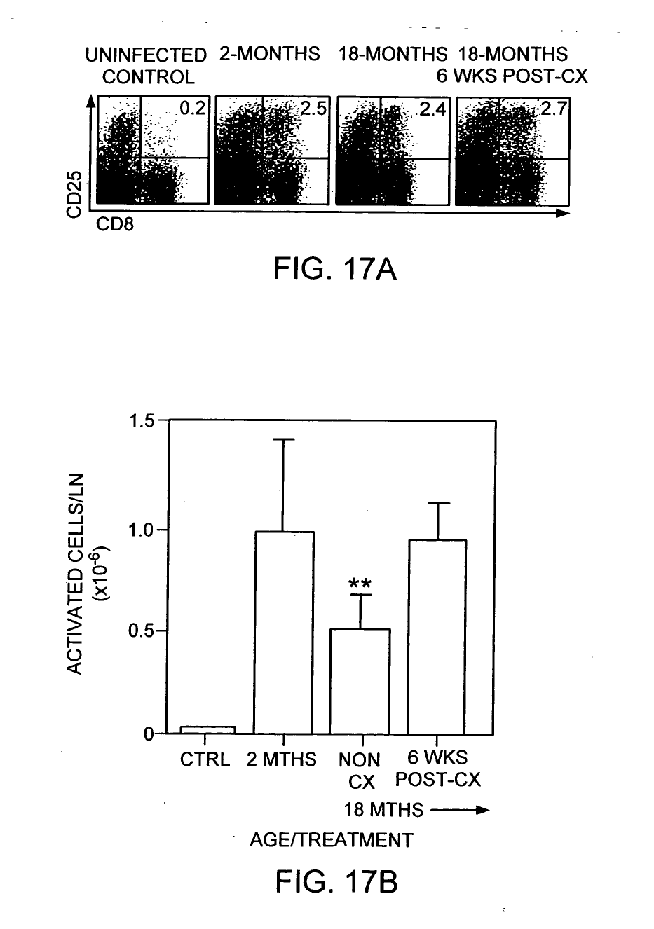

A significant decrease in lymph node cellularity was observed with age (FIGS. 14A, 14B, and 16). At D5 (i.e., 5 days) post-immunisation, the castrated mice have a significantly larger lymph node cellularity than the aged mice (FIG. 16). Although no difference in the proportion of activated (CD8⁺CD25⁺) cells was seen with age or post-castration (FIG. 17A), activated cell numbers within the lymph nodes were significantly increased with castration when compared to the aged controls (FIG. 17B). Further, activated cell numbers correlated with that found for the young adult (FIG. 17B), indicating that CTLs were being activated to a greater extent in the castrated mice, but the young adult may have an enlarged lymph node due to B cell activation. This was confirmed with a CTL assay detecting the proportion of specific lysis occurring with age and post-castration (FIG. 18). Aged mice showed a significantly reduced target cell lysis at effector:target ratios of 10:1 and 3:1 compared to young adult (2-month) mice (FIG. 18). Castration restored the ability of mice to generate specific CTL responses post-HSV infection (FIG. 18).

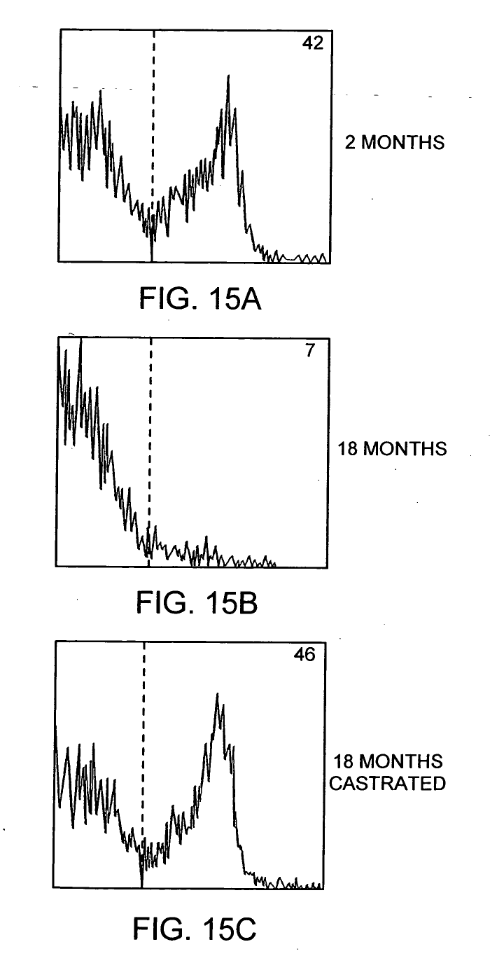

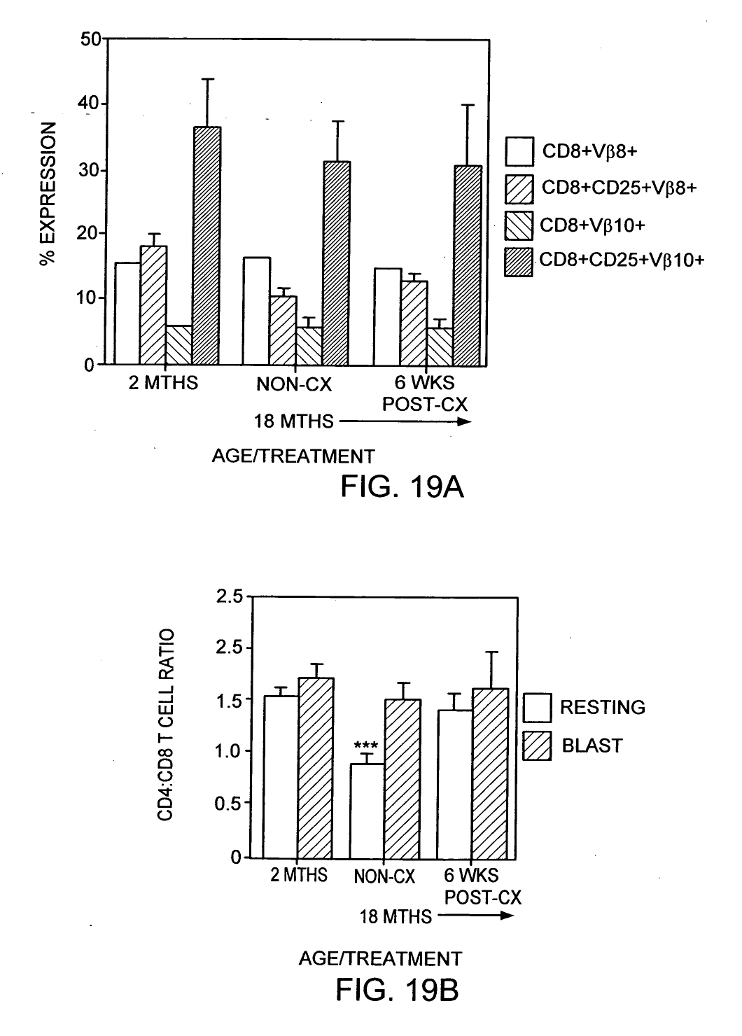

In addition, while overall expression of Vβ10 by the activated cells remained constant with age (FIG. 19A), a subgroup of aged (18-month) mice showed a diminution of this clonal response (FIGS. 15A-C). By six weeks post-castration, the total number of infiltrating lymph node cells and the number of activated CD25⁺CD8⁺ cells had increased to young adult levels (FIGS. 16 and 17B). More importantly however, castration significantly enhanced the CTL responsiveness to HSV-infected target cells, which was greatly reduced in the aged mice (FIG. 18) and restored the CD4:CD8 ratio in the lymph nodes (FIG. 19B). Indeed, a decrease in CD4+ T cells in the draining lymph nodes was seen with age compared to both young adult and castrated mice (FIG. 19B), thus illustrating the vital need for increased production of T cells from the thymus throughout life, in order to get maximal immune responsiveness.

### Inhibition of Sex Steroids Enhances Uptake of New Haemopoietic Precursor Cells Into the Thymus which Enables Chimeric Mixtures of Host an Donor Lymphoid Cells (T, B, and Dendritic Cells)

Materials and methods were as described in Examples 1 and 2.

Previous experiments have shown that microchimera formation plays an important role in organ transplant acceptance. Dendritic cells have also been shown to play an integral role in tolerance to graft antigens. Therefore, the effects of castration on thymic chimera formation and dendritic cell number was studied.

In order to assess the role of stem cell uptake in thymus regeneration, a young (3 month-old) congenic mouse model of bone marrow transplantation (BMT) was used. To do this, 3-4 month-old C57BL6/J mice were subjected to 5.5Gy irradiation twice over a 3-hour interval (lethal irradiation). One hour following the second irradiation dose, the irradiated mice were reconstituted by intravenous injection of 5×10⁶ bone marrow cells from donor 2-month old congenic C57B16/J Ly5.1⁺ mice.

For the syngeneic experiments, 4 three month old mice were used per treatment group. All controls were age matched and untreated.

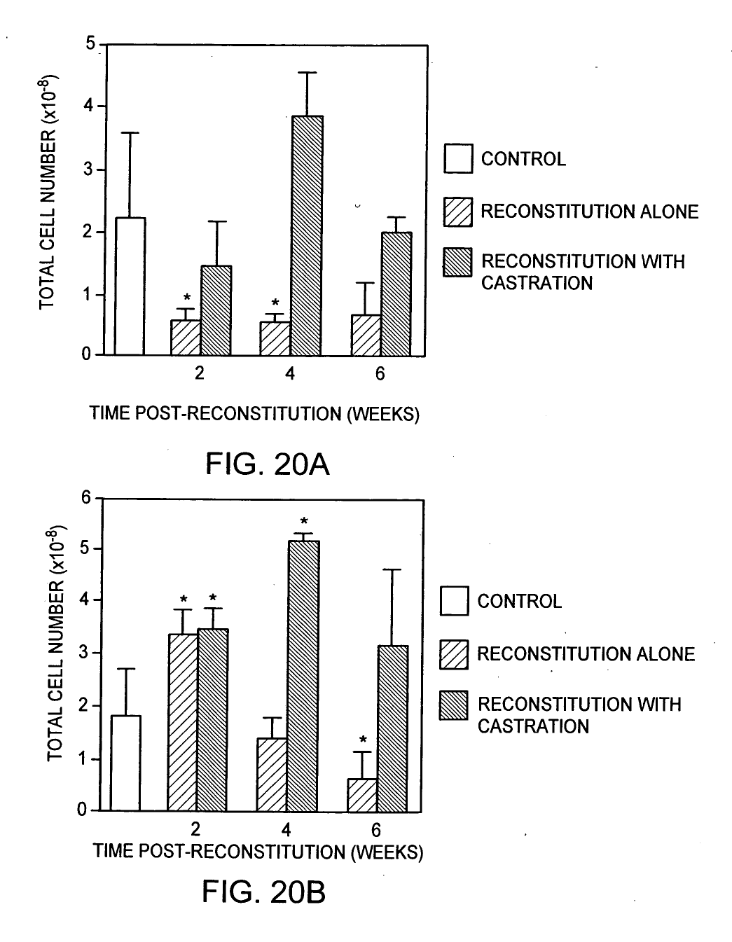

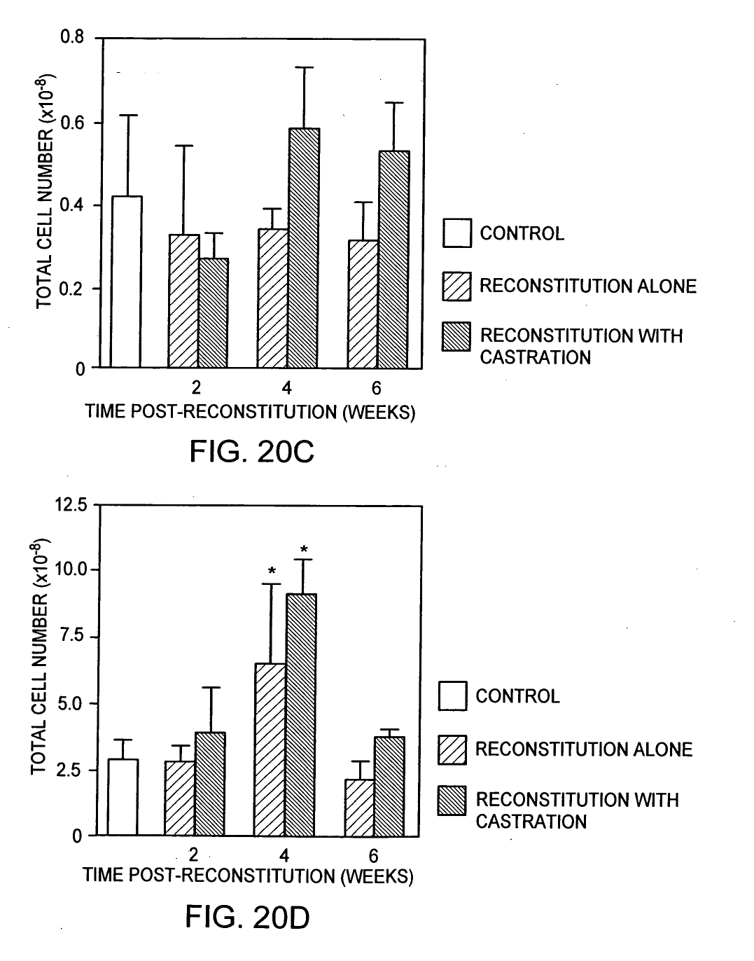

The total thymus cell numbers of castrated and noncastrated reconstituted mice were compared to untreated age matched controls and are summarized in FIG. 20A. As shown in FIG. 20A, in mice castrated 1 day prior to reconstitution, there was a significant increase (p≦0.01) in the rate of thymus regeneration compared to sham-castrated (ShCx) control mice. Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 20A). At 6 weeks, cell number remained below control levels, however, those of castrated mice was three fold higher than the noncastrated mice (p≦0.05) (FIG. 20A).

There were also significantly more cells (p≦0.05) in the BM of castrated mice 4 weeks after BMT (FIG. 20D). BM cellularity reached untreated control levels (1.5×10⁷1.5×10⁶) in the sham-castrates by 2 weeks, whereas BM cellularity was increased above control levels in castrated mice at both 2 and 4 weeks after congenic BMT (FIG. 20D). Mesenteric lymph node cell numbers were decreased 2-weeks after irradiation and reconstitution, in both castrated and noncastrated mice; however, by the 4 week time point cell numbers had reached control levels. There was no statistically significant difference in lymph node cell number between castrated and noncastrated treatment groups (FIG. 20C). Spleen cellularity reached untreated control levels (1.5×10⁷1.5×10⁶) in the sham-castrates and castrates by 2 weeks, but dropped off in the sham group over 4-6 weeks, whereas the castrated mice still had high levels of spleen cells (FIG. 20B). Again, castrated mice showed increased lymphocyte numbers at these time points (i.e., 4 and 6 weeks post-reconstitution) compared to non-castrated mice (p≦0.05) although no difference in total spleen cell number between castrated and noncastrated treatment groups was seen at 2-weeks (FIG. 20B).

Thus, in mice castrated 1 day prior to reconstitution, there was a significant increase (p≦0.01) in the rate of thymus regeneration compared to sham-castrated (ShCx) control mice (FIG. 20A). Thymus cellularity in the sham-castrated mice was below untreated control levels (7.6×10⁷±5.2×10⁶) 2 and 4 weeks after congenic BMT, while thymus cellularity of castrated mice had increased above control levels at 4-weeks post-BMT (FIG. 20A). Castrated mice had significantly increased congenic (Ly5.2) cells compared to non-castrated animals (data not shown).

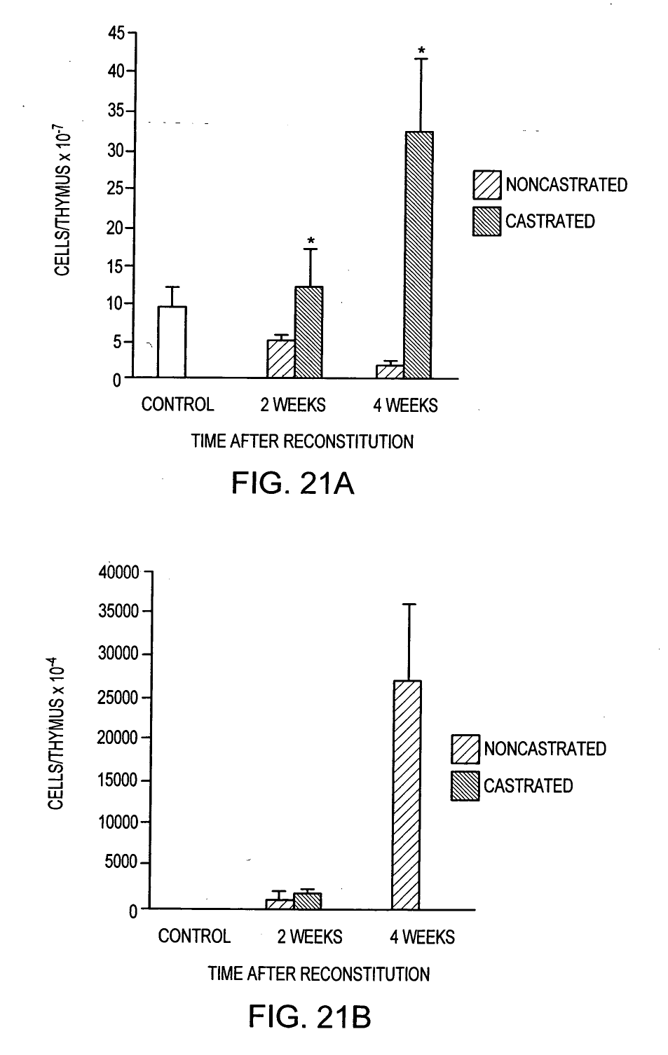

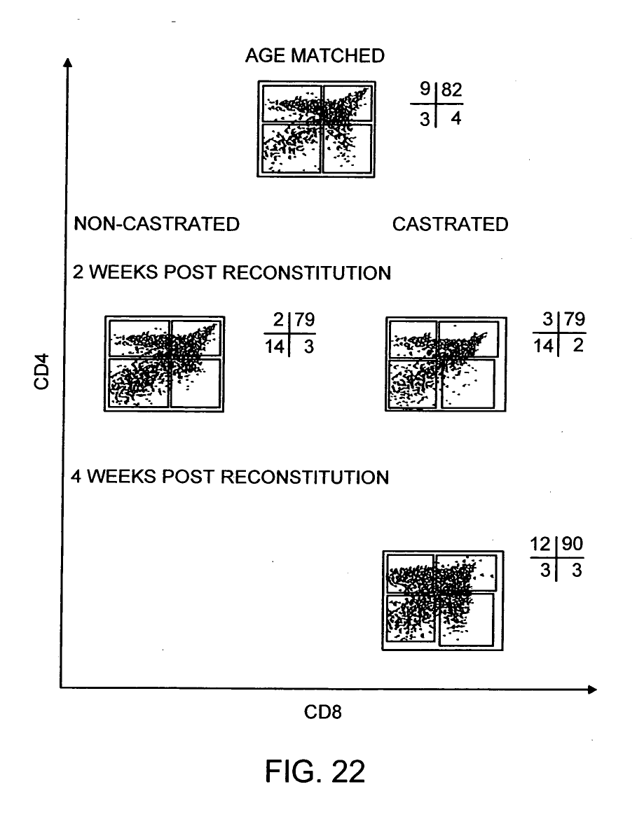

In noncastrated mice, there was a profound decrease in thymocyte number over the 4 week time period, with little or no evidence of regeneration (FIG. 21A). In the castrated group, however, by two weeks there was already extensive thymopoiesis which by four weeks had returned to control levels, being 10 fold higher than in noncastrated mice. Flow cytometeric analysis of the thymii with respect to CD45.2 (donor-derived antigen) demonstrated that no donor derived cells were detectable in the noncastrated group at 4 weeks, but remarkably, virtually all the thymocytes in the castrated mice were donor-derived at this time point (FIG. 21B). Given this extensive enhancement of thymopoiesis from donor-derived haemopoietic precursors, it was important to determine whether T cell differentiation had proceeded normally. CD4, CD8 and TCR defined subsets were analysed by flow cytometry. There were no proportional differences in thymocytes subset proportions 2 weeks after reconstitution (FIG. 22). This observation was not possible at 4 weeks, because the noncastrated mice were not reconstituted with donor-derived cells. However, at this time point the thymocyte proportions in castrated mice appear normal.

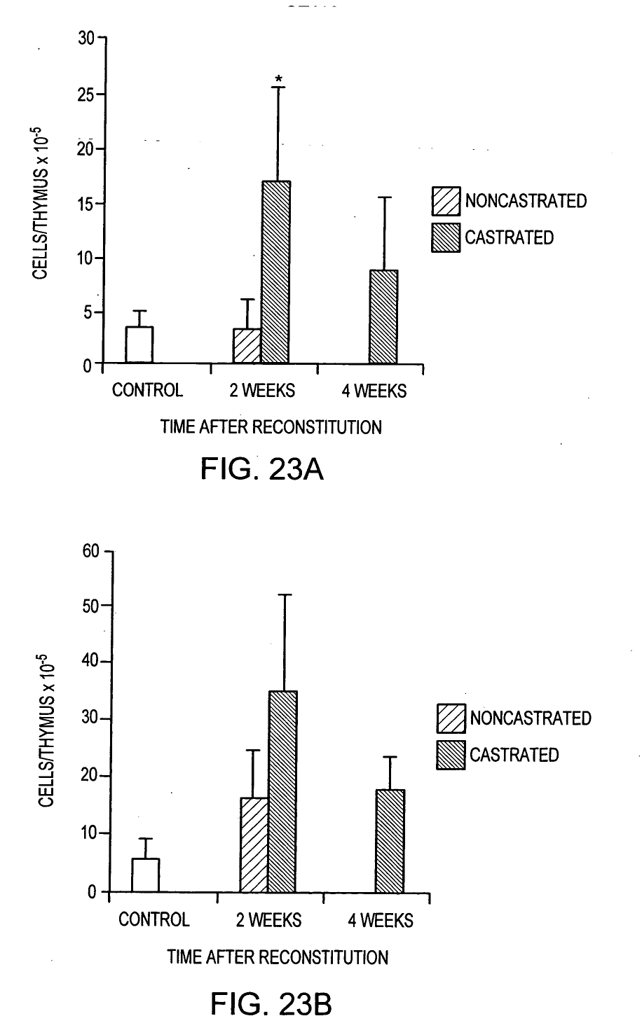

Two weeks after foetal liver reconstitution there were significantly more donor-derived, myeloid dendritic cells (defined as CD45.2+Mac 1+CD11C+) in castrated mice than noncastrated mice, the difference was 4-fold (p≦0.05). Four weeks after treatment the number of donor-derived myeloid dendritic cells remained above the control in castrated mice (FIG. 23A). Two weeks after foetal liver reconstitution the number of donor derived lymphoid dendritic cells (defined as CD45.2+Mac1-CD11C+) in the thymus of castrated mice was double that found in noncastrated mice. Four weeks after treatment the number of donor-derived lymphoid dendritic cells remained above the control in castrated mice (FIG. 23B).

Immunofluorescent staining for CD11C, epithelium (antikeratin) and CD45.2 (donor-derived marker) localized dendritic cells to the corticomedullary junction and medullary areas of thymii 4 weeks after reconstitution and castration. Using colocalization software, donor-derivation of these cells was confirmed (data not shown). This was supported by flow cytometry data suggesting that 4 weeks after reconstitution approximately 85% of cells in the thymus are donor derived.

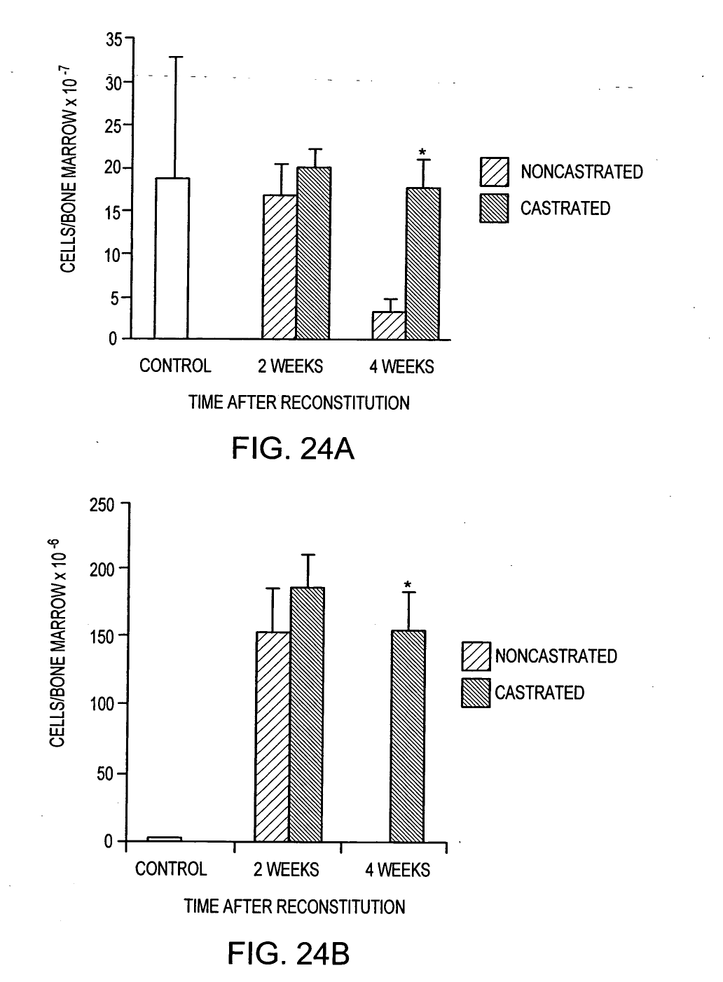

Cell numbers in the bone marrow of castrated and noncastrated reconstituted mice were compared to those of untreated age matched controls and are summarised in FIG. 24A. Bone marrow cell numbers were normal two and four weeks after reconstitution in castrated mice. Those of noncastrated mice were normal at two weeks but dramatically decreased at four weeks (p<0.05). Although, at this time point the noncastrated mice did not reconstitute with donor-derived cells.

Flow cytometeric analysis of the bone marrow with respect to CD45.2 (donor-derived antigen) established that no donor derived cells were detectable in the bone marrow of noncastrated mice 4 weeks after reconstitution, however, almost all the cells in the castrated mice were donor- derived at this time point (FIG. 24B).

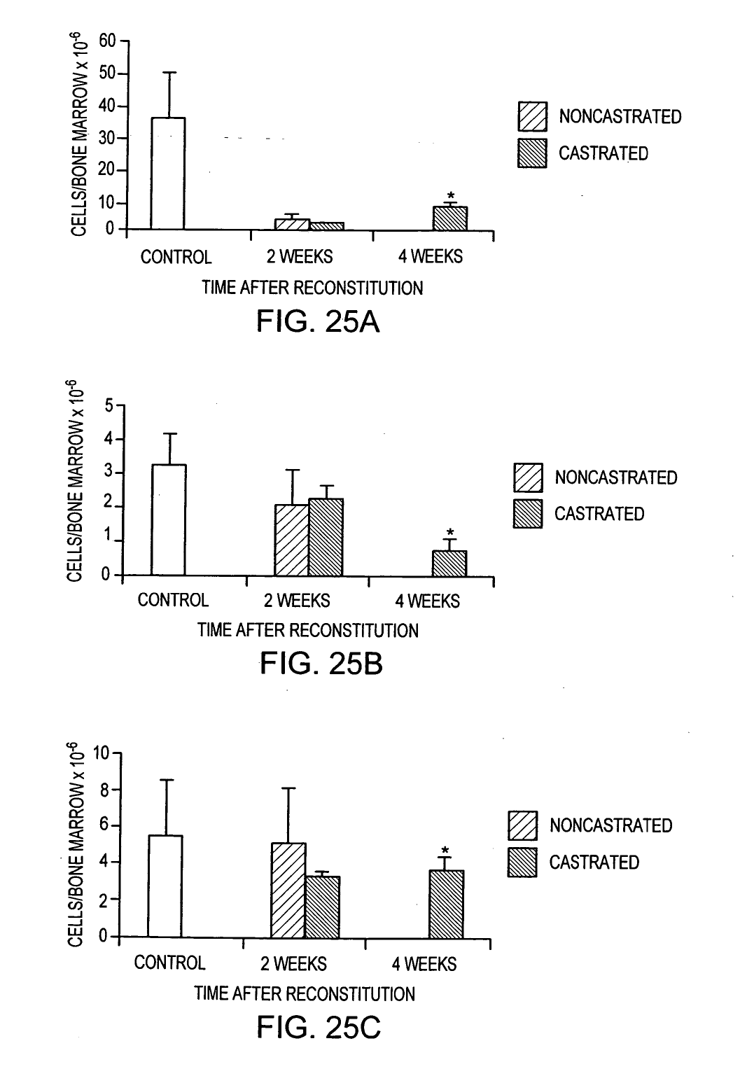

Two weeks after reconstitution the donor-derived T cell numbers of both castrated and noncastrated mice were markedly lower than those seen in the control mice (p<0.05). At 4 weeks there were no donor-derived T cells in the bone marrow of noncastrated mice and T cell number remained below control levels in castrated mice (FIG. 25A).

Donor-derived, myeloid and lymphoid dendritic cells were found at control levels in the bone marrow of noncastrated and castrated mice 2 weeks after reconstitution. Four weeks after treatment numbers decreased further in castrated mice and no donor-derived cells were seen in the noncastrated group (FIG. 25B).

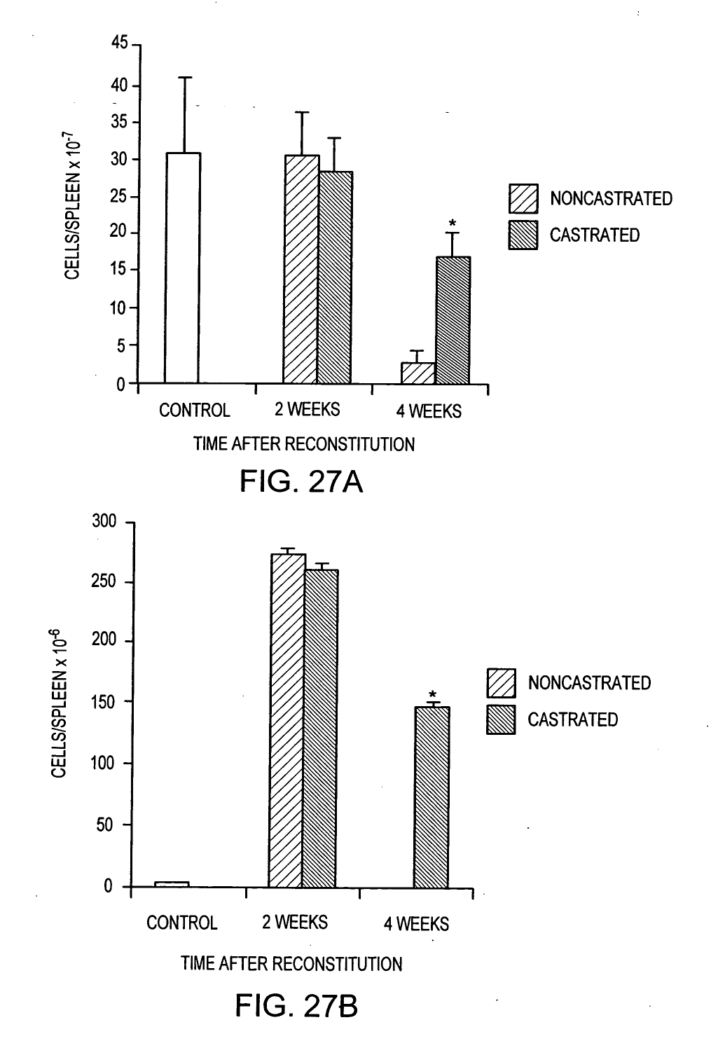

Spleen cell numbers of castrated and noncastrated reconstituted mice were compared to untreated age matched controls and the results are summarised in FIG. 27A. Two weeks after treatment, spleen cell numbers of both castrated and noncastrated mice were approximately 50% that of the control. By four weeks, numbers in castrated mice were approaching normal levels, however, those of noncastrated mice remained decreased. Analysis of CD45.2 (donor-derived) flow cytometry data demonstrated that there was no significant difference in the number of donor derived cells of castrated and noncastrated mice, 2 weeks after reconstitution (FIG. 27B). No donor derived cells were detectable in the spleens of noncastrated mice at 4 weeks, however, almost all the spleen cells in the castrated mice were donor derived.

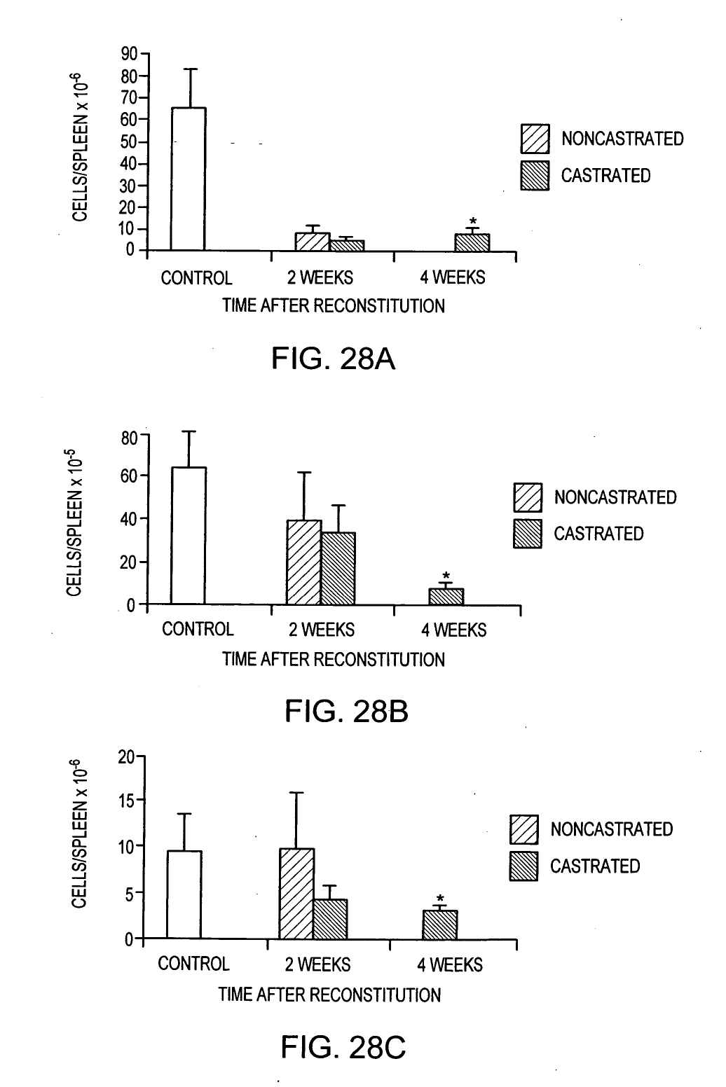

Two and four weeks after reconstitution there was a marked decrease in T cell number in both castrated and noncastrated mice (p<0.05) (FIG. 28A). Two weeks after foetal liver reconstitution donor-derived myeloid and lymphoid dendritic cells (FIGS. 28A and 28B, respectively) were found at control levels in noncastrated and castrated mice. At 4 weeks no donor derived dendritic cells were detectable in the spleens of noncastrated mice and numbers remained decreased in castrated mice.

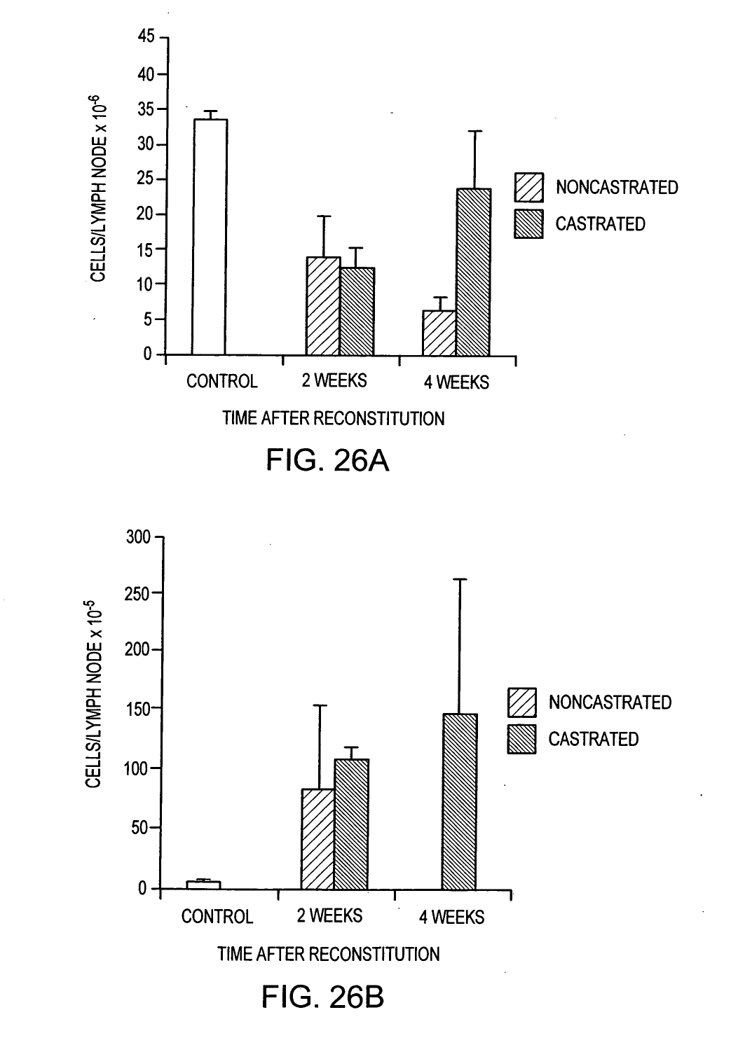

Lymph node cell numbers of castrated and noncastrated, reconstituted mice were compared to those of untreated age matched controls and are summarised in FIG. 26A. Two weeks after reconstitution cell numbers were at control levels in both castrated and noncastrated mice. Four weeks after reconstitution, cell numbers in castrated mice remained at control levels but those of noncastrated mice decreased significantly (FIG. 26B). Flow cytometry analysis with respect to CD45.2 suggested that there was no significant difference in the number of donor-derived cells, in castrated and noncastrated mice, 2 weeks after reconstitution (FIG. 26B). No donor derived cells were detectable in noncastrated mice 4 weeks after reconstitution. However, virtually all lymph node cells in the castrated mice were donor-derived at the same time point.

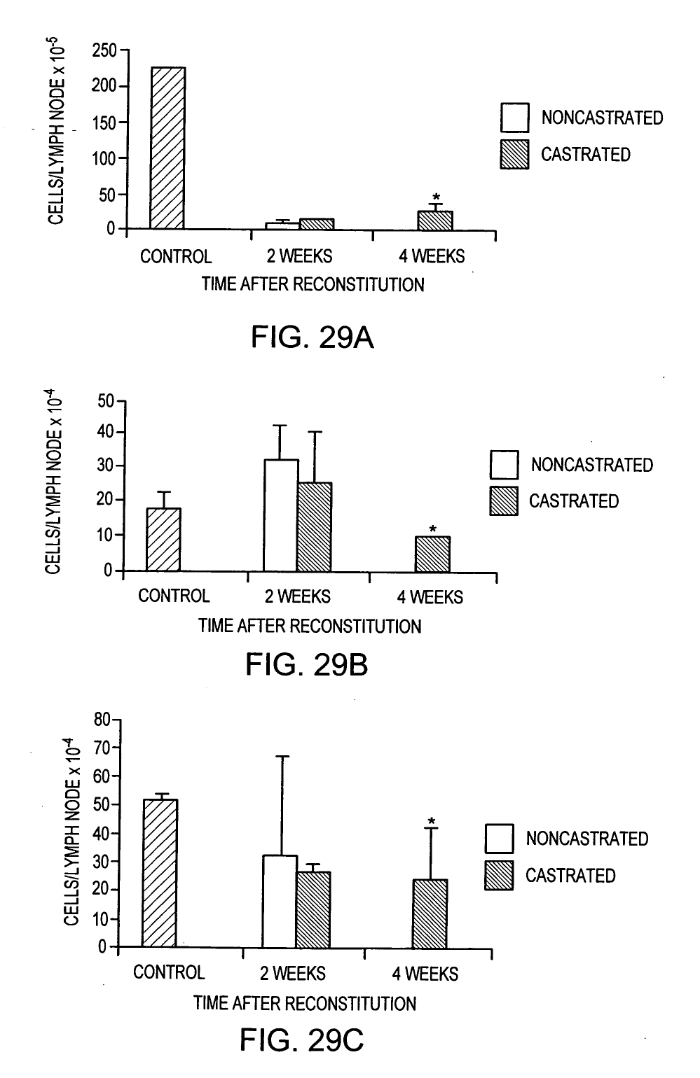

Two and four weeks after reconstitution donor-derived T cell numbers in both castrated and noncastrated mice were lower than control levels. At 4 weeks the numbers remained low in castrated mice and there were no donor-derived T cells in the lymph nodes of noncastrated mice (FIG. 29). Two weeks after foetal liver reconstitution donor-derived, myeloid and lymphoid dendritic cells were found at control levels in noncastrated and castrated mice (FIGS. 29A and 29B, respectively). Four weeks after treatment the number of donor-derived myeloid dendritic cells fell below the control, however, lymphoid dendritic cell number remained unchanged Thus, castrated mice had significantly increased congenic (Ly5.2) cells compared to non-castrated animals. The observed increase in thymus cellularity of castrated mice was predominantly due to increased numbers of donor-derived thymocytes (FIGS. 21 and 23), which correlated with increased numbers of HSC (Lin⁻c-kit⁺sca-1⁺) in the bone marrow of the castrated mice. In addition, castration enhanced generation of B cell precursors and B cells in the marrow following BMT, although this did not correspond with an increase in peripheral B cell numbers at the time-points. Thus, thymic regeneration most likely occurs through synergistic effects on stem cell content in the marrow and their uptake and/or promotion of intrathymic proliferation and differentiation. Importantly, intrathymic analysis demonstrated a significant increase (p≦0.05) in production of donor-derived DC in Cx mice compared to ShCx mice (FIG. 23B) concentrated at the corticomedullary junction as is normal for host DC (data not shown). In all cases of thymic reconstitution, thymic structure and cellularity was identical to that of young mice (data not shown).

These HSC transplants (BM or fetal liver) clearly showed the development of host DC's (and T cells) in the regenerating thymus in a manner identical to that which normally occurs in the thymus. There was also a reconstitution of the spleen and lymph node in the transplanted mice which was much more profound in the castrated mice at 4 weeks (see, e.g., FIGS. 24, 26, 27, 28, and 29). Since the host HSC clearly enter the patient thymus and create DC which localize in the same regions as host DC in the normal thymus (confirmed by immunohistology; data not shown) it is highly likely that such chimeric thymii will generate T cells tolerant to the donor (by negative selection occurring in donor-reactive T cells after contacting donor DC). This establishes a clear approach to inducing transplantation tolerance because it is long lasting (because the donor HSC are self-renewing) and not requiring prolonged immunosuppression, being due to the actual death of potentially reactive clones.

In a parallel set of experiments, 3 month old, young adults, C57/BL6 mice were castrated or sham-castrated 1 day prior to BMT. For congenic BMT, the mice were subjected to 800RADS TBI and IV injected with 5×10⁶ Ly5.1⁺ BM cells. Mice were killed 2 and 4 weeks later and the BM, thymus and spleen were analyzed for immune reconstitution. Donor/Host origin was determined with anti-CD45.1 antibody, which only reacts with leukocytes of donor origin.

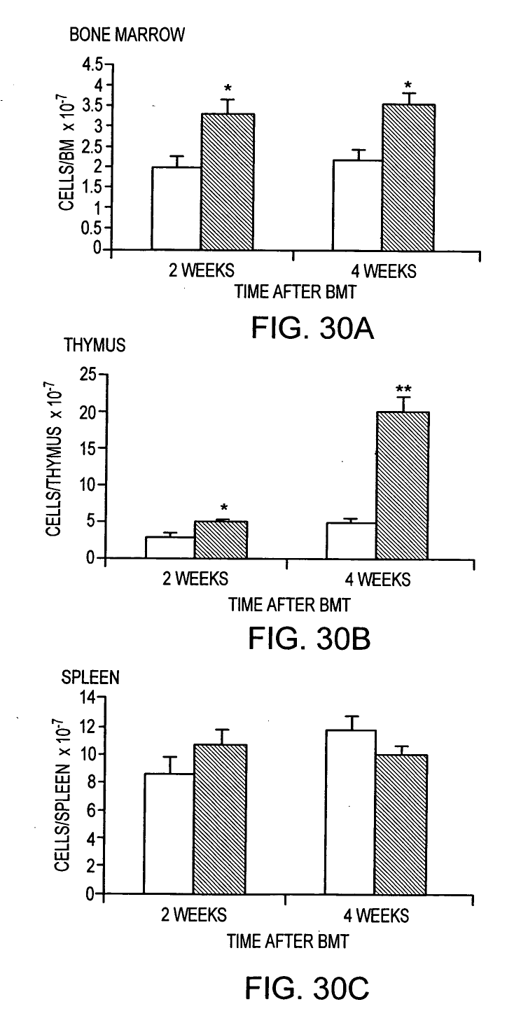

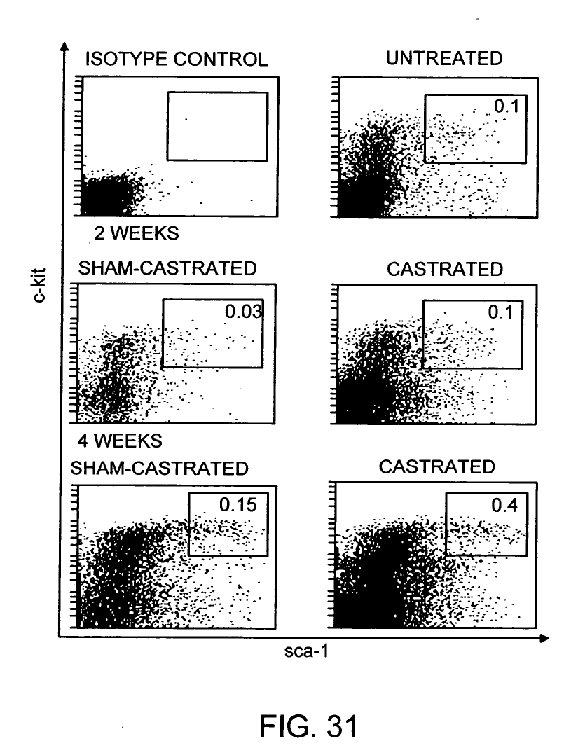

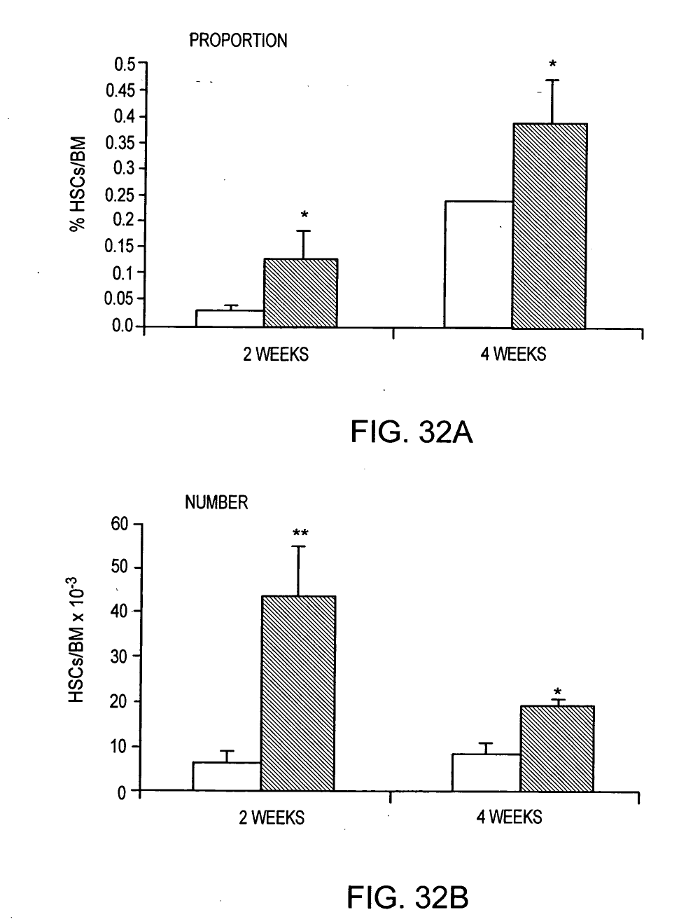

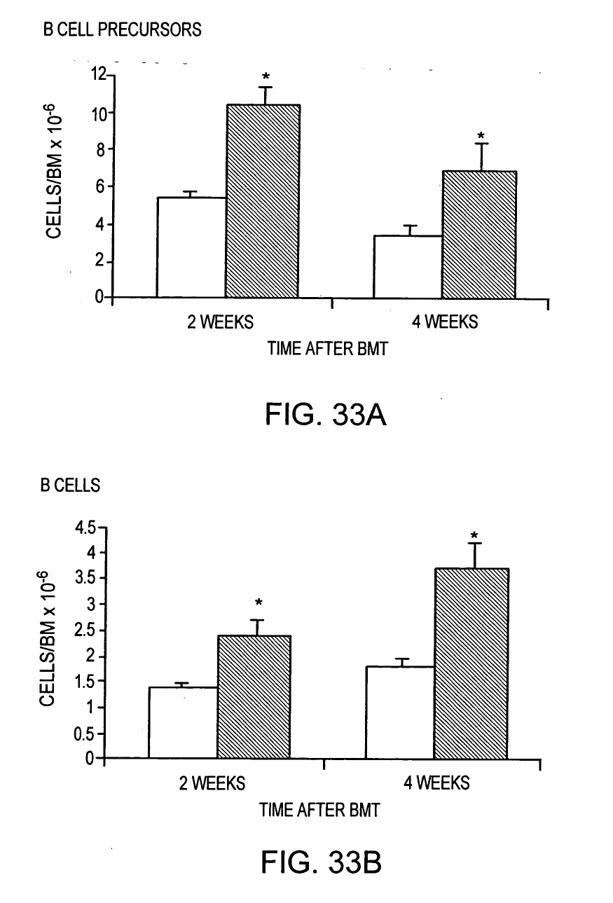

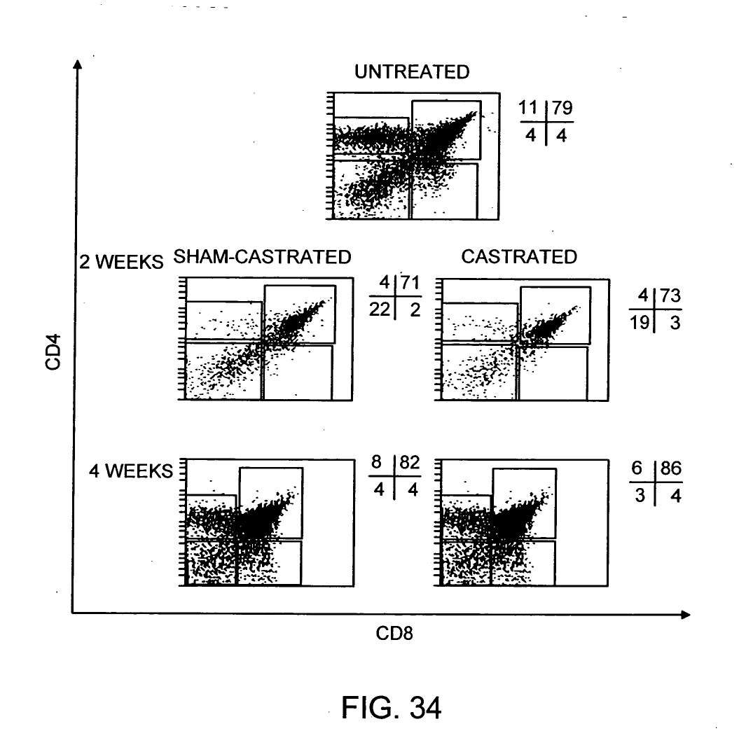

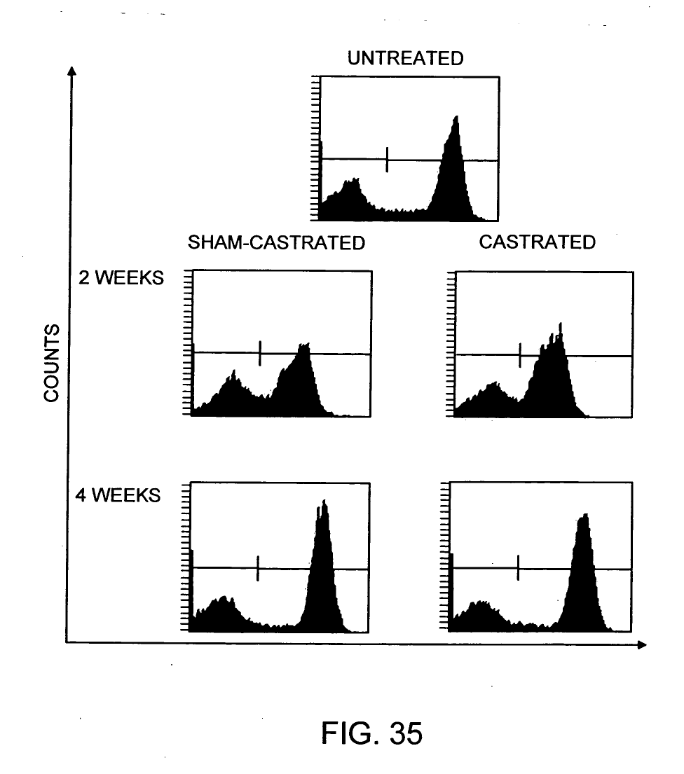

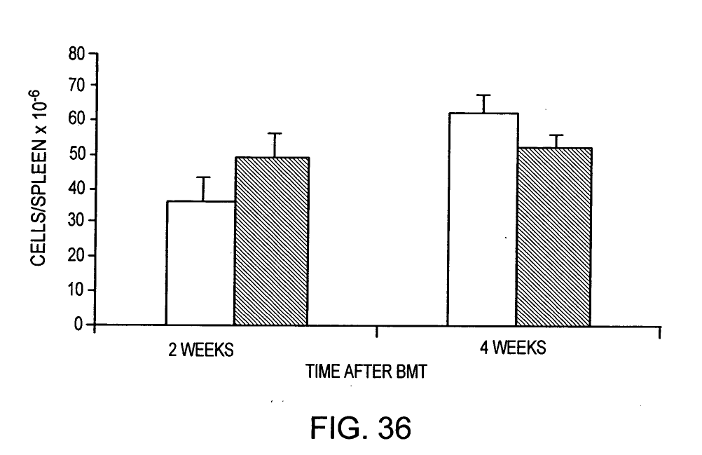

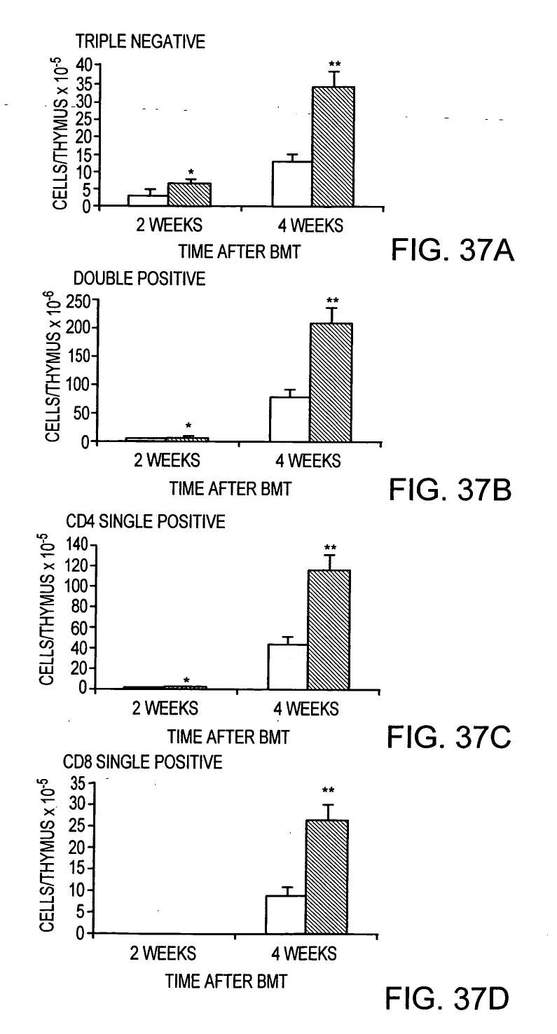

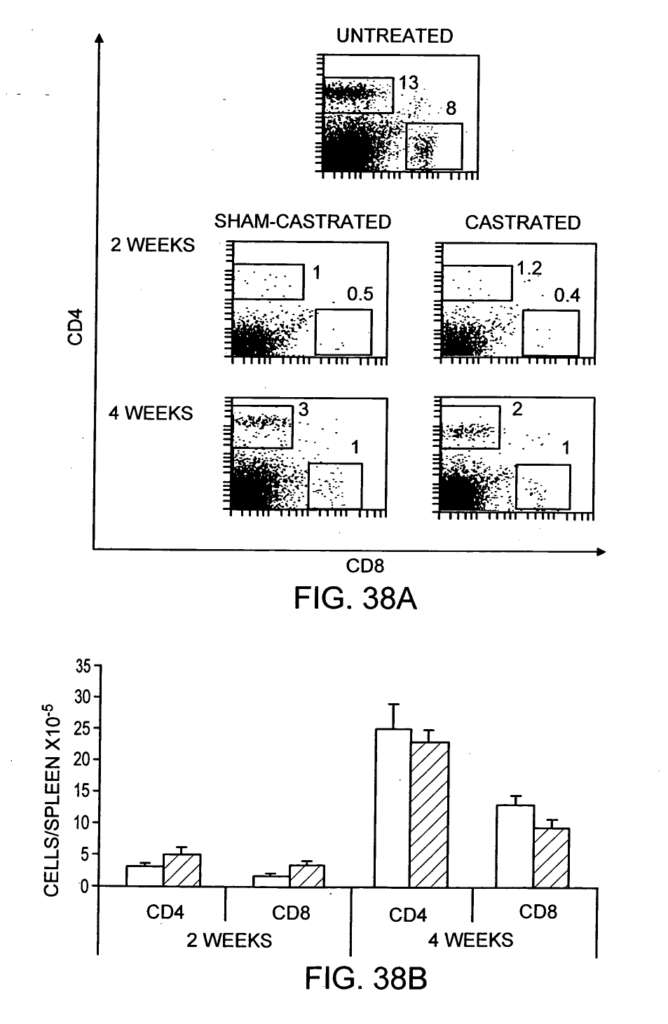

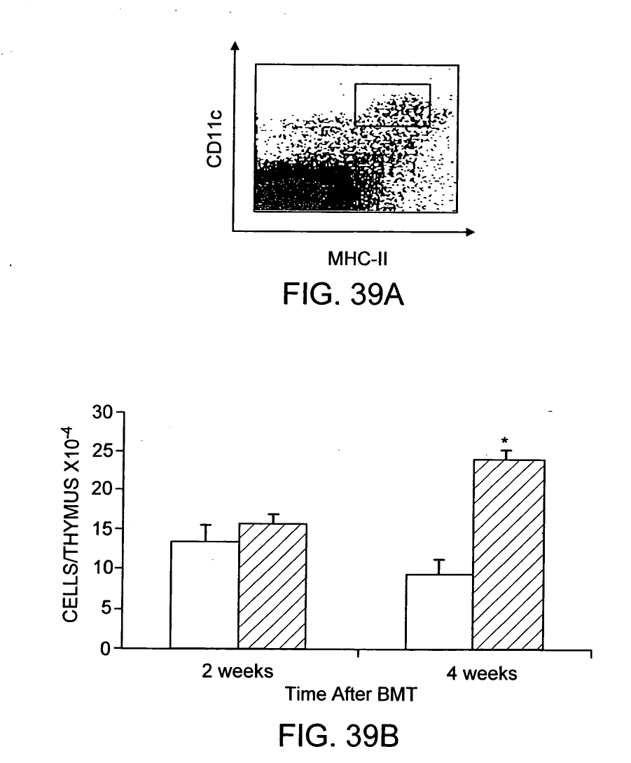

The results from this parallel set of experiments are shown in FIGS. 30-39.

### Discussion

Example 4 shows the influence of castration on syngeneic and congenic bone marrow transplantation. Starzl et al. (1992) reported that microchimeras evident in lymphoid and nonlymphoid tissue were a good prognostic indicator for allograft transplantation. That is it was postulated that they were necessary for the induction of tolerance to the graft (Starzl et al., 1992). Donor-derived dendritic cells were present in these chimeras and were thought to play an integral role in the avoidance of graft rejection (Thomson and Lu, 1999). Dendritic cells are known to be key players in the negative selection processes of thymus and if donor-derived dendritic cells were present in the recipient thymus, graft reactive T cells may be deleted.

In order to determine if castration would enable increased chimera formation, a study was performed using syngeneic foetal liver transplantation. The results showed an enhanced regeneration of thymii of castrated mice. These trends were again seen when the experiments were repeated using congenic (Ly5) mice. Due to the presence of congenic markers, it was possible to assess the chimeric status of the mice. As early as two weeks after foetal liver reconstitution there were donor-derived dendritic cells detectable in the thymus, the number in castrated mice being four-fold higher than that in noncastrated mice. Four weeks after reconstitution the noncastrated mice did not appear to be reconstituted with donor derived cells, suggesting that castration may in fact increase the probability of chimera formation. Given that castration not only increases thymic regeneration after lethal irradiation and foetal liver reconstitution and that it also increases the number of donor-derived dendritic cells in the thymus, along-side stem cell transplantation this approach increases the probability of graft acceptance.

### Example

### T Cell Depletion

In order to prevent interference with the graft by the existing T cells in the potential graft recipient patient, the patient underwent T cell depletion. One standard procedure for this step is as follows. The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment did not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus.

The prevention of T cell reactivity may also be combined with inhibitors of second level signals such as interleukins, accessory molecules (e.g., antibodies blocking, e.g., CD28), signal transduction molecules or cell adhesion molecules to enhance the T cell ablation. The thymic reconstitution phase would be linked to injection of donor HSC (obtained at the same time as the organ or tissue in question either from blood—pre-mobilized from the blood with G-CSF (2 intradermal injections/day for 3 days) or collected directly from the bone marrow of the donor. The enhanced levels of circulating HSC would promote uptake by the thymus (activated by the absence of sex steroids and/or the elevated levels of GnRH). These donor HSC would develop into intrathymic dendritic cells and cause deletion of any newly formed T cells which by chance would be “donor-reactive”. This would establish central tolerance to the donor cells and tissues and thereby prevent or greatly minimize any rejection by the host. The development of a new repertoire of T cells would also overcome the immunodeficiency caused by the T cell-depletion regime.

The depletion of peripheral T cells minimizes the risk of graft rejection because it depletes non-specifically all T cells including those potentially reactive against a foreign donor. Simultaneously, however, because of the lack of T cells the procedure induces a state of generalized immunodeficiency which means that the patient is highly susceptible to infection, particularly viral infection. Even B cell responses will not function normally in the absence of appropriate T cell help.

### Example 6

### Sex Steroid Ablation Therapy

The patient was given sex steroid ablation therapy in the form of delivery of an LHRH agonist. This was given in the form of either Leucrin (depot injection; 22.5 mg) or Zoladex (implant; 10.8 mg), either one as a single dose effective for 3 months. This was effective in reducing sex steroid levels sufficiently to reactivate the thymus. In other words, the serum levels of sex steroids were undetectable (castrate; <0.5 ng/ml blood). In some cases it is also necessary to deliver a suppresser of adrenal gland production of sex steroids. Cosudex (5 mg/day) as one tablet per day may be delivered for the duration of the sex steroid ablation therapy. Adrenal gland production of sex steroids makes up around 10-15% of a human's steroids.

Reduction of sex steroids in the blood to minimal values took about 1-3 weeks; concordant with this was the reactivation of the thymus. In some cases it is necessary to extend the treatment to a second 3 month injection/implant. The thymic expansion may be increased by simultaneous enhancement of blood HSC either as an allogeneic donor (in the case of grafts of foreign tissue) or autologous HSC (by injecting the host with G-CSF to mobilize these HSC from the bone marrow to the thymus.

### Example 7

### Alternative Delivery Method

In place of the 3 month depot or implant administration of the LHRH agonist, alternative methods can be used. In one example the patient's skin may be irradiated by a laser such as an Er:YAG laser, to ablate or alter the skin so as to reduce the impeding effect of the stratum corneum.

Laser Ablation or Alteration. An infrared laser radiation pulse was formed using a solid state, pulsed, Er:YAG laser consisting of two flat resonator mirrors, an Er:YAG crystal as an active medium, a power supply, and a means of focusing the laser beam. The wavelength of the laser beam was 2.94 microns. Single pulses were used.

The operating parameters were as follows: The energy per pulse was 40, 80 or 120 mJ, with the size of the beam at the focal point being 2 mm, creating an energy fluence of 1.27, 2.55 or 3.82 J/cm². The pulse temporal width was 300 μs, creating an energy fluence rate of 0.42, 0.85 or 1.27×10⁴ W/cm².

Subsequently, an amount of LHRH agonist is applied to the skin and spread over the irradiation site. The LHRH agonist may be in the form of an ointment so that it remains on the site of irradiation. Optionally, an occlusive patch is placed over the agonist in order to keep it in place over the irradiation site.

Optionally a beam splitter is employed to split the laser beam and create multiple sites of ablation or alteration. This provides a faster flow of LHRH agonist through the skin into the blood stream. The number of sites can be predetermined to allow for maintenance of the agonist within the patient's system for the requisite approximately 30 days.

Pressure Wave. A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate the laser beam, which hits the target material and generates a single impulse transient. The black polystyrene target completely absorbs the laser radiation so that the skin is exposed only to the impulse transient, and not laser radiation. No pain is produced from this procedure. The procedure can be repeated daily, or as often as required, to maintain the circulating blood levels of the agonist.

### Example 8

### Optional Administration of Donor HSC

Where practical, the level of hematopoietic stem cells (HSC) in the donor blood is enhanced by injecting into the donor granulocyte-colony stimulating factor (G-CSF) at 10 μg/kg for 2-5 days prior to cell collection (e.g., one or two injections of 10 μg/kg per day for each of 2-5 days). CD34⁺ donor cells are purified from the donor blood or bone marrow, such as by using a flow cytometer or immunomagnetic beading. Antibodies that specifically bind to human CD34 are commercially available (from, e.g., Research Diagnostics Inc., Flanders, N.J.). Donor-derived HSC are identified by flow cytometry as being CD34⁺. These CD34+ HSC may also be expanded by in vitro culture using feeder cells (e.g., fibroblasts), growth factors such as stem cell factor (SCF), and LIF to prevent differentiation into specific cell types. At approximately 3-4 weeks post LHRH agonist delivery (i.e., just before or at the time the thymus begins to regenerate) the patient is injected with the donor HSC, optimally at a dose of about 2-4×10⁶ cells/kg. G-CSF may also be injected into the recipient to assist in expansion of the donor HSC. If this timing schedule is not possible because of the critical nature of clinical condition, the HSC could be administered at the same time as the GnRH. It may be necessary to give a second dose of HSC 2-3 weeks later to assist in the thymic regrowth and the development of donor DC (particularly in the thymus). Once the HSC have engraftment (i.e., have incorporated into the bone marrow and thymus), the effects should be permanent since the HSC are self-renewing.

The reactivated thymus takes up the purified HSC and converts them into donor-type T cells and dendritic cells, while converting the recipient's HSC into recipient-type T cells and dendritic cells. By inducing deletion by cell death, or by inducing tolerance through immunoregulatory cells, the donor and host dendritic cells will tolerize any new T cells that are potentially reactive with donor or recipient

In one embodiment of the invention, while the recipient is still undergoing continuous T cell depletion immunosuppressive therapy, the HSC are transplanted from the donor to the recipient patient. The recipient thymus has been activated by GnRH treatment and infiltrated by exogenous HSC.

In one embodiment hematopoietic stem cells (HSC) are given to the patient to speed the reactivation of the thymus. These may be autologous or syngeneic, but HSC from a mismatched donor (allogeneic or xenogeneic) can also be used. Where practical, the level of HSC in the patient's or donor's blood is enhanced by injecting the patient or donor with granulocyte-colony stimulating factor (G-CSF) at 10 μg/kg for 2-5 days prior to cell collection. CD34⁺ cells are purified from the patient's or a donor's blood or bone marrow, such as by using a flow cytometer or immunomagnetic beading. HSC are identified by flow cytometry as being CD34⁺. Optionally these HSC are expanded ex vivo with Stem Cell Factor. At approximately 1-3 weeks post LHRH agonist delivery, just before or at the time the thymus begins to regenerate, the patient is injected with the HSC, optimally at a dose of about 2-4×10⁶ cells/kg. Optionally G-CSF may also be injected into the recipient to assist in expansion of the HSC.

When the HSC are from a mismatched donor, T cell ablation and immunosuppressive therapy may be applied to the recipient to prevent rejection of the foreign HSC. In an example of such therapy, anti-T cell antibodies in the form of a daily injection of 15 mg/kg of Atgam (xeno anti-T cell globulin, Pharmacia Upjohn) are administered for a period of 10 days in combination with an inhibitor of T cell activation, cyclosporin A, 3 mg/kg, as a continuous infusion for 3-4 weeks followed by daily tablets at 9 mg/kg as needed. The prevention of T cell reactivity may also be combined with inhibitors of second level signals such as interleukins or cell adhesion molecules to enhance the T cell ablation. This treatment is begun before or at the same time as the beginning of sex steroid ablation.

Within about 3-4 weeks of LHRH therapy the first new T cells will be present in the blood stream of the recipient. However, in order to allow production of a stable chimera of host and donor hematopoietic cells, immunosuppressive therapy may be maintained for about 3-4 months. The new T cells will be purged of potentially donor reactive and host reactive cells, due to the presence of both donor and host DC in the reactivating thymus. Having been positively selected by the host thymic epithelium, the T cells will retain the ability to respond to normal infections by recognizing peptides presented by host APC in the peripheral blood of the recipient. The incorporation of donor dendritic cells into the recipient's lymphoid organs establishes an immune system situation virtually identical to that of the host alone, other than the tolerance of donor cells, tissue and organs. Hence, normal immunoregulatory mechanisms are present. These may also include the development of regulatory T cells which switch on or off immune responses using cytokines such as IL4, 5, 10, TGF-beta, TNF-alpha.

### Example 9

### Immunization and Prevention of Viral Infection (Influenza)

Influenza viruses are segmented RNA viruses that cause highly contagious acute respiratory infections. These viruses are endemic in man, where they are particularly devastating for the very young and the very old. The major problem associated with vaccine development against influenza is that these viruses have the ability to escape immune surveillance and remain in a host population. This escape is associated with changes in antigenic sites on the hemagglutinin (HA) and neuraminidase (N) envelope glycoproteins by phenomena termed antigenic drift and antigenic shift. Antigenic drift occurs when a subtype of an influenza virus H (for example H3) is selected for antigenic determinants that are not recognized by the anti-H3 antibody present in a population. This allows the virus to superinfect individuals who have already been infected by an H3 virus. Antigenic shift occurs when the influenza virus segmented genome reassorts to acquire an H belonging to another subtype (for example H2 instead of H3). The primary correlate for protection against influenza virus is neutralizing antibody against HA protein that undergoes strong selection for antigenic drift and shift. However, much more conserved antigenic cross-reactivities for different strains of influenza virus occur between internal proteins, such as the nucleoprotein (NP) (Shu, Bean and Webster, 1993). CTL and protection from influenza challenge following immunization with a polynucleotide encoding NP has previously been shown (Science 259:1745 (1993).

### Materials and Methods

Surgical Castration. BALB/c mice are anesthetized by intraperitoneal injection of 30-40 μl of a mixture of 5 ml of 100 mg/ml ketamine hydrochloride (Ketalar; Parke-Davis, Caringbah, NSW, Australia) plus 1 ml of 20 mg/ml xylazine (Rompun; Bayer Australia Ltd., Botany NSW, Australia) in saline. Surgical castration is performed as described elsewhere herein by a scrotal incision, revealing the testes, which are tied with suture and then removed along with surrounding fatty tissue. The wound is closed using surgical staples. Sham-castrated mice prepared following the above procedure without removal of the testes are used as controls.

Chemical castration. Mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen/ml) should normally be achieved by 3-4 weeks post injection.

Preparation of influenza A/PR/8/34 subunit vaccine. Purified influenza A/PR/8/34 (H1N1) subunit vaccine preparation is prepared following methods known in the art. Briefly, the surface hemagglutinin (HA) and neuraminidase (NA) antigens from influenza A/PR/8/34 particles are extracted using a non-ionic detergent (7.5% N-octyl-β-o-thioglucopyranoside). After centrifugation, the HA/NA-rich supernatant (55% HA) is used as the subunit vaccine.

Influenza A/PR/8/34 subunit immunization. Approximately 6 weeks following surgical castration or about 8 weeks following chemical castration, mice are immunized with 100 μl of formalin-inactivated influenza A/PR/8/34 virus (about 7000 HAU) injected subcutaneously. At these time points, thymic rejuvenation has occurred in both models of castration and the peripheral T cell pool has been replenished with naïve T cells recently exported from the thymus. The loss of sex steroids can also have a marked effect on the stimulatory capacity of new and pre-existing T cells in that they show a markedly enhanced proliferation to stimulation by antigen, which can occur within 7-10 days post surgical castration.

Booster immunizations can optionally be performed at about 4 weeks (or later) following the primary immunization. Freund's complete adjuvant (CFA) is used for the primary immunization and Freund's incomplete adjuvant is used for the optional booster immunizations.

Alternatively, the influenza A/PR/8/34 subunit vaccine preparation (see above) may be intramuscularly injected directly into, e.g., the quadriceps muscle, at a dose of about 1 μg to about 10 μg dilute in a volume of 40 μl sterile 0.9% saline.

Plasmid DNA. Preparation of plasmid DNA expression vectors are readily known in the art (see, e.g., Current Protocols In Immunology, Unit 2.14, John E. Coligan et al. (eds), Wiley and Sons, New York, N.Y. 1994, and yearly updates including 2002). Briefly, the complete influenza A/PR/8/34 nucleoprotein (NP) gene or hemagglutinin (HA) coding sequence is cloned into an expression vector, such as, pCMV, which is under the transcriptional control of the cytomegalovirus (CMV) immediate early promoter.

Empty plasmid (e.g., pCMV with no insert) is used as a negative control. Plasmids are grown in Escherichia coli DH5α or HB101 cells using standard techniques and purified using QIAGEN ULTRA-PURE-100 columns (Chatsworth, Calif.) according to manufacturer's instructions. All plasmids are verified by appropriate restriction enzyme digestion and agarose gel electrophoresis. Purity of DNA preparations is determined by optical density readings at 260 and 280 nm. All plasmids are resuspended in TE buffer and stored at −20° C. until use.

DNA immunization. Methods of DNA immunization are well known in the art. For instance, methods of intradermal, intramuscular, and particle-mediated (“gene gun” ) DNA immunizations are described in detail in, e.g., Current Protocols In Immunology, Unit 2.14, John E. Coligan et al. (eds), Wiley and Sons, New York, N.Y. 1994, and yearly updates including 2002).

Cytokine-encoding DNAs are optionally administered to shift the immune response to a desired Th1- or a Th2-type immune response. Th1-inducing genetic adjuvants include, e.g., IFN-γ and IL-12. Th2-inducing genetic adjuvants include, e.g., IL-4, IL-5, and IL-10. For review of the preparation and use of Th1- and Th2- inducing genetic adjuvants in the induction of immune response, see, e.g., Robinson, et al. (2000) Adv. Virus Res. 55:1-74.

Influenza A/PR/8/34 virus challenge. In an effort to determine if castrated mice are better protected from influenza virus challenge (with and without vaccination) as compared to their sham-castrated counterparts, metofane-anesthetized mice are challenged by intranasal inoculation of 50 μl of influenza A/PR/8/34 (H1N1) influenza virus containing allantoic fluid diluted 10⁻⁴ in PBS/2% BSA (50-100 LD₅₀; 0.25 HAU). Mice are weighed daily and sacrificed following>20% loss of pre-challenge weight. At this dose of challenge virus, 100% of naïve mice should succumb to influenza infection by 4-6 days.

Sublethal infections are optionally done prior assays to activate memory T cells, but use a 10⁻⁷ dilution of virus. Sublethal infections may also be optionally done to determine if non-immunized, castrated mice have better immune responses than the sham castrated controls, as determined by ELISA, cytokine assays (Th), CTL assays, etc. outlined below. Viral titers for lethal and sublethal infections may be optimized prior to use in these experiments.

Enzyme-linked immunosorbant assays. At various time periods pre- and post-immunization (or pre- and post- infection), mice from each group are bled, and individual mouse serum is tested using standard quatitative enzyme-linked immunosorbant assays (ELISA) to assess anti-HA or -NP specific IgG levels in the serum. IgG1 and IgG2a levels may optionally be tested, which are known to correlate with Th2 and Th1-type antibody responses, respectively. Briefly, sucrose gradient-purified A/PR/8/34 influenza virus is disrupted in flu lysis buffer (0.05 M Tris-HCL (pH 7.5-7.8), 0.5% TritonX-100, 0.6 M KCl) for 5 minutes at room temperature. Ninety-six well ELISA plates (Corning, Corning, N.Y.) are coated with 200 HAU influenza in carbonate buffer (0.8 g Na₂CO₃, 1.47 g NaHCO₃, 500 ml ddH₂O, pH to 9.6) and incubated overnight 4° C. Plates are blocked with 200 μl of 1% BSA in PBS for 1 hour at 37° C. and washed 5 times with PBS/0.025% Tween-20. Samples and standards are diluted in Standard Dilution Buffer (SDB) (0.5% BSA in PBS), added to microtiter plates at 50 μl per well, and incubated at 37° C. for 90 min. Following binding of antibody, plates are washed 5 times. Fifty microliters of HRP-labeled goat anti-mouse Ig subtype antibody (Southern Biotechnology Associates) is then added at optimized concentrations in SDB, and plates are incubated for 1 hour at 37° C. After washing plates 5 times, 100 μl of ABTS substrate (10 ml 0.05 M Citrate (pH 4.0), 5 ul 30% H₂O_{2b, 50} ul 40 mM ABTS) is added. Color is allowed to develop at room temperature for 30 min., and the reaction is stopped by adding 10 μl of 10% SDS. Plates are read at O.D.₄₀₅. Data are analyzed using Softmax Pro Version 2.21 computer software (Molecular Devices, Sunnyvale, Calif.).

Preparation and stimulation of splenocytes for cytokine production. Spleens are harvested from the various groups of mice (n=2-3) and pooled in p60 petri dishes containing about 4 ml RPMI-10 media (RPMI-1640, 10% fetal bovine serum, 50 μg/ml gentamycin). All steps in splenocyte preparations and stimulations are done aseptically. Spleens are minced with curved scissors into fine pieces and then drawn through a 5 cc syringe attached to an 18G needle several times to thoroughly resuspend cells. Cells are then expelled through a nylon mesh strainer into a 50 ml polypropylene tube. Cells are washed with RPMI-10, red blood cells were lysed with ACK lysis buffer (Sigma, St. Louis, Mo.), and washed 3 more times with RPMI-10. Cells were then counted by trypan blue exclusion, and resuspended in RPMI-10 containing 80 U/ml rat IL-2 (Sigma, St. Louis, Mo.) to a final cell concentration of 2×10⁷ cells/ml. Cells to be used for intracellular cytokine staining are stimulated in 96-well flat-bottom plates (Becton Dickenson Labware, Lincoln Park, N.J.), and cells to be used for cytokine analysis of bulk culture supernatants are stimulated in 96-well U-bottom plates (Becton Dickenson Labware, Lincoln Park, N.J.). One hundred microliters of cells are dispensed into wells of a 96-well tissue culture plate for a final concentration of 2×10⁶ cells/well. Stimulations are conducted by adding 100 1 of the appropriate peptide or inactivated influenza virus diluted in RPMI-10. CD8⁺ T cells are stimulated with either the K^{d}-restricted HA₅₃₃₋₅₄₁ peptide (IYSTVASSL) (Winter, Fields, and Brownlee, 1981) or the K^{d}-restricted NP₁₄₇₋₁₅₅ peptide (TYQRTRALV) Rotzchke et al., 1990). CD4⁺ T cells are stimulated with inactivated influenza virus (13,000 HAU per well of boiled influenza virus plus 13,000 HAU per well of formalin-inactivated influenza virus) plus anti-CD28 (1 μg/ml) and anti-CD49d (1 μg/ml) (Waldrop et al., 1998). Negative control stimulations are done with media alone. Cells are then incubated as described below to detect extracellular cytokines by ELISA or intracellular cytokines by FACS staining.

Chromium release assay for CTL. CTL responses to influenza HA and NP are measured using procedures well known to those in the art (see, e.g., Current Protocols In Immunology, John E. Coligan et al. (eds), Unit 3, Wiley and Sons, New York, N.Y. 1994, and yearly updates including 2002). The synthetic peptide HA₅₃₃₋₅₄₁ IYSTVASSL (Winter, Fields, and Brownlee, 1981) or NP₁₄₇₋₁₅₅ TYQRTRALV (Rotzschke et al., 1990) are used as the peptide in the target preparation step. Responder splenocytes from each animal are washed with RPMI-10 and resuspended to a final concentration of 6.3×10⁶ cells/ml in RPMI-10 containing 10 U/ml rat IL-2 (Sigma, St. Louis, Mo.). Stimulator splenocytes are prepared from naïve, syngeneic mice and suspended in RPMI-10 at a concentration of 1×10⁷ cells/ml. Mitomycin C is added to a final concentration of 25 μg/ml. Cells are incubated at 37° C./5% CO₂ for 30 minutes and then washed 3 times with RPMI-10. The stimulator cells are then resuspended to a concentration of 2.4×10⁶ cells/ml and pulsed with HA peptide at a final concentration of 9×10⁻⁶M or with NP peptide at a final concentration of 2×10⁻⁶M in RPMI-10 and 10 U/ml IL-2 for 2 hours at 37° C./5% CO₂. The peptide-pulsed stimulator cells (2.4×10⁶) and responder cells (6.3×10⁶) are then co-incubated in 24-well plates in a volume of 2 ml SM media (RPMI-10, 1 mM non-essential amino acids, 1 mM sodium pyruvate) for 5 days at 37° C./5% CO₂. A chromium-release assay is used to measure the ability of the in vitro stimulated responders (now called effectors) to lyse peptide-pulsed mouse mastocytoma P815 cells (MHC matched, H-2d). P815 cells are labeled with ⁵¹Cr by taking 0.1 ml aliquots of p815 in RPMI-10 and adding 25 μl FBS and 0.1 mCi radiolabeled sodium chromate (NEN, Boston, Mass.) in 0.2 ml normal saline. Target cells are incubated for 2 hours at 37° C./5%CO₂, washed 3 times with RPMI-10 and resuspended in 15 ml polypropylene tubes containing RPMI-10 plus HA (9×10⁻⁶M) or NP (1×10⁻⁶) peptide. Targets are incubated for 2 hours at 37° C./5%CO₂. The radiolabeled, peptide-pulsed targets are added to individual wells of a 96-well plate at 5×10⁴ cells per well in RPMI-10. Stimulated responder cells from individual immunization groups (now effector cells) are collected, washed 3 times with RPMI-10, and added to individual wells of the 96-well plate containing the target cells for a final volume of 0.2 ml/well. Effector to target ratios are 50:1, 25:1, 12.5:1 and 6.25:1. Cells are incubated for 5 hours at 37° C./5%CO₂ and cell lysis is measured by liquid scintillation counting of 25 μl aliquots of supernatants. Percent specific lysis of labeled target cells for a given effector cell sample is [100×(Cr release in sample-spontaneous release sample)/(maximum Cr release-spontaneous release sample)]. Spontaneous chromium release is the amount of radioactive released from targets without the addition of effector cells. Maximum chromium release is the amount of radioactivity released following lysis of target cells after the addition of TritonX-100 to a final concentration of 1%. Spontaneous release should not exceed 15%.

Detection of IFNγ or IL-5 in bulk culture supernatants by ELISA. Bulk culture supernatants may be tested for IFNγ and IL-5 cytokine levels, which are known to correlate with Th1 and Th2-type response, respectively. Pooled splenocytes are incubated for 2 days at 37° C. in a humidified atmosphere containing 5% CO₂. Supernatants are harvested, pooled and stored at −80° C. until assayed by ELISA. All ELISA antibodies and purified cytokines are purchased from Pharmingen (San Diego, Calif.). Fifty microliters of purified anti-cytokine monoclonal antibody diluted to 5 μg/ml (rat anti-mouse IFNγ) or 3 γg/ml (rat anti-mouse IL-5) in coating buffer (0.1M NaHCO₃, pH 8.2) is distributed per well of a 96-well ELISA plate (Corning, Corning, N.Y.) and incubated overnight at 4° C. Plates are washed 6 times with PBS/0.025% Tween-20 (PBS-T) and blocked with 250 μl of 2% dry milk/PBS for 90 min. at 37° C. Plates are washed 6 times with PBS-T. Standards (recombinant mouse cytokine) and samples are added to wells at various dilutions in RPMI-10 and incubated overnight at 4° C. for maximum sensitivity. Plates are washed 6 times with PBS-T. Biotinylated rat anti-mouse cytokine detecting antibody is diluted in PBS-T to a final concentration of 2 μg/ml and 100 μl was distributed per well. Plates are incubated for 1 hr. at 37° C. and then washed 6 times with PBS-T. Streptavidin-AP (Gibco BRL, Grand Island, N.Y.) is diluted 1:2000 according to manufacturer's instructions, and 100 μl is distributed per well. Plates are incubated for 30 min. and washed an additional 6 times with PBS-T. Plates are developed by adding 100 μl/well of AP developing solution (BioRad, Hercules, Calif.) and incubating at room temperature for 50 minutes. Reactions are stopped by addition of 100 μl 0.4M NaOH and read at OD₄₀₅. Data are analyzed using Softmax Pro Version 2.21 computer software (Molecular Devices, Sunnyvale, Calif.).

Intracellular cytokine staining and FACS analysis. Splenocytes may be tested for intracellular IFNγ and IL-5 cytokine levels, which are known to correlate with Th1 and Th2-type response, respectively. Pooled splenocytes are incubated for 5-6 hours at 37° C. in a humidified atmosphere containing 5% CO₂. A Golgi transport inhibitor, Monensin (Pharmingen, San Diego, Calif.), is added at 0.14 μl/well according to the manufacturer's instructions, and the cells are incubated for an additional 5-6 hours (Waldrop et al., 1998). Cells are thoroughly resuspended and transferred to a 96-well U-bottom plate. All reagents (GolgiStop kit and antibodies) are purchased from Pharmingen (San Diego, Calif.) unless otherwise noted, and all FACS staining steps are done on ice with ice-cold reagents. Plates are washed 2 times with FACS buffer (1×PBS, 2% BSA, 0.1% w/v sodium azide). Cells are surface stained with 50 μl of a solution of 1:100 dilutions of rat anti-mouse CD8β-APC, -CD69-PE, and -CD16/CD32 (FcγIII/RII; ‘Fc Block’) in FACS buffer. For tetramer staining (see below), cells were similarly stained with CD8β-TriColor, CD69-PE, CD16/CD32, and HA- or NP-tetramer-APC in FACS buffer. Cells are incubated in the dark for 30 min. and washed 3 times with FACS buffer. Cells are permeabilized by thoroughly resuspending in 100 μl of Cytofix/Cytoperm solution per well and incubating in the dark for 20 minutes. Cells are washed 3 times with Permwash solution. Intracellular staining is completed by incubating 50 μl per well of a 1:100 dilution of rat anti-mouse IFNγ-FITC in Permwash solution in the dark for 30 min. Cells are washed 2 times with Permwash solution and 1 time with FACS buffer. Cells are fixed in 200 μl of 1% paraformaldehyde solution and transferred to microtubes arranged in a 96-well format. Tubes are wrapped in foil and stored at 4° C. until analysis (less than 2 days). Samples are analyzed on a FACScan® flow cytometer (Becton Dickenson, San Jose, Calif.). Compensations are done using single-stained control cells stained with rat anti-mouse CD8-FITC, -PE, -TriColor, or -APC. Results are analyzed using FlowJo Version 2.7 software (Tree Star, San Carlos, Calif.).

Tetramers. HA and NP tetramers may be used to quantitate HA- and NP-specific CD8⁺ T cell responses following HA or NP immunization. Tetramers are prepared essentially as described previously (Flynn et al., 1998). The present example utilizes the H-2K^{d} MHC class I glycoprotein complexed the synthetic influenza A/PR/8/34 virus peptide HA₅₃₃₋₅₄₁ (IYSTVASSL) (Winter, Fields, and Brownlee, 1981) or NP₁₄₇₋₁₅₅ (TYQRTRALV) (Rotzschke et al., 1990).

It is noted that the methods described in this example are applicable to a wide array agents, with only minor variations, which would be readily determinable by those skilled in the art.

### Example 10

### Immunization and Prevention of Parasitic Infection (Malaria)

The circumsporozoite protein (CSP) is a target of this pre-erythocytic immunity (Hoffman et al. Science 252: 520 (1991). In the Plasmodium yoelii (P. yoelii) rodent model system, passive transfer P. yoelii CSP-specific monoclonal antibodies (Charoenvit et al., J. Immunol. 146: 1020 (1991)), as well as adoptive transfer of P. yoelii CSP-specific CD8⁺ T cells (Rodrigues et al., Int. Immunol. 3: 579 (1991), Weiss et al., J. Immunol. 149: 2103 (1992)) and CD4⁺ T cells (Renia et al. J Immunol. 150:1471 (1993)) are protective. Numerous vaccines designed to protect mice against sporozoites by inducing immune responses against the P. yoelii CSP have been evaluated.

Any Plasmodium sporozoite proteins known in the art capable of inducing protection against malaria usable in this invention may be used, such as P. falciparum, P. vivax, P. malariae, and P. ovale CSP; SSP2(TRAP); Pfs16 (Sheba); LSA-1; LSA-2; LSA-3; MSA-1 (PMMSA, PSA, p185, p190); MSA-2 (Gymmnsa, gp56, 38-45 kDa antigen); RESA (Pf155); EBA-175; AMA-1 (Pf83); SERA (p113, p126, SERP, Pf140); RAP-1; RAP-2; RhopH3; PfHRP-II; Pf55; Pf35; GBP (96-R); ABRA (p101); Exp-1 (CRA, Ag5.1); Aldolase; Duffy binding protein of P. vivax; Reticulocyte binding proteins; HSP70-1 (p75); Pfg25; Pfg28; Pfg48/45; and Pfg230.

### Materials and Methods

Surgical Castration. BALB/c mice are anesthetized by intraperitoneal injection of 30-40 μl of a mixture of 5 ml of 100 mg/ml ketamine hydrochloride (Ketalar; Parke-Davis, Caringbah, NSW, Australia) plus 1 ml of 20 mg/ml xylazine (Rompun; Bayer Australia Ltd., Botany NSW, Australia) in saline. Surgical castration is performed as described elsewhere herein by a scrotal incision, revealing the testes, which are tied with suture and then removed along with surrounding fatty tissue. The wound is closed using surgical staples. Sham-castrated mice prepared following the above procedure without removal of the testes are used as controls.

Chemical castration. Mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen/ml) should normally be achieved by 3-4 weeks post injection.

Parasites. The 17XNL (nonlethal) strain of P. yoelii is used as described previously (U.S. Pat. No. 5,814,617).

Preparation of irradiated P. yoelii sporozoites. Preparation of irradiated P. yoelii sporozoites for immunization has been described previously (see, e.g., Franke et al. Infect Immun. 68:3403 (2000)). Briefly, sporozoites are isolated by the discontinuous gradient technique (Pacheco et al., J. Parisitol. 65:414 (1979)) from infected Anopheles stephens mosquitoes that have been irradiated at 10,000 rads (¹³⁷Ce).

Immunization with irradiated P. yoelii sporozoites. Mice are intraveniously immunized with 50,000 sporozoites at approximately 6 weeks following surgical castration or about 8 weeks following chemical castration via the tail vein. Booster immunizations of 20,000 to 30,000 sporozoites are optionally given at 4 weeks and 6 weeks following the primary immunization (see, e.g., Franke et al. Infect Immun. 68:3403 (2000)).

Plasmid DNA and DNA immunization. Plasmid DNA encoding the full length P. yoelli CSP are known in the art. For instance, the pyCSP vector described in detail in Sedegah et al. (Proc. Natl. Acad. Sci. USA 95:7648 (1998)) may be used.

Methods of DNA immunization are also well known in the art. For instance, methods of intradermal, intramuscular, and particle-mediated (“gene gun” ) DNA immunizations are described in detail in, e.g., Current Protocols In Immunology, Unit 2.14, John E. Coligan et al. (eds), Wiley and Sons, New York, N.Y. 1994, and yearly updates including 2002).

Peptide Immunization. Methods of P. yoelii CSP peptide preparation are known in the art (see, e.g., Franke et al. Infect Immun. 68:3403 (2000)).

Chromium release assay for CTL. Since CD8⁺ CTL against the P. yoelii CSP have been shown to adoptively transfer protection (Weiss et al., J. Immunol. 149: 2103 (1992)), and CD8⁺ T cells are required for the protection against P. yoelii induced by immunization with irradiated sporozoites (Weiss et al., Proc. Natl. Acad. Sci USA 85: 573 (1988)), it must be determined if P. yoelii CSP vaccination (e.g., irradiated sporozoite, CSP peptide, or CSP DNA immunizations) elicits a CSP-specific CTL.

CTL responses are measured using procedures well known to those in the art (see, e.g., Current Protocols In Immunology, John E. Coligan et al. (eds), Unit 3, Wiley and Sons, New York, N.Y. 1994, and yearly updates including 2002). The general procedure described elsewhere herein for influenza HA and NP is used except that the cells are pulsed with the synthetic P. yoelli CSP peptide (281-296; SYVPSAEQILEFVKQI).

Inhibition of liver stage development assay. The liver stage development assay and acquisition of mouse hepatocytes from mouse livers by in situ collagenase perfusion have been described previously (Franke et al., Vaccine 17:1201 (1999); Franke et al., Infect Immun. 68:3403 (2000)). Hepatocyte cultures are seeded onto eight-chamber Lab-Tek plastic slides at 1×10⁵ cells/chamber and incubated with 7.5×10⁴ P. yoelli sporozoites for 3 hours. The cultures are then washed and cultured for and additional 24 hours at 37° C./5% CO₂. Effector cells are obtained as described above for the chromium release assay for CTL and are added and cultured with the infected hepatocytes for about 24-48 hours. The cultures are then washed, and the chamber slides are fixed for 10 min. in ice-cold absolute methanol. The chamber slides are then incubated with a monoclonal antibody (NYLS1 or NYLS3, both described previously in U.S. Pat. No. 5,814,617) directed against liver stage parasites of P. yoelii before incubating with FITC-labeled goat anti-mouse Ig. The number of liver-stage schizonts in triplicate cultures is then counted using an epifluorescence microscope. Percent inhibition is calculated using the formula [(control-test)/control)×100].

Infection and challenge. For a lethal challenge dose, the ID₅₀ of P. yoelli sporozoites must be determined prior to experimental challenge. However, for example, it is also initially possible to inject mice intravenously in the tail vein with a dose of about 50 to 100 P. yoelii sporozoites (nonlethel, strain 17XNL). Forty-two hours after intravenous inoculation, mice are sacrificed and livers are removed. Single cell suspensions of hepatocytes in medium are prepared, and 2×10⁵ hepatocytes are placed into each of 10 wells of a multi-chamber slide. Slides may be dried and frozen at −70° C. until analysis. To count the number of schizonts, slides are dried and incubated with NYLS 1 before incubating with FITC-labeled goat anti-mouse Ig, and the numbers of liver-stage schizonts in each chamber are counted using fluorescence microscopy.

Once it is demonstrated that castration and/or immunization reduces the numbers of infected hepatocytes, blood smears are obtained to determine if immunization protect against blood stage infection. Mice can be considered protected if no parasites are found in the blood smears at days 5-14 days post-challenge.

To test the preventative efficacy of castration alone (no vaccination) from a P. yoelli sporozoite primary infection, castrated mice are infected and analyzed as described above. Sham-castrated mice are used as controls.

Human studies. After establishing the efficacy in mice, large numbers of humans are immunized in a double blind placebo controlled field trial.

### Example 11

### Immunization and Prevention of Bacterial Infection (TB Ag85)

Tuberculosis (TB) is a chronic infectious disease of the lung caused by the pathogen Mycobacterium tuberculosis, and is one of the most clinically significant infections worldwide. (see, e.g., U.S. Pat. No. 5,736,524; for review see Bloom and Murray, 1993, Science 257, 1055

M. tuberculosis is an intracellular pathogen that infects macrophages. Immunity to TB involves several types of effector cells. Activation of macrophages by cytokines, such as IFNγ, is an effective means of minimizing intracellular mycobacterial multiplication. Acquisition of protection against TB requires both CD8⁺ and CD4⁺ T cells (see, e.g., Orme et al., J. Infect. Dis. 167, 1481 (1993)). These cells are known to secrete Th1-type cytokines, such as IFNγ, in response to infection, and possess antigen-specific cytotoxic activity. In fact, it is known in the art that CTL responses are useful for protection against M. tuberculosis (see, e.g., Flynn et al., Proc. Natl. Acad. Sci. USA 89, 12013 91992).

Predominant T cell antigens of TB are those proteins that are secreted by mycobacteria during their residence in macrophages. These T cell antigens include, but are not limited to, the antigen 85 complex of proteins (85A, 85B, 85C) (Wiker and Harboe, Microbiol. Rev. 56, 648(1992) and ESAT-6 (Andersen, Infect. Immunity, 62:2536 (1994)). Other T cell antigens have also been described in the art, see, e.g., Young and Garbe, Res. Microbiol. 142:55 (1991); Andersen, J. Infect. Dis. 166: 874 (1992); Siva and Lowrie, Immunol. 82:244 (1994); Romain et al, Proc. Natl. Acad. Sci. USA 90, 5322 (1993); and Faith et al, Immunol. 74:1 (1991).

The genes for each of the three antigen 85 proteins (A, B, and C) have been cloned and sequenced (see, e.g., Borremans et al., Infect. Immunity 57: 3123 (1989)); DeWit et al., DNA Seq. 4, 267 (1994)), and have been shown to elicit strong T cell responses following both infection and vaccination.

### Materials and Methods

Castration of mice. BALB/c or C57BL/6 mice are anesthetized by intraperitoneal injection of 30-40 μl of a mixture of 5 ml of 100 mg/ml ketamine hydrochloride (Ketalar; Parke-Davis, Caringbah, NSW, Australia) plus 1 ml of 20 mg/ml xylazine (Rompun; Bayer Australia Ltd., Botany NSW, Australia) in saline. Surgical castration is performed as described elsewhere herein by a scrotal incision, revealing the testes, which are tied with suture and then removed along with surrounding fatty tissue. The wound is closed using surgical staples. Sham-castrated mice prepared following the above procedure without removal of the testes are used as controls.

Chemical castration. Mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen/ml) should normally be achieved by 3-4 weeks post injection.

Protein immunization. General methods for Mycobacterium tuberculosis (TB) bacilli purification and immunization are known in the art (see, e.g., Current Protocols In Immunology, Unit 2.4, John E. Coligan et al. (eds), Wiley and Sons, New York, N.Y. 1994, and yearly updates including 2002). The purified TB may be prepared using preparative SDS-PAGE. Approximately 2 mg of the TB protein is loaded across the wells of a standard 1.5 mm slab gel using a large-tooth comb. An edge of the gel may be removed and stained following electrophoresis to identify the TB protein band on the gel. The gel region that contains the TB protein band is then sliced out of the gel, placed in PBS at a final concentration 0.5 mg purified TB protein per ml, and stored at 4° C. until use. The purified TB protein may then be emulsified with an equal volume of complete Freund's adjuvant (CFA) for immunization.

Approximately 6 weeks following surgical castration or about 8 weeks following chemical castration, 2 ml of the purified TB (0.5 mg/ml in PBS) is emulsified 2 ml CFA and stored at 4° C. The TB/CFA mixture is slowly drawn into and expelled through a 3-ml glass syringe attached to a 19 gauge needle, being certain to avoid excessive air bubbles. Once the emulsion is at a homogenous concentration, the needle is replaced by a 22 gauge needle, and all air bubbles are removed. The castrated and sham-castrated mice are injected intramuscularly with a 50 μl volume of the TB/CFA emulsion (immunization may also be done via the intradermal or subcutaneous routes). M. bovis BCG may also be used in a vaccine preparation.

A booster immunization can optionally be performed 4-8 weeks (or later) following the primary immunization. The TB adjuvant emulsion is prepared in the same manner described above, except that incomplete Freund's adjuvant (IFA) is used in place of CFA for all booster immunizations. Further booster immunizations can be performed at 2-4 week (or later intervals) thereafter.

Plasmid DNA. Suitable Ag85-encoding DNA sequences and vectors have been described previously. See, e.g., U.S. Pat. No. 5,736,524. Other suitable expression vectors would be readily ascertainably by hose skilled in the art.

Antigen 85 DNA Immunization. Methods of DNA immunization are well known in the art. For instance, methods of intradermal, intramuscular, and particle-mediated (“gene gun” ) DNA immunizations are described in detail in, e.g., Current Protocols In Immunology, Unit 2.14, John E. Coligan et al. (eds), Wiley and Sons, New York, N.Y. 1994, and yearly updates including 2002).

Cytokine-encoding DNAs are optionally administered to shift the immune response to a desired Th1- or a Th2-type immune response. Th1-inducing genetic adjuvants include, e.g., IFN-γ and IL-12. Th2-inducing genetic adjuvants include, e.g., IL-4, IL-5, and IL-10. For review of the preparation and use of Th1- and Th2- inducing genetic adjuvants in the induction of immune response, see, e.g., Robinson, et al. (2000) Adv. Virus Res. 55:1-74.

Approximately 6 weeks following surgical castration or about 8 weeks following chemical castration, mice are intramuscularly injected with 200 μg of DNA diluted in 100 μl saline.

Booster DNA immunizations are optionally administered at 4 weeks post-prime and 2 weeks post-boost.

Enzyme-linked immunosorbant assays. At various time periods pre- and post-immunization, mice from each group are bled, and individual mouse serum is tested using standard quantitative ELISA to assess anti-Ag85 specific IgG levels in the serum. IgG1 and IgG2a levels may optionally be tested, which are known to correlate with Th2 and Th-type antibody responses, respectively.

Serum is collected at various time points pre- and post-prime and post boost, and analyzed for the presence of anti-Ag85 specific antibodies in serum. Basic ELISA methods are described elsewhere herein, except purified Ag85 protein is used.

Cytokine assays. Spleen cells from vaccinated mice are analyzed for cytokine secretion in response to specific Ag85 restimulation, as described, e.g., in Huygen et al, Infect. Immunity 60:2880 (1992) and U.S. Pat. No. 5,736,524. Briefly, spleen cells are incubated with culture filtrate (CF) proteins from M. bovis BCG purified Ag85A or the C57BL/6 T cell epitope peptide (amino acids 241-260).

Four weeks post-prime and 2 weeks post boost (or later), cytokines are assayed using standard bio-assays for IL-2,IFNγ and IL-6, and by ELISA for IL-4 and IL-10 using methods well known to those in the art. See, e.g., Current Protocols In Immunology, Unit 6, John E. Coligan et al. (eds), Wiley and Sons, New York, N.Y. 1994, and yearly updates including 2002.

Mycobacterial infection and challenge. To test the efficacy of the vaccinations, mice are challenged by intravenous injection of live M. bovis BCG (0.5 mg). At various time points post-challenge, BCG multiplication is analyzed in both mouse spleens and lungs. Positive controls are naïve mice (castrated and/or sham castrated as appropriate) receiving a challenge dose.

To test the efficacy of sex steroid ablation to prevent primary infection, live M. bovis BCG are injected similarly to that described in the challenge experiment above. Sham castrated mice are used as controls.

The number of colony-forming units (CFU) in the spleen and lungs of the challenged, vaccinated mice, as well as in the lungs of the castrated, primary infected mice is expected to be substantially lower than in negative control animals, which is indicative with protection in the live M. bovis challenge model.

### Example 12

### Immunization and Prevention of Cancer

To determine if sex steroid ablation is effective in preventing cancer and/or in eliciting a protective immune response following vaccination with a cancer antigen, the following studies are performed.

### Materials and Methods

Castration of mice. C57BL/6 mice are anesthetized by intraperitoneal injection of 30-40 μl of a mixture of 5 ml of 100 mg/ml ketamine hydrochloride (Ketalar; Parke-Davis, Caringbah, NSW, Australia) plus 1 ml of 20 mg/ml xylazine (Rompun; Bayer Australia Ltd., Botany NSW, Australia) in saline. Surgical castration is performed as described elsewhere herein by a scrotal incision, revealing the testes, which are tied with suture and then removed along with surrounding fatty tissue. The wound is closed using surgical staples. Sham-castrated mice prepared following the above procedure without removal of the testes are used as controls.

Chemical castration. Mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen /ml) should normally be achieved by 3-4 weeks post injection.

CEA immunization. Approximately 6 weeks following surgical castration or about 8 weeks following chemical castration, mice were inoculated with an adenovirus vector encoding the human carcinoembryonic antigen (CEA) gene (MC38-CEA-2) (Conry et al., 1995), such as AdCMV-hcea described in U.S. Pat. No. 6,348,450. Alternatively, a plasmid DNA encoding the human CEA gene is injected into the mouse (e.g., intramuscularly into the quadriceps muscle) utilizing one of the various methods of DNA vaccination described elsewhere herein.

Tumor challenge. To assess the efficacy of sex steroid ablation on anti-tumor activity of mice immunized with CEA, mice are subjected to a tumor challenge. At various time points post immunization, syngeneic tumor cells expressing the human CEA gene (MC38-CEA-2) (Conry et al., 1995) are inoculated into the mice. Mice are observed every other day for development of palpable tumor nodules. Mice are sacrificed when the tumor nodules exceed 1 cm in diameter. The time between inoculation and sacrifice is the survival time.

To test the efficacy of sex steroid ablation preventing tumors, tumor cells expressing the human CEA gene are inoculated into castrated, non-vaccinated mice as outlined above. Sham castrated mice are used as controls.

### Example 13

### Transplantation of Genetically Modified HSC (Gene Therapy)

I. SCID-hu Mouse Model

### Materials and Methods

Mice. SCID-hu mice are prepared essentially as described previously (see, e.g. Namikawa et al., J. Exp. Med. 172:1055 (1990) and Bonyhadi et al., J. Virol. 71:4707 (1997) by surgical transplantation of human fetal liver and thymus fragments into CB-17 scid/scid mice. Methods for the construction of SCID-hu Thy/Liv mice can also be found, e.g., in Current Protocols In Immunology, Unit 4.8, John E. Coligan et al. (eds), Wiley and Sons, New York, N.Y. 1994, and yearly updates including 2002.

Castration of mice. The SCID-hu mice are anesthetized by intraperitoneal injection of 30-40 μl of a mixture of 5 ml of 100 mg/ml ketamine hydrochloride (Ketalar; Parke-Davis, Caringbah, NSW, Australia) plus 1 ml of 20 mg/ml xylazine_(Rompun; Bayer Australia Ltd., Botany NSW, Australia) in saline. Surgical castration is performed as described above by a scrotal incision, revealing the testes, which are tied with suture and then removed along with surrounding fatty tissue. The wound is closed using surgical staples. Sham-castrated mice prepared following the above procedure without removal of the testes are used as controls.

Chemical castration. Mice are injected subcutaneously with 10 mg/kg Lupron (a GnRH agonist) as a 1 month slow release formulation. Alternatively mice are injected with a GnRH antagonist (e.g., Cetrorelix or Abarelix). Confirmation of loss of sex steroids is performed by standard radioimmunoassay of plasma samples following manufacturer's instructions. Castrate levels (<0.5 ng testosterone or estrogen/ml) should normally be achieved by 3-4 weeks post injection.

Isolation of human CD34⁺ HSC. Human cord blood (CB) HSC are collected and processed using techniques well known to those skilled in the art (see, e.g., DiGusto et al., Blood, 87:1261 (1997), Bonyhadi et al., J. Virol. 71:4707 (1997)). A portion of each CB sample is HLA phonotyped for the MA2.1 surface molecule. CD34+ cells are enriched using immunomagnetic beads using the method described in Bonyhadi et al., J. Virol. 71:4707 (1997)). Briefly, CB cells are resuspended at a concentration of 5×10⁷ cells/ml RPMI containing 2% heat-inactivated fetal calf serum (FCS), 10 mM HEPES, and 1 mg/ml human gamma globulin, and incubated for 4° C. for 5 min. Four μg/ml of anti-CD34 antibody (QBEND-10, Immunotech) is added and the cells are incubated for 14 min. at 4° C. The cells are then washed and resuspended at a final concentration of 2×10⁷ cells/ml. CD34⁺ cells are then enriched using goat-anti-mouse IgG1 magnetic beads (Dynal) following manufacturer's instructions. The CD34⁺ cells are then incubated with 50 μl of glycoprotease (O-sialoglycoprotein endopeptidase), which causes release of the CD34⁺ cells from the immunomagnetic beads. The beads are removed using a magnet, and the cells are then subjected to flow cytometry using anti-CD34-PE and various other cell surface markers conjugated to either FITC or TRICOLOR to determine the total level of CD34⁺ cells present in the population.

Optionally, HSC are expanded ex vivo with IL-3, IL-6, and either SCF or LWF (10 ng/ml each).

RevM10 vectors and preparation of genetically modified (GM) HSC. RevM10 is known in the art, and has been described extensively in studies of GM HSC for the survival of T cells in HIV-infected patients (see, e.g., Woffendin et al., Proc. Natl. Acad. Sci. USA, 93:2889 (1996); for review, see Amado et al., Front. Biosci. 4:d468 (1999)). The HIV Rev protein is known to affect viral latency in HIV infected cells and is essential for HIV replication. RevM10 is a derivative of Rev because of mutations within the leucine-rich domain of Rev that interacts with cell factors. RevM10 has a substitution of aspartic acid for leucine at position 78 and of Leucine for glutamic acid at position 79. The result of these mutations is that RevM10 is able to compete effectively with the wild-type HIV Rev for binding to the Rev-responsive element (RRE).

Any of the RevM10 gene transfer vectors known and described in the art may be used. For example, the retroviral RevM10 vector, pLJ-RevM10 is used to transducer the HSC. The pLJ-RevM10 vector has been shown to enhance T cell engraftment after delivery into HIV-infected individuals (Ranga et al., Proc. Natl. Acad. Sci. USA 95:1201 (1998). Other methods of construction and retroviral vectors suitable for the preparation of GM HSC are well known in the art (see, e.g., Bonyhadi et al., J. Virol. 71:4707 (1997)).

In another example, the pRSV/TAR RevM10 plasmid is used for non-viral vector delivery using particle-mediated gene transfer into the isolated target HSC essentially as described in Woffendin et al., Proc. Natl. Acad. Sci. USA, 91:11581 (1994). The pRSV/TAR RevM10 plasmid contains the Rous sarcoma virus (RSV) promoter and tat-activation response element (TAR) from −18 to +72 of HIV is used to express the RevM10 open reading frame may also be used (Woffendin et al., Proc. Natl. Acad. Sci. USA, 91:11581 (1994); Liu et al., Gene Ther. 1:32 (1997)). In vitro transfection of this plasmid into human PBL has previously been shown to provide resistance to HIV infection (Woffendin et al., Proc. Natl. Acad. Sci. USA, 91:11581 (1994)).

A marker gene, such as the Lyt-2α (murine CD8α) gene, may also be incorporated into the RevM10 vector for ease of purification and analysis of GM HSC by FACS analysis in subsequent steps (see, e g., Bonyhadi et al., J. Virol. 71:4707 (1997)).

A ΔRev10, which contains a deletion of the methionine (Met) initiation codon (ATG), as well as a linker comprising a series of stop codons inserted in-frame into the BglII site of the RevM10 gene, is constructed and used as a negative control (see, e g., Bonyhadi et al., J. Virol. 71:4707 (1997)).

Injection of GM HSC into mice. SCID-hu mice are analyzed, and the mice determined to be HLA mismatched (MA2.1) with respect to the human donor HSC are given approximately 400 rads of total body irradiation (TBI) about four months following the thymic and liver grafts in an effort to eliminate the cell population. After TBI, mice are reconstituted with the RevM10 GM HSC (see above) as described previously (see, e.g., DiGusto et al., Blood, 87:1261 (1997), Bonyhadi et al., J. Virol. 71:4707 (1997)). Control mice are injected with unmodified HSC or with HSC that have been modified with the ΔRevM10 gene or an irrelevant gene.

Analysis of GM HSC by flow cytometry. Approximately 8 to 12 weeks after GM HSC reconstitution, the Thy/Liv grafts are removed, and the thymocytes are obtained and analyzed for the HLA pheonotype (MA2.1) and the distribution of CD4⁺, CD8⁺, and Lyt2 (the “marker” murine homolog of CD8α) surface expression using methods of flow cytometry and FACS analysis readily known to those skilled in the art (see, e.g., Bonyhadi et al., J. Virol. 71:4707 (1997)); see also Current Protocols In Immunology, Units 4.8 and 5, John E. Coligan et al. (eds), Wiley and Sons, New York, N.Y. 1994, and yearly updates including 2002). Thymocytes are also tested for transgenic DNA with primers specific for the RevM10 gene using standard PCR methods.

Analysis of GM HSC resistance to HIV infection. Approximately 8 to 12 weeks (or later) after GM HSC reconstitution, the Thy/Liv grafts are removed and the thymocytes are obtained from the GM HSC reconstituted SCID-hu mice. The thymocytes are stimulated in vitro and infected with the JR-CSF molecular isolate of HIV-1 as described previously (Bonyhadi et al., J. Virol. 71:4707 (1997)). Briefly, the thymocytes are stimulated in vitro in the presence of irradiated allogeneic feeder cells (106 peripheral blood mononuclear cells/ml and 10⁵ JY cells/ml) in RPMI medium containing 10% FCS, 50 μg/mi streptomycin, 50 U/G penicillin G, 1×MEM vitamin solution, 1×insulin transferring-sodium selenite medium supplement (Sigma), 40 U human rIL-2/ml, and 2 μg/ml phytohemagglutinin (PHA) (Sigma). About every 10 days, cells are restimulated with feeder cells and PHA as described previously in Vandekerckhove et al., J. Exp. Med. 1:1033 (1992). Approximately 5 days after stimulation, cells were sorted on the basis of donor HLA phenotype (MA2. 1) and Lyt2 (the “marker” murine homolog of CD8α). Sorted cells are restimulated and may be expanded to increase the cell composition to greater than about 90% purity. CD4⁺/Lyt2⁺ cells are then sorted out and an aliquot of approximately 5×10⁴ of the sorted cells are place in multiple wells of a 96-well U bottom tissue culture plate. About 200 TCID₅₀ of EW, an HIV-1 primary isolate, or 1000 TCID₅₀ of JR-CSF, an HIV-1 molecular isolate, are added to each well. Methods of virus stock preparation have been described previously (Bonyhadi et al. Nature, 363:728 (1993). Medium is changed every day from days 3 to 12. Aliquots of supernatant are collected every other day and stored at −80° C. until use. Tissue culture supernatants are then analyzed using a p24 ELISA following manufacturer's instructions (Coulter).

II. Therapy of HIV Infected Individual

### Materials and Methods

Isolation of human CD34⁺ HSC. As most HIV infected patients have very low titers of HSC, it is possible to use a donor to supply cells. Where practical, the level of HSC in the donor blood is enhanced by injecting into the donor granulocyte-colony stimulating factor (G-CSF) at 10 μg/kg for 2-5 days prior to cell collection.

In this example, human cord blood (CB) HSC are collected and processed using techniques well known to those skilled in the art (see, e.g., DiGusto et al., Blood, 87:1261 (1997), Bonyhadi et al., J. Virol. 71:4707 (1997)). A portion of each CB sample is HLA phonotyped, and the CD34⁺ donor cells are purified from the donor blood (or bone marrow), such as by using a flow cytometer or immunomagnetic beading, essentially as described above. Donor-derived HSC are identified by flow cytometry as being CD34⁺.

Optionally, HSC are expanded ex vivo with IL-3, IL-6, and either SCF or LWF (10 ng/ml each).

RevM10 vectors and preparation of genetically modified (GM) HSC. Any of the RevM10 gene transfer vectors known and described in the art, including those described in the mouse studies above, may be used. Methods of gene transduction using GM retroviral vectors or gene transfection using particle-mediated delivery are also well known in the art, and are described elsewhere herein.

As described above, a retroviral vector may be constructed to contain the trans-dominant mutant form of HIV-1 rev gene, RevM10, which has been shown to inhibit HIV replication (Bonyhadi et al. 1997). Amphotropic vector-containing supernatants are generated by infection with filtered supernatants from ecotropic producer cells that were transfected with the vector.

The collected CD34⁺ cells are optionally pre-stimulated for 24 hours in LCTM media supplemented with IL-3, IL-6 and SCF or LWF (10 ng/ml each) to induce entry of the cells into the cell cycle.

In this example, CD34⁺-enriched HSC undergo transfection by a linearized RevM10 plasmid utilizing particle-mediated (“gene gun” transfer) essentially as described in Woffendin et al., Proc. Natl. Acad. Sci. USA, 93:2889 (1996).

However, if retroviral transduction is done, supernatants containing the vectors are repeatedly added to the cells for 2-3 days to allow transduction of the vectors into the cells.

HAART Treatment of HIV-infected patients. HAART therapy is begun before T cell depletion and sex steroid ablation, and therapy is maintained throughout the procedure to reduce the viral titer.

T cell depletion. T cell depletion is performed to remove as many HIV infected cells as possible. It is also performed to remove T cells recognizing non-self antigens to allow for use of nonautologous, genetically modified cells. One standard procedure for this step is as follows. The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment does not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus. The prevention of T cell reactivity may also be combined with inhibitors of second level signals such as interleukins or cell adhesion molecules to enhance the T cell ablation.

This depletion of peripheral T cells minimizes the risk of graft rejection because it depletes non-specifically all T cells including those potentially reactive against a foreign donor. Simultaneously, however, because of the lack of T cells the procedure induces a state of generalized immunodeficiency which means that the patient is highly susceptible to infection, particularly viral infection. Even B cell responses will not function normally in the absence of appropriate T cell help.

Sex steroid ablation therapy. The HIV-infected patient is given sex steroid ablation therapy in the form of delivery of an LHRH agonist. This is given in the form of either Leucrin (depot injection; 22.5 mg) or Zoladex (implant; 10.8 mg), either one as a single dose effective for 3 months. This is effective in reducing sex steroid levels sufficiently to reactivate the thymus. In some cases it is also necessary to deliver a suppresser of adrenal gland production of sex steroids. Cosudex (5 mg/day) as one tablet per day may be delivered for the duration of the sex steroid ablation therapy. Adrenal gland production of sex steroids makes up around 10-15% of a human's steroids. Alternatively, the patient is given a GnRH antagonist, e.g., Cetrorelix or Abarelix, as a subcutaneous injection.

Reduction of sex steroids in the blood to minimal values takes about 1-3 weeks post surgical castration, and about 3-4 weeks following chemical castration. Concordant with this is the reactivation of the thymus. In some cases it is necessary to extend the treatment to a second 3 month injection/implant.

In the event of a shortened time available for transplantation of donor genetically modified cells, the timeline is modified: T cell ablation and sex steroid ablation may be begun at the same time. T cell ablation is maintained for about 10 days, while sex steroid ablation is maintained for around 3 months.

Injection of GM HSC into patients. Prior to injection, the GM HSC are expanded in culture for approximately 10 days in X-Vivo 15 medium comprising I1-2 (Chiron, 300 IU/ml).

At approximately 1-3 weeks post LHRH agonist delivery, just before or at the time the thymus begins to reactivate, the patient is injected with the genetically modified HSC, optimally at a dose of about 2-4×10⁶ cells/kg. Optionally G-CSF may also be injected into the recipient to assist in expansion of the GM HSC.

Immediately prior to patient infusion, the GM HSC are washed four times with Dulbecco's PBS. Cells are resuspended in 100 ml of saline comprising 1.25% human albumin and 4500 U/ml IL-2, and infused into the patient over a course of 30 minutes.

Following sex steroid ablation, thymus reactivation, and injection of the GM HSC in the HIV-infected patient, all new T cells (as well as DC, macrophages, etc.) will be resistant to subsequent infection by this virus. Injection of allogeneic HSC into a patient undergoing thymic reactivation means that the HSC will enter the thymus. The reactivated thymus takes up the genetically modified HSC and converts them into donor-type T cells and dendritic cells, while converting the recipient's HSC into recipient-type T cells and dendritic cells. By inducing deletion by cell death, or by inducing tolerance through immunoregulatory cells, the donor dendritic cells will tolerize any T cells that are potentially reactive with recipient.

When the thymic chimera is established, and the new cohort of mature T cells have begun exiting the thymus, reduction and eventual elimination of immunosuppression occurs.

Post-infusion studies. Following infusion, the persistence and half life of GM HSC in the HIV-infected patient is be tested periodically using limiting dilution PCR of PBL samples obtained from the patient essentially as described in Woffendin et al., Proc. Natl. Acad. Sci. USA, 93:2889 (1996). The relative level of GM HSC in the infected patient is compared to the negative control patient that received the ΔRevM10 vector.

Various standard hematologic (e.g., CD4+ T cell counts), immunologic (e.g., neutralizing antibody titers), and virologic (e.g., viral titer) studies will also be performed using methods well known to those skilled in the art.

Termination of immunosuppression. When the thymic chimera is established and the new cohort of mature T cells have begun exiting the thymus, blood is taken from the patient and the T cells examined in vitro for their lack of responsiveness to donor cells in a standard mixed lymphocyte reaction (see, e.g., Current Protocols In Immunology, Unit 3.12, John E. Coligan et al. (eds), Wiley and Sons, New York, N.Y. 1994, and yearly updates including 2002). If there is no response, the immunosuppressive therapy is gradually reduced to allow defense against infection. If there is no sign of rejection, as indicated in part by the presence of activated T cells in the blood, the immunosuppressive therapy is eventually stopped completely. Because the HSC have a strong self-renewal capacity, the hematopoietic chimera so formed will be stable theoretically for the life of the patient (as for normal, non-tolerized and non-grafted people).

### Example 14

### Alternative Protocols

In the event of a shortened time available for transplantation of donor cells, tissue or organs, the timeline as used in Examples 1-14 is modified. T cell ablation and sex steroid ablation may be begun at the same time. T cell ablation is maintained for about 10 days, while sex steroid ablation is maintained for around 3 months. In one embodiment, HSC transplantation is performed when the thymus starts to reactivate, at around 10-12 days after start of the combined treatment.

In an even more shortened time table, the two types of ablation and the HSC transplant may be started at the same time. In this event T cell ablation may be maintained 3-12 months, and, in one embodiment, for 3-4 months.

When the thymic chimera is established and the new cohort of mature T cells have begun exiting the thymus, blood is taken from the patient and the T cells examined in vitro for their lack of responsiveness to donor cells in a standard mixed lymphocyte reaction (see, e.g., Current Protocols In Immunology,John E. Coligan et al. (eds), Wiley and Sons, New York, N.Y. 1994, and yearly updates including 2002). If there is no response, the immunosuppressive therapy is gradually reduced to allow defense against infection. If there is no sign of rejection, as indicated in part by the presence of activated T cells in the blood, the immunosuppressive therapy is eventually stopped completely. Because the HSC have a strong self-renewal capacity, the hematopoietic chimera so formed will be stable theoretically for the life of the patient (as for normal, non-tolerized and non-grafted people).

### Example 15

### Regeneration of the Thymus

The reactivated thymus takes up the HSC and converts them into new T cells, which emigrate into the blood stream and rebuild an optimal peripheral T cell pool in the patient. In particular, there is a major increase in the levels and proportions of naïve T cells, which greatly increases the number of potential responding cells in vaccination programs. The correct ratios of Th:Tc and Th1:Th2 cells ensures an optimal type of response.

When the new cohort of mature T cells have begun exiting the thymus, blood is taken from the patient and the status of T cells (and indeed all blood cells) is examined. In particular the T cells are examined for whether they are Th1 or Th2, and naïve or memory. In addition the types of cytokines they produce (Th1 versus Th2) are examined.

Immunosuppressive therapy, if used due to administration of mismatched HSC, is gradually reduced to allow defense against infection, and is stopped completely when there is no sign of rejection, as indicated in part by the presence of activated T cells in the blood. Because the HSC have a strong self-renewal capacity, the hematopoietic chimera formed will be stable, theoretically for the life of the patient, as in the situation where no mismatched HSC are used.

### Example 16

### Termination of Immunosuppression

When the thymic chimera is established and the new cohort of mature T cells have begun exiting the thymus, blood is taken from the patient and the T cells examined in vitro for their lack of responsiveness to donor cells in a standard mixed lymphocyte reaction (see, e.g., Current Protocols In Immunology, John E. Coligan et al. (eds), Wiley and Sons, New York, N.Y. 1994, and yearly updates including 2002). If there is no response, the immunosuppressive therapy is gradually reduced to allow defense against infection. If there is no sign of rejection, as indicated in part by the presence of activated T cells in the blood, the immunosuppressive therapy is eventually stopped completely. Because the HSC have a strong self-renewal capacity, the hematopoietic chimera so formed will be stable theoretically for the life of the patient (as for normal, non-tolerized and non-grafted people).

### Example 17

### Use of LHRH Agonist to Reactive the Thymus in Humans

### Materials and Methods

In order to show that a human thymus can be reactivated by the methods of this invention, these methods were used on patients who had been treated with chemotherapy for prostate cancer.

Patients. Sixteen patients with Stage I-III prostate cancer (assessed by their prostate specific antigen (PSA) score) were chosen for analysis. All subjects were males aged between 60 and 77 who underwent standard combined androgen blockade (CAB) based on monthly injections of GnRH agonist 3.6 mg Goserelin (Zoladex) or 7.5 mg Leuprolide (Lupron) treatment per month for 4-6 months prior to localized radiation therapy for prostate cancer as necessary.

FACS analysis. The appropriate antibody cocktail (20 μl) was added to 200 μl whole blood and incubated in the dark at room temperature (RT) for 30 min. For removal of RBC, 2 ml of FACS lysis buffer (Becton-Dickinson, USA) was then added to each tube, vortexed and incubated 10 min., RT in the dark. Samples were centrifuged at 600_{gmax}; supernatant removed and cells washed twice in PBS/FCS/Az. Finally, cells were resuspended in 1% PFA for FACS analysis. Samples were stained with antibodies to CD19-FITC, CD4-FITC, CD8-APC, CD27-FITC, CD45RA-PE, CD45RO-CyChrome, CD62L-FITC and CD56-PE (all from Pharmingen, USA).

Statistical analysis. Each patient acted as an internal control by comparing pre- and post-treatment results and were analysed using paired student t-tests or Wilcoxon signed rank tests.

Results: Prostate cancer patients were evaluated before and 4 months after sex steroid ablation therapy. The results are summarized in FIGS. 30-34. Collectively the data demonstrate qualitative and quantitative improvement of the status of T cells in many patients.

### Results

I. The Effect of LHRH Therapy on Total Numbers of Lymphocytes and T Cells Subsets Thereof:

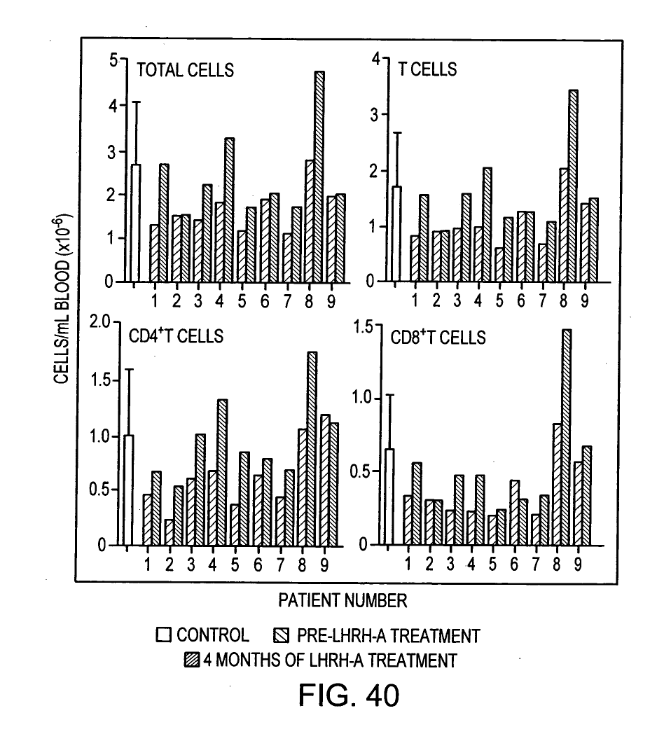

The phenotypic composition of peripheral blood lymphocytes was analyzed in patients (all>60 years) undergoing LHRH agonist treatment for prostate cancer (FIG. 40). Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4⁺ T cells. A less distinctive trend was seen within the CD8⁺ subset with 4/9 patients showing increased levels albeit generally to a smaller extent than CD4⁺ T cells.

II. The Effect of LHRH Therapy on the Proportion of T Cells Subsets:

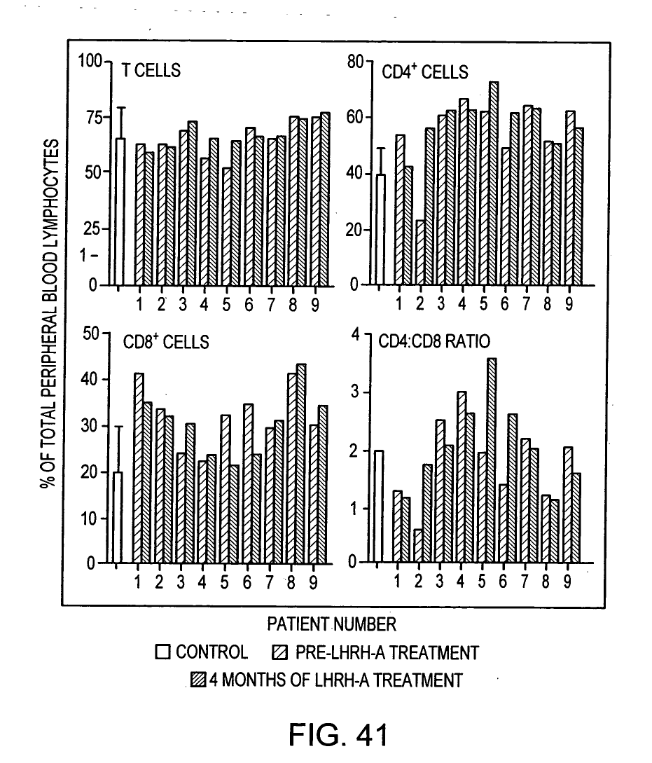

Analysis of patient blood before and after LHRH agonist treatment demonstrated no substantial changes in the overall proportion of T cells, CD4⁺ or CD8⁺ T cells and a variable change in the CD4⁺:CD8⁺ ratio following treatment (FIG. 41). This indicates that there was little effect of treatment on the homeostatic maintenance of T cell subsets despite the substantial increase in overall T cell numbers following treatment. All values were comparative to control values.

III. The Effect of LHRH Therapy on the Proportion of B Cells and Myeloid Cells:

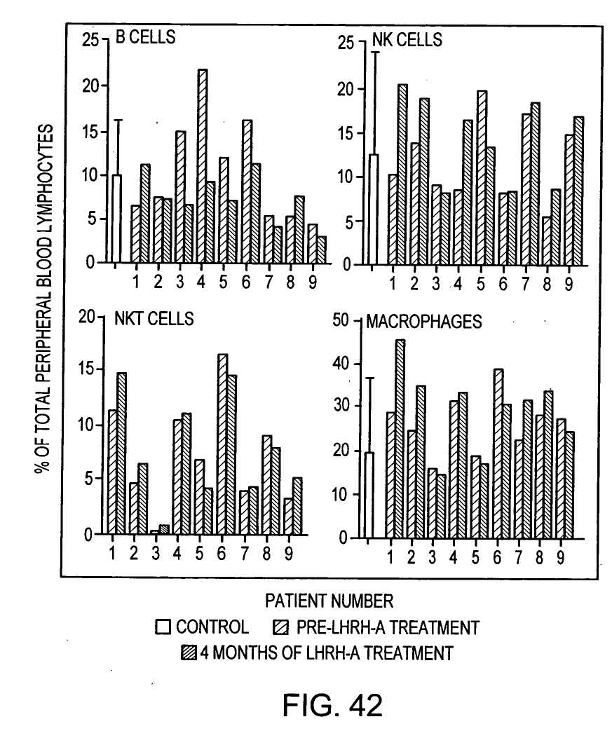

Analysis of the proportions of B cells and myeloid cells (NK, NKT and macrophages) within the peripheral blood of patients undergoing LHRH agonist treatment demonstrated a varying degree of change within subsets (FIG. 42). While NK, NKT and macrophage proportions remained relatively constant following treatment, the proportion of B cells was decreased in 4/9 patients.

IV. The Effect of LHRH Agonist Therapy on the Total Number of B Cells and Myeloid Cells:

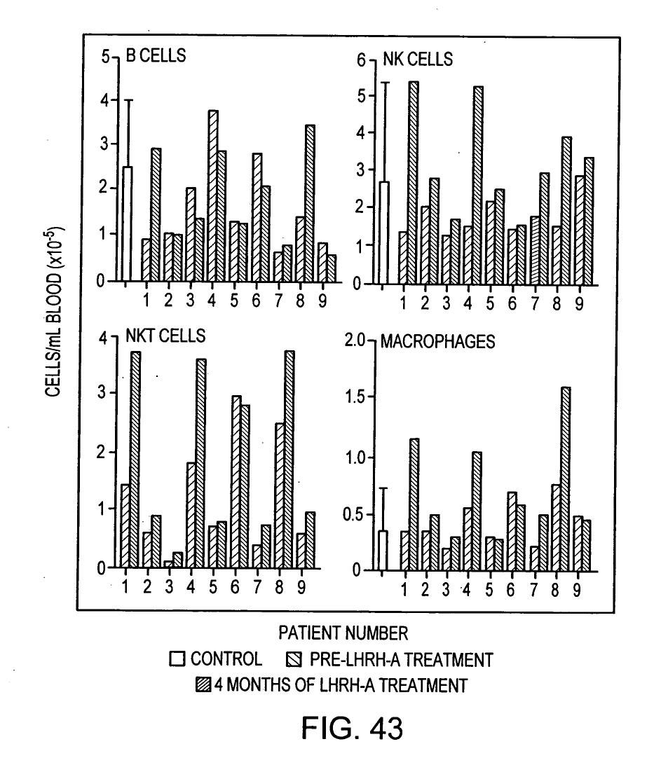

Analysis of the total cell numbers of B and myeloid cells within the peripheral blood post-treatment showed clearly increased levels of NK (5/9 patients), NKT (4/9 patients) and macrophage (3/9 patients) cell numbers post-treatment (FIG. 43). B cell numbers showed no distinct trend with 2/9 patients showing increased levels; 4/9 patients showing no change and 3/9 patients showing decreased levels.

V. The Effect of LHRH Therapy on the Level of Naïve Cells Relative to Memory Cells:

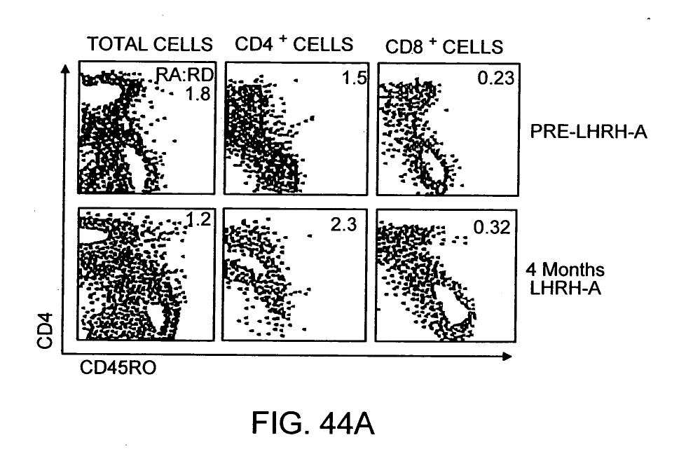

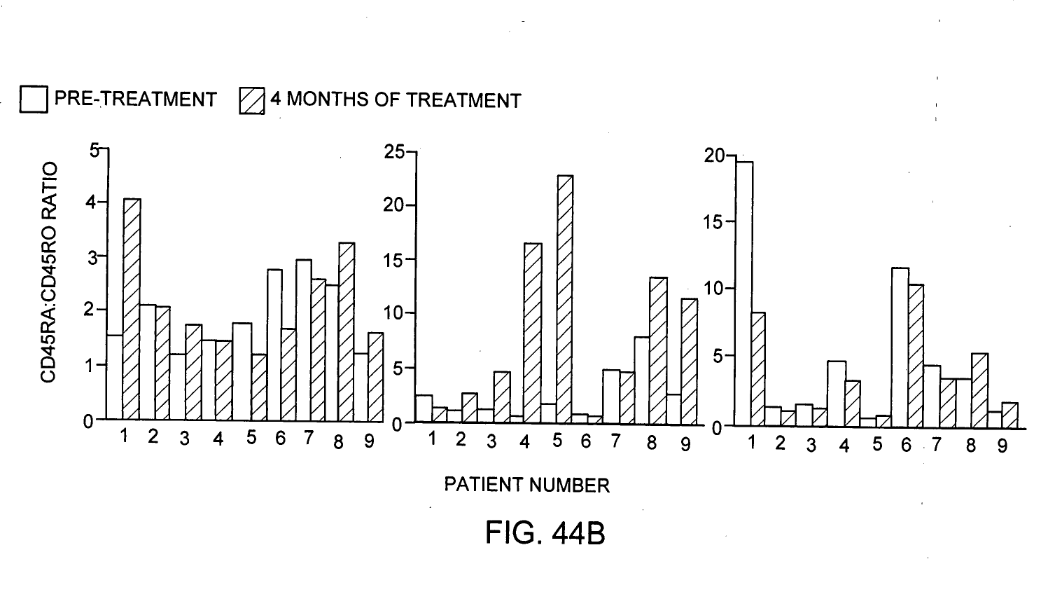

The major changes seen post-LHRH agonist treatment were within the T cell population of the peripheral blood. In particular there was a selective increase in the proportion of naïve (CD45RA⁺) CD4⁺ cells, with the ratio of naïve (CD45RA⁺) to memory (CD45RO⁺) in the CD4+T cell subset increasing in 6/9 patients (FIG. 44).

VI. Conclusion

Thus it can be concluded that LHRH agonist treatment of an animal such as a human having an atrophied thymus can induce regeneration of the thymus. A general improvement has been shown in the status of blood T lymphocytes in these prostate cancer patients who have received sex-steroid ablation therapy. While it is very difficult to precisely determine whether such cells are only derived from the thymus, this would be very much the logical conclusion as no other source of mainstream (TCRαβ+CD8 αβ chain) T cells has been described. Gastrointestinal tract T cells are predominantly TCR γδ or CD8 αα chain.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in biology or related fields are intended to be within the scope of the following claims.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### REFERENCES

Andrew, D. and Aspinall, R., 2001, “IL-7 and not stem cell factor reverses both the increase in apoptosis and the decline in thymopoiesis seen in aged mice.” J Immunol 166(3): 1524-1530.

Aspinall, R., 1997, “Age-associated thymic atrophy in the mouse is due to a deficiency affecting rearrangement of the TCR during intrathymic T cell development,” J. Immunol. 158:3037.

Atlan-Gepner, C., Naspetti, M., et al., 1999, “Disorganisation of thymic medulla precedes evolution towards diabetes in female NOD mice.” Autoimmunity 3:249-260.

Bahnson, A. B., et al., 1997, “Method for Retrovirus-Mediated Gene Transfer to CD34⁺-Enriched Cells,” in GENE THERAPY PROTOCOLS (P. D. Robbins, ed.), Humana Press, pp.249-263.

Bauer, G., et al., 1997, “Inhibition of Human Immunodeficiency Virus-1 (HIV-1) Replication After Transduction of Granulocyte Colony-Stimulating Factor-Mobilized CD34+ Cells From HIV-1- Infected Donors Using Retroviral Vectors Containing Anti-HIV-1 Genes,” Blood 89:2259-2267.

Belmont, J. W. and R. Jurecic, 1997, “Methods for Efficient Retrovirus-Mediated Gene Transfer to Mouse Hematopoietic Stem Cells,” in GENE THERAPY PROTOCOLS (P. D. Robbins, ed.), Humana Press, pp.223-240.

Berzins, S. P., Boyd, R. L. and Miller, J. F. A. P., 1998, “The role of the thymus and recent thymic migrants in the maintenance of the adult peripheral lymphocyte pool,” J Exp. Med. 187:1839.

Bonyhadi, M. L., et al., 1997, “RevM10-Expressing T Cells Derived In Vivo From Transduced Human Hematopoietic Stem-Progenitor Cells Inhibit Human Immunodeficiency Virus Replication,” J. Virology 71:4707-4716.

Boyd, R. L., Tucek, C. L., Godfrey, D. I., Wilson, T. J, Davidson, N. J., Bean, A. G. D., Ladyman, H. M., Ritter, M. A. and Hugo, P., 1993, “The thymic microenvironment,” Immunology Today 14:445.

Bruijntjes, J. P., Kuper, C. J., Robinson, J. E. and Schutirman, H. J., 1993, “Epithelium-free area in the thymic cortex of rats,” Dev. Immunol. 3:113.

Capecchi, M. R., 1980, “High Efficiency Transformation by Direct Microinjection of DNA Into Cultured Mammalian Cells,” Cell 22:479-488.

Carayon, P., and Bord, A., 1992, “Identification of DNA-replicating lymphocyte subsets using a new method to label the bromo-deoxyuridine incorporated into the DNA,” J. Imm. Methods 147:225.

Castle, S. C., 2000, “Clinical relevance of age-related immune dysfunction” Clin Infect Dis 31(2): 578-585.

Doria, G., Mancini, C., Utsuyama, M., Frasca, D. and Hirokawa, K, 1997, “Aging of the recipients but not of the bone marrow donors enhances autoimmunity in syngeneic radiation chimeras” Mech. Age. Dev. 95: 131-142.

Douek, D. C., McFarland, R. D., Keiser, P. H., Gage, E. A., Massey, J. M., Haynes, B. F., Polis, M. A., Haase, A. T., Feinberg, M. B., Sullivan, J. L., Jamieson, B. D., Zack, J. A., Picker, L. J. and Koup, R. A., 1998, “Changes in thymic function with age and during the treatment of HIV infection,” Nature 396:690.

Fredrickson, G. G. and Basch, R. S., 1994, “Early thymic regeneration after irradiation,” Development and Comparative Immunology 18:251.

George, A. J. and Ritter, M. A., 1996, “Thymic involution with ageing: obsolescence or good housekeeping?,” Immunol. Today 17:267.

Godfrey, D. I, Izon, D. J., Tucek, C. L., Wilson, T. J. and Boyd, R. L., 1990, “Thephenotypic heterogeneity of mouse thymic stromal cells,” Immunol. 70:66.

Godfrey, D. I, and Zlotnik, A., 1993, “Control points in early T-cell development,” Immunol. Today 14:547.

Graham, F. L. and Van Der Eb, A. J., 1973, “A New Technique for the Assay of Infectivity of Human Adenovirus 5 DNA,” Virology 52:456-457.

Haynes, B. F., Hale, L. P., Weinhold, K. J., Patel, D. D., Liao, H. -X., Bressler, P. B., Jones, D. M., Demarest, J. F., Gebhard-Mitchell, K., Haase, A. T. and Bartlett, J. A., 1999, “Analysis of the adult thymus in reconstitution of T lymphocytes in HIV-1 infection” J. Clin. Invest. 103: 453-460.

Heitger, A., Neu, N., Kern, H. and Fink, F. M., 1997, “Essential role of the thymus to reconstitute naive (CD45RA⁺) T helper cells after human allogeneic bone marrow transplantation,” Blood 90: 850-857.

Heitger, A., Winklehner, P., et al., 2002, “Defective T-helper cell function after T-cell-depleting therapy affecting naive and memory populations” Blood 99:4053-4062.

Hirokawa, K., 1998, “Immunity and Ageing,” in PRINCIPLES AND PRACNCE OF GERIATRIC MEDICINE, (M. Pathy, ed.) John Wiley and Sons Ltd.

Hirokawa, K. and Makinodan, T., 1975, “Thymic involution: the effect on T cell differentiation,” J. Immunol. 114:1659.

Hirokawa, K., Utsuyama M., Kasai, M., Kurashima, C., Ishijima, S. and Zeng, Y. -X., 1994, “Understanding the mechanism of the age-change of thymic function to promote T cell differentiation,” Immunology Letters 40:269.

Hobbs, M. V., Weigle, W. O., Noonan, D. J., Torbett, B. E., McEvilly, R. J., Koch, R. J., Cardenas, G. J. and Ernst, D. N., 1993, “Patterns of cytokine gene expression by CD4+ T cells from young and old mice,” J. Immunol. 150:3602.

Homo-Delarche, R. and Dardenne, M., 1991, “The neuroendocrine-immune axis,” Seminars in Immunopathology.

Huiskamp, R., Davids, J. A. G. and Vos, O., 1983, “Short- and long- term effects of whole body irradiation with fission neutrons or x-rays on the thymus in CBA mice,” Radiation Research 95:370.

Kamradt, T. and Mitchison, N. A., 2001, Advances in immunology: Tolerance and autoimmunity.” N.Engl. J. Med. 344:655-664.

Kendall, M. D., 1988, “Anatomical and physiological factors influencing the thymic microenvironment,” in THYMUS UPDATE I, Vol. 1. (M. D. Kendall, and M. A. Ritter, eds.) Harwood Academic Publishers, p. 27.

Kohn, D. B., et al., 1999, “A Clinical Trial of Retroviral-Mediated Transfer of a rev-Responsive Element Decoy Gene Into CD34⁺Cells From the Bone Marrow of Human Immunodeficiency Virus-1 Infected Children,” Blood 94:368-371.

Kurashima, C, Utsuyama, M., Kasai, M., Ishijima, S. A., Konno, A. and Hirokawa, A., 1995, “The role of thymus in the aging of Th cell subpopulations and age-associated alteration of cytokine production by these cells,” Int. Immunol. 7:97.

Mackall, C. L. et. al., 1995, “Age, thymopoiesis and CD4+ T-lymphocyte regeneration after intensive chemotherapy,” New England J. Med. 332:143.

Mackall, C. L. and Gress, R. E., 1997, “Thymic aging and T-cell regeneration,” Immunol. Rev. 160:91.

Mackall, C. L., Punt, J. A., Morgan, P., Farr, A. G. and Gress, R. E., 1998, “Thymic function in young/old chimeras: substantial thymic T cell regenerative capacity despite irreversible age-associated thymic involution” Eur. J. Immunol. 28: 1886-1893.

Murasko, D. M., Bernstein, E. D., et al., 2002, “Role of humoral and cell-mediated immunity in protection from influenza disease after immunization of healthy elderly.” Exp. Gerontol. 37:427-439.

Nabel, E. G., et al., 1992, “Gene Transfer In Vivo With DNA-Liposome Complexes: Lack of Autoimmunity and Gonadal Localization,” Hum. Gene Ther. 3:649-656.

Panoskaltsis, N, and C. N. Abboud, 1999, “Human Immunodeficiency Virus and the Hematopoietic Repertoire: Implications For Gene Therapy,” Frontiers in Bioscience 4:457.

Penit, C. and Ezine, S., 1989, “Cell proliferation and thymocyte subset reconstitution in sublethally irradiated mice: compared kinetics of endogenous and intrathymically transferred progenitors,” Proc. Natl. Acad. Sci, U.S.A. 86:5547.

Penit, C., Lucas, B., Vasseur, F., Rieker, T. and Boyd, R. L., 1996, “Thymic medulla epithelial cells acquire specific markers by post-mitotic maturation,” Dev. Immunol. 5:25.

Plosker, G. L. and Brogden, R. N., 1994, “Leuprorelin. A review of its pharmacology and therapeutic use in prostatic cancer, endometriosis and other sex hormone-related disorders,” Drugs 48:930.

Potter, H., Weir, L., and Leder, P., 1984, “Enhancer-dependent expression of Human Kappa Immunoglobulin Genes Introduced Into Mouse pre-B Lymphocytes by Electroporation,” Proc. Natl. Acad. Sci. USA 81:7161-7165.

Randle-Barrett, E. S. and Boyd, R. L., 1994, “Thymic microenvironment and lymphoid responses to sublethal irradiation,” Dev. Immunol. 4:1.

Rossi, S., Blazar, B. R., et al., 2002, “Keratinocyte growth factor preserves normal thymopoiesis and thymic microenvironment during experimental graft-versus-host disease” Blood 100:682-691.

Scollay, R. G., Butcher, E. C. and Weissman, I. L., 1980, “Thymus cell migration. Quantitative aspects of cellular traffic from the thymus to the periphery in mice,” Eur. J. Immunol. 10:210.

Sempowski, G. D., Hale, L. P., Sundy, J. S., Massey, J. M., Koup, R. A., Douek, D. C., Patel, D. D. and Haynes, B. F., 2000, “Leukemia Inhibitory Factor, Oncostatin M, IL-6, and Stem Cell Factor mRNA expression in human thymus increases with age and is associated with thymic atrophy.” J. Immunology 164: 2180-2187.

Shortman, K., Egerton, M., Spangrude, G. J. and Scollay, R., 1990, “The generation and fate of thymocytes,” Seminars in Immuno. 2:3.

Starzl, T. E., Demetris, A. J., Murase, N., Ricardi, C. and Truce, M., 1992, “Cell migration, chimerism, and graft acceptance,” Lancet 339:1579.

Strayer, D. S., Branco, F. et al., 2002, “Combination genetic therapy to inhibit HIV-1.” Molecular Therapy 5:33-41.

Suda, T., and Zlotnik, A., 1991, “IL-7 maintains the T cell precursor potential of CD3-CD4-CD8- thymocytes,” J. Immunol. 146:3068.

Takeoka, Y., Taguchi, N. et al., 1999, “Thymic microenvironment and NZB mice: The abnormal thymic microenvironment of New Zealand mice correlates with immunopathology.” Clin. Immunol. 90:388-398.

Timm, J. A. and Thoman, M. L., 1999, “Maturation of CD4+ lymphocytes in the aged microenviroment results in a memory-enriched population,” J. Immunol. 162:711.

Thomas-Vaslin, V., Damotte, D. et al., 1997, “Abnormal T cell selection on nod thymic epithelium is sufficient to induce autoimmune manifestations in C57BU6 athymic nude mice.” P.N.A.S. (U.S.A.) 94:4598-4603.

Thomson, A. W. and Lu, L., 1999, “Are dendritic cells the key to liver transplant?,” Immunology Today 20:20.

Tosi, R., Kraft, R., Luzi, P., Cintorino, M., Fankhause, G., Hess, M. W. and Cottier, H., 1982, “Involution pattern of the human thymus. 1. Size of the cortical area as a function of age,” Clin. Exp. Immunol. 47:497.

van Ewijk, W., Rouse, R. V. and Weissman, I. L., 1980, “Distribution of H-2 microenvironments in the mouse thymus,” J. Histochem. Cytochem. 28:1089.

Vickery, B. H., et al., eds., 1984, LHRH AND ITS ANALOGS: CONTRACEPTIVE & THERAPEUTC APPLICATIONS, MTP Press Ltd., Lancaster, Pa.

von Freeden-Jeffry, U., Vieira, P., Lucian, L. A., McNeil, T., Burdach, E. G. and Murray, R., 1995, “Lymphopenia in interleukin (IL)-7 gene-deleted mice identifies IL-7 as a nonredundant cytokine,” J. Exp. Med. 181:1519.

Weyand, C. M., Brandes, J. C., Schmidt, D., Fulbright, J. W. and Goronzy, J. J., 1998, “Functional properties of CD4⁺CD28⁻ T cells in the aging immune system” Mech. Age. Dev. 102: 131-147.

Wiles, M. V., Ruiz, P. and Imhof, B. A., 1992, “Interleukin-7 expression during mouse thymus development,” Eur. J. Immunol. 22:1037.

Yang, N. -S. and P. Ziegelhoffer, 1994, “The Particle Bombardment System for Mammalian Gene Transfer,” In PARTICLE BOMBARDMENT TECHNOLOGY FOR GENE TRANSFER (Yang, N. -S. and Christou, P., eds.), Oxford University Press, New York, pp. 117-141.

Zlotnik, A. and Moore, T. A., 1995,“Cytokine production and requirements during T-cell development,” Curr. Opin. Immunol. 7:206.

1-14 (Cancelled)

15. A method for improving an immune response to a vaccine antigen in a patient, comprising:
reactivating the thymus of the patient; and
administering a vaccine to the patient, the vaccine comprising a vaccine antigen,
wherein the patient develops an immune response to the vaccine antigen.

16. The method of claim 15, wherein the thymus of the patient has been at least in part atrophied before it is reactivated.

17. The method of claim 16, wherein the patient has a disease that at least in part atrophied the thymus of the patient.

18. The method of claim 16, wherein the patient has had a treatment of a disease that at least in part atrophied the thymus of the patient.

19. The method of claim 18, wherein the treatment is immunosuppression, chemotherapy, or radiation treatment.

20. The method of claim 16, wherein the patient is post-pubertal.

21. The method of claim 15, further comprising administering cells to the patient, wherein the cells are stem cells, progenitor cells, or combinations thereof.

22. The method of claim 21, wherein the stem cells are selected from the group consisting of hematopoietic stem cells, epithelial stem cells, and combinations thereof.

23. The method of claim 21, wherein the progenitor cells are selected from the group consisting of lymphoid progenitor cells, myeloid progenitor cells, and combinations thereof.

24. (Cancelled)

25. The method of claim 22, wherein the cells are hematopoietic stem cells.

26. The method of claim 25, wherein the hematopoietic stem cells are CD34⁺.

27. The method of claim 21, wherein the cells are autologous.

28. The method of claim 21, wherein the cells are not autologous.

29. The method of claim 25, wherein the hematopoietic stem cells are administered when the thymus begins to reactivate.

30. The method of claim 15, wherein the thymus is reactivated by disruption of sex steroid-mediated signaling to the thymus.

31. The method of claim 30, further comprising administering cells to the patient, wherein the cells are stem cells, progenitor cells, or combinations thereof.

32. The method of claim 31, wherein the stem cells are selected from the group consisting of hematopoietic stem cells, epithelial stem cells, and combinations thereof.

33. The method of claim 31, wherein the progenitor cells are selected from the group consisting of lymphoid progenitor cells, myeloid progenitor cells, and combinations thereof.

34. (Cancelled)

35. The method of claim 32, wherein the cells are hematopoietic stem cells.

36. The method of claim 31, wherein the cells are administered at the time disruption of sex steroid-mediated signaling to the thymus is begun.

37. The method of claim 30, wherein the sex steroid-mediated signaling to the thymus is disrupted by surgical castration.

38. The method of claim 30, wherein the sex steroid-mediated signaling to the thymus is disrupted by chemical castration.

39. The method of claim 30, wherein the sex steroid-mediated signaling to the thymus is disrupted by administration of a pharmaceutical.

40. The method of claim 39, wherein the pharmaceutical is selected from the group consisting of LHRH agonists, LHRH antagonists, anti-LHRH vaccines, anti-androgens, anti-estrogens, SERMs, SARMs, SPRMs, ERDs, aromatase inhibitors, anti-progestogens, Dioxalan derivatives and combinations thereof.

41. The method of, claim 40, wherein the LHRH agonists are selected from the group selected from the group consisting of Goserelin, Lupron, Leuprolide, Triptorelin, Meterelin, Buserelin, Histrelin, Nafarelin, Lutrelin, Leuprorelin, Deslorelin, Cystorelin, Decapeptyl, Gonadorelin, and combinations thereof.

42. The method of claim 40, wherein the LHRH antagonists are selected from the group consisting of Abarelix, Cetrorelix, and combinations thereof.

43. The method of claim 15, wherein patient's immune response to the vaccine antigen is improved compared to that immune response which would have otherwise occurred in a patient prior to thymus reactivation.

44. The method of claim 15, wherein the vaccine is a therapeutic vaccine or a prophylactic vaccine.

45. The method of claim 15, wherein the vaccine antigen is an antigen from an agent, wherein the agent is selected from the group consisting of a virus, a bacterium, a fungus, a parasite, a prion, a cancer, an allergen, an asthma-inducing agent, a “self” protein and an antigen which causes an autoimmune disease.

46. The method of claim 45, wherein the agent is a virus.

47. The method of claim 46, wherein the virus is selected from the group consisting of Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxviridae, and Iridoviridae.

48. The method of claim 46, wherein the virus is selected from the group consisting of influenza virus, human immunodeficiency virus, and herpes simplex virus.

49. The method of claim 45, wherein the agent is a bacterium.

50. The method of claim 41, wherein the bacterium is selected from the group consisting of Helicobacter pylori, Borelia burgdorferi, Legionella pneumophilia, Mycobacterium tuberculosis, Mycobacterium avium, Mycobacterium intracellulare, Mycobacterium kansaii, Mycobacterium gordonae, Mycobacteria sporozoites, Staphylococcus aureus, Neisseria gonorrhoeae, Neisseria meningitidis, Listeria monocytogenes, Streptococcus pyogenes, Streptococcus agalactiae, Streptococcus faecalis, Streptococcus bovis, Streptococcus pneumoniae, pathogenic Campylobacter sporozoites, Enterococcus sporozoites, Haemophilus influenzae, Bacillus anthracis, Corynebacterium diphtheriae, Corynebacterium sporozoites, Erysipelothrix rhusiopathiae, Clostridium perfringens, Clostridium tetani, Enterobacter aerogenes, Klebsiella pneumoniae, Pasturella multocida, Bacteroides sporozoites, Fusobacterium nucleatum, Streptobacillus moniliformis, Treponema pallidium, Treponema pertenue, Leptospira, and Actinomyces israelli.

51. The method of claim 49, wherein the bacterium is a mycobacterium.

52. The method of claim 45, wherein the agent is a parasite.

53. The method of claim 52, wherein the parasite is selected from the group consisting of Plasmodium falciparum, Plasmodium yoelli, and Toxoplasma gondii.

54. The method of claim 52, wherein the parasite is a malaria parasite.

55. The method of claim 45, wherein the agent is an infectious fungus.

56. The method of claim 55, wherein the infectious fungus is selected from the group consisting of Cryptococcus neoformans, Histoplasma capsulatum, Coccidioides immitis, Blastomyces dermatitidis, Chlamydia trachomatis, Candida albicans.

57. The method of claim 45, wherein the agent is a cancer or tumor.

58. The method of claim 57, wherein the cancer is selected from the group consisting of a cancer of the brain, a cancer of the lung, a cancer of the ovary, a cancer of the breast, a cancer of the prostate, a cancer of the colon, a cancer of the blood, a carcinoma, a melanoma and a sarcoma.

59. The method of claim 45, wherein the agent is an allergen.

60. The method of claim 59, wherein the allergen causes an allergic condition selected from the group consisting of eczema, allergic rhinitis, allergic coryza, hay fever, bronchial asthma, urticaria (hives), and food allergies.

61-62. (Cancelled)

63. The method of claim 15, wherein the vaccine is selected from the group consisting of killed vaccines, inactivated vaccines, attenuated vaccines, recombinant vaccines, subunit vaccines, and DNA vaccines.

64. The method of claim 15, wherein the vaccine is administered when the thymus begins to reactivate.

65. The method of claim 30, wherein the vaccine is administered at the time disruption of sex steroid-mediated signaling to the thymus is begun.

66. The method of claim 15, further comprising administering a cytokine, a growth factor, or a combination of a cytokine and a growth factor to the patient.

67. The method of claim 66, wherein the cytokine is selected from the group consisting of Interleukin 2 (IL-2), Interleukin 3 (IL-3), Interleukin 4 (IL-4), Interleukin 6 (IL-6), Interleukin 7 (IL-7), Interleukin 15 (IL-15), Interferon gamma (IFN-γ), and combinations thereof.

68. The method of claim 66, wherein the growth factor is selected from the group consisting of members of the epithelial growth factor family, members of the fibroblast growth factor family, stem cell factor, granulocyte colony stimulating factor (G-CSF), keratinocyte growth factor (KGF), insulin-like growth factor-1 (IGF-1), and combinations thereof.

69-71. (Cancelled)

72. A method for enhancing transplantation of donor hematopoietic stem cells into the thymus of a recipient patient, comprising:
depleting the T cells of the patient;
reactivating the thymus of the patient; and
transplanting donor hematopoietic stem cells to the patient,
wherein uptake of the donor hematopoietic stem cells into the patient's thymus is enhanced as compared to the uptake that would have otherwise occurred in a patient prior to thymus reactivation.

73. A method for increasing virus-specific peripheral T cell responsiveness of a patient with an at least partially atrophied thymus, comprising:
reactivating the thymus of the patient;
exposing the patient to a virus; and
determining the virus-specific peripheral T cell responsiveness in the patient,
wherein the patient has an increased viral-specific peripheral T cell responsiveness as compared to the responsiveness that would have otherwise occurred in a patient prior to thymus reactivation.

74. The method of claim 30, wherein the sex steroid-mediated signaling to the thymus is disrupted by lowering the level of sex steroid hormones.

75. The method of claim 15, wherein the method further comprises administering an adjuvant to the patient.

76. The method of claim 40, wherein the anti-androgen is Eulexin or ketoconazole.
